(19) United States
(12) Patent Application Publication
(10) Pub. No.: US 2009/0023747 A1
(43) Pub. Date: Jan. 22, 2009

(54) Cysteine Protease Inhibitors

(76) Inventors: David Tickle, Essex (GB); Kevin Parkes, Essex (GB); Matt Tozer, Essex (GB); Urszula Grabowska, Essex (GB)

Correspondence Address: BIRCH STEWART KOLASCH & BIRCH, PO BOX 747, FALLS CHURCH, VA, 22040-0747, US

(21) Appl. No.: 11/994,496

(22) PCT Filed: Jul. 6, 2006
(86) PCT No.: PCT/EP2006/063952

§ 371 Date: Mar. 19, 2008

(30) Foreign Application Priority Data
Jul. 7, 2005 (GB) 0513839.1

(51) Int. Cl. A61K 31/496; C07D 487/02; A61K 31/407; C07D 213/02; A61P 19/02; A61P 35/00; A61P 19/08; A61K 31/4439; C07D 295/00

(52) U.S. Cl. 514/254.02; 548/453; 514/412; 546/276.7; 514/338; 544/369

(57) ABSTRACT

A compound of the formula (II) wherein one of R¹ and R² is halo and the other is H or halo; R³ is —C₁-C₅ straight or branched chain, optionally fluorinated, alkyl or —CH₂CR⁵C₃-C₄-Cycloalkyl; R⁴ is H; R⁵ is H, C₁-C₂ alkyl, C₁-C₂ haloalkyl, hydroxyl, OC₁-C₂alkyl, fluoro; R⁶ is a stable, optionally substituted, monocyclic or bicyclic, carbocycle or heterocycle wherein the or each ring has 4, 5 or 6 ring atoms and 0 to 3 hetero atoms selected from S, O and N; Rb is haloalkyl; Rc is H or C₁-C₄ alkyl; and pharmaceutically acceptable salts, hydrates or N-oxides thereof have utility in the treatment of disorders characterised by inappropriate expression or activation of cathepsin K, such as osteoporosis, osteoarthritis, rheumatoid arthritis or bone metastases.

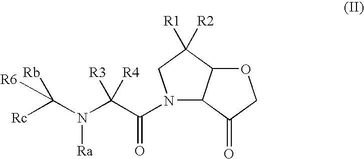

### FIELD OF THE INVENTION

This invention relates to inhibitors of cysteine proteases, especially those of the papain superfamily. The invention provides novel compounds useful in the prophylaxis or treatment of disorders stemming from misbalance of physiological proteases such as cathepsin K.

### DESCRIPTION OF THE RELATED ART

The papain superfamily of cysteine proteases is widely distributed in diverse species including mammals, invertebrates, protozoa, plants and bacteria. A number of mammalian cathepsin enzymes, including cathepsins B, F, H, K, L, O and S, have been ascribed to this superfamily, and inappropriate regulation of their activity has been implicated in a number of metabolic disorders including arthritis, muscular dystrophy, inflammation, glomerulonephritis and tumour invasion. Pathogenic cathepsin like enzymes include the bacterial gingipains, the malarial falcipains I, II, III et seq and cysteine proteases from Pneumocystis carinii, Trypanosoma cruzei and brucei, Crithidia fusiculata, Schistosoma spp.

The inappropriate regulation of cathepsin K has been implicated in a number of disorders including osteoporosis, gingival diseases such as gingivitis and periodontitis, Paget's disease, hypercalcaemia of malignancy and metabolic bone disease. In view of its elevated levels in chondroclasts of osteoarthritic synovium, cathepsin K is implicated in diseases characterised by excessive cartilege or matrix degradation, such as osteoarthritis and rheumatoid arthritis. Metastatic neoplastic cells typically express high levels of proteolytic enzymes that degrade the surrounding matrix and inhibition of cathepsin K may thus assist in treating neoplasias.

International patent application no WO02057270 discloses compounds of the formula I:

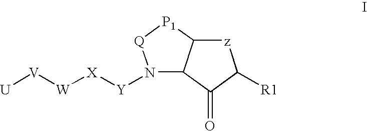

where UVWXY broadly corresponds to the P3 and P2 of dipeptide cysteine protease inhibitors, Z is inter alia O, S, methylene or —NR—, R¹ is alkyl, alkylaryl etc and P1 and Q1 are each methylene, optionally substituted with various carbon chains and cyclic groups. The compounds are alleged to be useful for the treatment of protozoal infections such as trypanosomes. There is no specific disclosure of haloalkyl isosteres of the P2/P3 amide bond.

We have now discovered that introduction of a halogen atom at a particular ring position in conjunction with halogenation of the P3/P2 linkage produces potent inhibitors of cathepsin K.

### BRIEF DESCRIPTION OF THE INVENTION

In accordance with the invention, there is provided compounds of the formula II

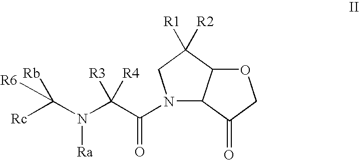

wherein
one of R¹ and R² is halo and the other is H or halo;
R³ is —C₁-C₅ straight or branched chain, optionally fluorinated, alkyl or —CH₂CR⁵C₃-C₄-cycloalkyl;

### R⁴ is H;

R⁵ is H, C₁-C₂ alkyl, C₁-C₂ haloalkyl, hydroxyl, OC₁-C₂alkyl, fluoro;
R⁶ is a stable, optionally substituted, monocyclic or bicyclic, carbocycle or heterocycle wherein the or each ring has 4, 5 or 6 ring atoms and 0 to 3 hetero atoms selected from S, O and N and wherein the optional substituents comprise 1 to 3 members selected from R₇;
R₇ is independently selected from halo, oxo, nitrile, nitro, C₁-C₄ alkyl, —XNRdRe, —XNReR⁸, —NReXR⁸, NH₂CO—, X—R⁸, X—O—R⁸, O—X—R⁸, X—C(═O)R⁸, X—(C═O)NRdR⁸, X—NReC(═O)R⁸, X—NHSOₘR⁸, X—S(═O)ₘR⁸, X—C(═O)OR⁸, X—NReC(═O)OR⁸;
R⁸ is independently H, C₁-C₄ alkyl, C₃-C₆ cycloalkyl, pyrrolidinyl, piperidinyl, morpholinyl, thiomorpholinyl, piperazinyl, indolinyl, pyranyl, thiopyranyl, furanyl, thienyl, pyrrolyl, oxazolyl, isoxazolyl, thiazolyl, imidazolyl, pyridinyl, pyrimidinyl, pyrazinyl, indolyl, phenyl, any of which is optionally substituted with up to 3 members selected from R⁹;
R⁹ is independently selected from hydroxy, XR¹⁰, —XNRdRe, —XNReR¹⁰, —NReC₁-C₄alkylR¹⁰, cyano, —S(═O)ₘRe, carboxy, oxo, C₁-C₄ alkyl, C₁-C₄ haloalkyl, C₁-C₄-alkoxy, C₁-C₄ alkanoyl, carbamoyl;
R¹⁰ is C₃-C₆ cycloalkyl, pyrrolidinyl, piperidinyl, morpholinyl, thiomorpholinyl, piperazinyl, indolinyl, pyranyl, thiopyranyl, furanyl, thienyl, pyrrolyl, oxazolyl, isoxazolyl, thiazolyl, imidazolyl, pyridinyl, pyrimidinyl, pyrazinyl, indolyl, phenyl, any of which is substituted with C₁-C₄ alkyl, halo, hydroxy, C₁-C₄alkoxy
X is independently a bond or C₁-C₄ alkylene;
Ra is independently H, C₁-C₄ alkyl or CH₃C(═O);
Rb is C₁-C₄haloalkyl;
Rc is H, C₁-C₄ alkyl;
Rd is independently H, C₁-C₄ alkyl or CH₃C(═O);
Re is independently H, C₁-C₄alkyl; or
Rd and Re together with the N atom to which they are attached form a morpholine, piperidine, piperazine or pyrrolidine ring optionally substituted with R⁹;
m is independently 0, 1 or 2;
or a pharmaceutically acceptable salt, hydrate or N-oxide thereof.

Without in any way wishing to be bound by theory, or the ascription of tentative binding modes for specific variables, P1, P2 and P3 as used herein are provided for convenience only and have their conventional meanings and denote those portions of the inhibitor believed to fill the S1, S2 and S3 subsites respectively of the enzyme, where S1 is adjacent the cleavage site and S3 remote from the cleavage site.

Preferably the stereochemistry of the P1 group is as depicted in the partial structure below:

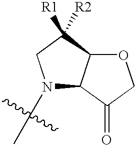

Preferably the halogen of R¹ and/or R² is chlorine and most preferably fluorine. It is currently preferred that R² is halo, especially fluorine and R¹ is H, but the invention extends to compounds wherein R¹ is halo, especially F and R² is H or R¹ and R² are each F.

It will be appreciated that the P1 group may exist in alternative forms, such as

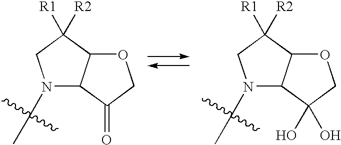

and the invention extends to all such alternative forms.

Preferably the stereochemistry of the P2 group corresponds to an L-amino acid as depicted in the partial structure below:

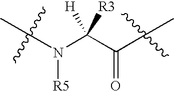

but the invention also extends to D-isomers.

The invention also includes all isomers and enantiomers at other chiral centres.

Currently preferred P2 groups include those wherein R⁴ is H and wherein R³ is iso-butyl or homo-t-butyl, that is —CH₂C(CH₃)₃, as shown below:

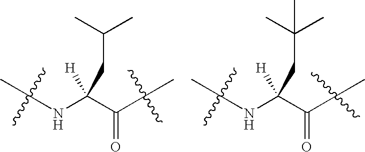

Further embodiments for R³ when R⁴ is H include those with the partial structure:

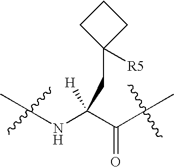

where R⁵ is as defined above.

Conveniently, R⁵ is H, thus defining a cyclobutylmethyl side chain at P2.

Representative values for R⁵ include methyl, hydroxyl, fluoromethyl, difluoromethyl or trifluoromethyl: Accordingly, favoured values of the P2 side chain include,

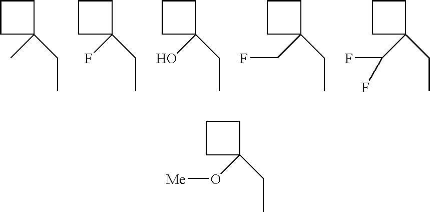

particularly those reflecting an L amino acid.

Currently preferred P2 groups include

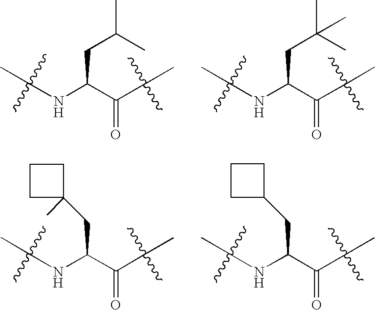

It is currently preferred that the Ra depicted in formula II is H.

Preferred haloalkyl groups for Rb include halomethyl such as fluoromethyl, difluoromethyl and preferably trifluoromethyl.

Typically Rc is H and R⁶ is a freestanding ring system as shown in the partial structure:

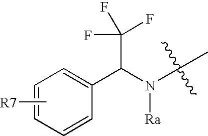

where R⁶ for the sake of illustration is exemplified with a substituted phenyl and Rb is trifluoromethyl:

Preferably the compound of the invention comprises a high enantiomeric purity, such as more than 80%, preferably more than 95% such as greater than 97% of the S stereoconfiguration at the carbon bearing haloalkyl Rb. The partial structure below represents a typical S-enantiomer with Rb as trifluoromethyl and Rc as H:

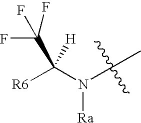

Returning now to formula II, Typically R⁶ is a monocyclic ring with 5 or especially 6 ring atoms, or a bicyclic ring structure comprising a 6 membered ring fused to a 4, 5 or 6 membered ring.

Typical R⁶ groups include saturated or unsaturated heterocycles or saturated or unsaturated carbocycles, any of which are optionally substituted as described above. Illustrative variants include C₃₋₈ cycloalkyl, phenyl, benzyl, tetrahydronaphthyl, indenyl, indanyl, heterocyclyl such as from azepanyl, azocanyl, pyrrolidinyl, piperidinyl, morpholinyl, thiomorpholinyl, piperazinyl, indolinyl, pyranyl, tetrahydropyranyl, tetrahydrothiopyranyl, thiopyranyl, furanyl, tetrahydrofuranyl, thienyl, pyrrolyl, oxazolyl, isoxazolyl, thiazolyl, imidazolyl, pyridinyl, pyrimidinyl, pyrazinyl, pyridazinyl, tetrazolyl, pyrazolyl, indolyl, benzofuranyl, benzothienyl, benzimidazolyl, benzthiazolyl, benzoxazolyl, benzisoxazolyl, quinolinyl, tetrahydroquinolinyl, isoquinolinyl, tetrahydroisoquinolinyl, quinazolinyl, tetrahydroquinazolinyl and quinoxalinyl, any of which may be substituted as described above.

The saturated heterocycle thus includes radicals such as pyrrolinyl, pyrrolidinyl, pyrazolinyl, pyrazolidinyl, piperidinyl, morpholinyl, thiomorpholinyl, pyranyl, thiopyranyl, piperazinyl, indolinyl, azetidinyl, tetrahydropyranyl, tetrahydrothiopyranyl, tetrahydrofuranyl, hexahydropyrimidinyl, hexahydropyridazinyl, 1,4,5,6-tetrahydropyrimidinylamine, dihydro-oxazolyl, 1,2-thiazinanyl-1,1-dioxide, 1,2,6-thiadiazinanyl-1,1-dioxide, isothiazolidinyl-1,1-dioxide and imidazolidinyl-2,4-dione, whereas the unsaturated heterocycle include radicals such as furanyl, thienyl, pyrrolyl, oxazolyl, thiazolyl, imidazolyl, pyrazolyl, isoxazolyl, isothiazolyl, oxadiazolyl, triazolyl, tetrazolyl, thiadiazolyl, pyridinyl, pyridazinyl, pyrimidinyl, pyrazinyl, indolizinyl, indolyl, isoindolyl. In each case the heterocycle may be condensed with a phenyl ring to form a bicyclic ring system.

Preferred monocyclic R⁶ groups include substituted pyridyl, substituted pyrimidyl, substituted phenyl, particularly phenyl substituted with a cyclic group such as pyrrolidine-1-yl, piperidine-1-yl, 4-methylpiperidin-1-yl, 4-(piperidin-3-ylmethyl)-piperidin-1-yl, morpholin-4-yl, 4-methylpiperazin-1-yl, 2-morpholin-4-yl-ethylamino, 2-morpholin-4-yl-ethyloxy, 1-pyrid-2-ylmethylamino, piperazin-1-yl, piperid-4-yl or N-piperazinyl, N-substituted with Ra or piperidin-1-yl which is 4-substituted with —NRaRb. A phenyl R⁶ is conveniently substituted at the 3 or 4 position (para or meta), for example with such a cyclic group.

Alternative cyclic substituents to a monocyclic R⁶ (such as phenyl) include aryl groups such as phenyl or a 5 or 6 membered heteroaryl group such as thiophene, furyl, triazole, thiazole, diazole, pyrazole or pyrrolidine. Favoured cyclic substituents in this context include thiazol-2-yl, pyrid-3-yl and especially pyrid-2-yl, thien-2-yl or thiazol-5-yl. This cyclic substituent (i.e. R⁷) is typically bonded direct to such R⁶ species (i.e. X is a bond), but may also for example comprise an amine spacer such as —NH—, —N(Me), —CH₂NH, —CH₂N(Me)—, a C₁-C₃alkyl spacer such as —CH₂— or a C₁-C₃-alkyloxy spacer such as ethyloxy

Any of the cyclic substituents to R⁶ in the immediately preceding paragraph may be substituted as described above with R¹⁰. For example a heterocycle R⁷ group such as thiazolyl can be substituted with C₁-C₄ alkyl such as methyl.

Preferably, any of the cyclic substituents to R⁶ in the two immediately preceding paragraphs may itself be substituted with a cyclic group (that is R⁷ comprises an R⁹ moiety) typically a saturated heterocyclic group such as piperidine, piperazine or morpholine, which saturated cyclic group is optionally substituted, for example with C₁-C₃ alkyl, fluoro, difluoro, C₁-C₃alkyloxy or C₁-C₃alkyloxyC₁-C₃alkyl. As provided in the definition of R⁷, this saturated cyclic group (i.e. R⁹) may be spaced from the R⁶ group by X (e.g. C₁-C₃alkyl), amine (e.g. —NH—), amide, sulphonamide etc, but is typically bonded directly or via methylene.

Representative R⁹ groups in accordance with the immediately preceding paragraph include heterocycles such as pyrrolidine-1-yl, piperidine-1-yl, 4-methylpiperidin-1-yl, 4-(piperidin-3-ylmethyl)-piperidin-1-yl, morpholin-4-yl, 4-methylpiperazin-1-yl, 2-morpholin-4-yl-ethylamino, 2-morpholin-4-yl-ethyloxy, 1-pyrid-2-ylmethylamino, piperazin-1-yl, piperid-4-yl or N-piperazinyl, N-substituted with Ra or piperidin-1-yl which is 4-substituted with —NRaRb,

Currently preferred R⁹ substituents include 4-substituted piperazin-4-yl, such as 4-methyl-piperazin-4-yl or 4-methyloxyethyl-piperazin-4-yl, piperid-1-ylmethyl which is optionally 4-substituted with fluoro or difluoro or morpholinylmethyl.

Alternative preferred substituents to a monocyclic R⁶ (such as phenyl) include —NRaRb, —CH₂NRaRb, C₁-C₄ straight or branched alkyl or —O—R⁹.

Alternative preferred substituents to a monocyclic R⁶ (such as phenyl) include groups include non-basic moieties, such as halo, hydroxyl, carboxy, C₁-C₄haloalkyl, C₁-C₄ alkyloxy, and those of the formula:

—S(═O)ₘC₁-C₄ alkyl, —S(═O)ₘC₃-C₆ cycloalkyl, or a carbamoyl substituted cycloalkyl group with the partial structure:

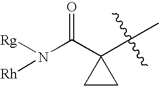

where Rg is H C₁-C₄ alkyl or cyclopropyl, Rh is H, C₁-C₄ alkyl; or Rg and Rh together with the N atom to which they are attached define pyrrolidine, morpholine, piperidine, piperazine or N-methylpiperazine.

Representative R⁶ groups include:

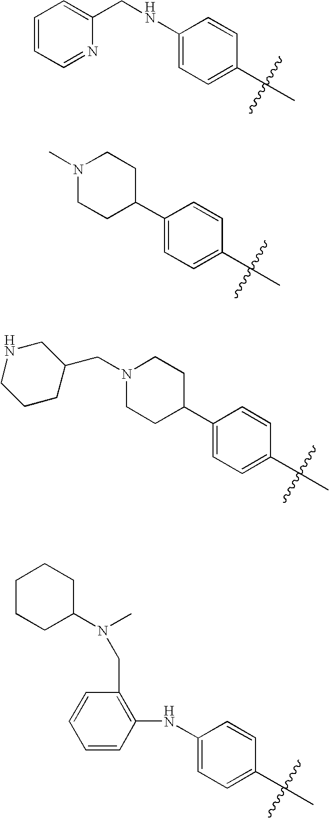

Further representative R⁶ groups include

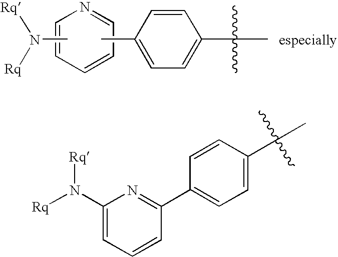

where Rq and Rq′ are independently selected from H, C₁-C₄ alkyl or C₁-C₄alkanoyl or together define an unsaturated 5-7 membered ring, such as piperidine, piperazine or morpholine, which may in turn be substituted with groups corresponding to R¹⁰, particularly C₁-C₄ alkyl, fluoro or difluoro.

Currently preferred R⁶ groups include

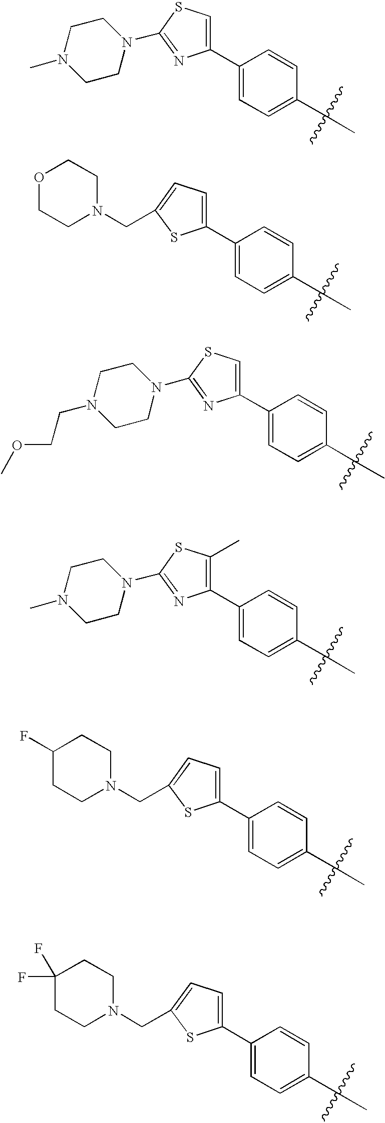

Representative bicyclic groups for R⁶ include naphthylenyl, especially naphthylen-2-yl; benzo[1,3]dioxolyl, especially benzo[1,3]dioxol-5-yl, benzofuranyl, especially benzofuran-2-yl, and especially C₁-C₆ alkoxy substituted benzofuranyl, more especially 5-(2-piperazin-4-carboxylic acid tert-butyl ester-ethoxy)benzofuran-2-yl, 5-(2-morpholino-4-yl-ethoxy)-benzofuran-2-yl, 5-(2-piperazin-1-yl-ethoxy)benzofuran-2-yl, 5-(2-cyclohexyl-ethoxy)-benzofuran-2-yl; 7-methoxy-benzofuran-2-yl, 5-methoxy-benzofuran-2-yl, 5,6-dimethoxy-benzofuran-2-yl, especially halogen substituted benzofuranyl, more especially 5-fluoro-benzofuran-2-yl, 5,6-difluoro-benzofuran-2-yl, especially C₁-C₆alkyl substituted benzofuranyl, most especially 3-methyl-benzofuran-2-yl; benzo[b]thiophenyl, especially benzo[b]thiophen-2-yl; especially C₁-C₆alkoxy substituted benzo[b]thiopheny], more especially 5,6-dimethoxy-benzo[b]thiophen-2-yl, quinolinyl, especially quinolin-2-yl, quinolin-3-yl, quinolin-4-yl, quinolin-6-yl, and quinolin-5-yl; quinoxalinyl, especially quinoxalin-2-yl; 1,8-naphthyridinyl, especially 1,8-naphthyridin-2-yl; indolyl, especially indol-2-yl, especially indol-6-yl, indol-5-yl, especially C₁-C₆alkyl substituted indolyl, more especially N-methylindol-2-yl; furo[3,2-b]pyridinyl, especially furo[3,2-b]pyridin-2-yl, and C₁-C₆-alkyl substituted furo[3,2-b]pyridinyl, especially 3-methyl-furo[3,2-b]pyridin-2-yl; thieno[3,2-b]thiophene, especially thieno[3,2-b]thiophene-2-yl, more especially C₁-C₆alkyl substituted thieno[3,2-b]thiophene-2-yl, more especially 5-tert-buty]-3-methylthieno[3,2-b]thiophene-2-yl.

Favoured R⁶ groups include bicyclic rings such as napthyl, quinoloyl, benzofuranyl, benzothienyl, indolyl and indolinyl, particularly where the linkage is to the 2 position of the ring. Favoured substituents to a bicyclic R⁶ group include pyrrolidine-1-yl, piperidine-1-yl, 4-methylpiperidin-1-yl, 4-(piperidin-3-ylmethyl)-piperidin-1-yl, morpholin-4-yl, 4-methylpiperazin-1-yl, 2-morpholin-4-yl-ethylamino, 2-morpholin-4-yl-ethyloxy, 1-pyrid-2-ylmethylamino, piperazin-1-yl, piperid-4-yl or N-piperazinyl, N-substituted with Ra or piperidin-1-yl which is 4-substituted with —NRaRb. Especially preferred substituents, particularly in conjunction with benzofuranyl include 2-morpholin-4-yl-ethyloxy and N-methyl-piperidin-4-yloxy and those defined below.

A currently favoured bicyclic R⁶ group is optionally substituted benzothiazol or benzofuryl or benzoxazolyl, including those wherein the substituent is —OR⁹ or —NRbR⁹. For example, favoured R⁶ groups include benzofur-2-yl, unsubstituted or substituted at the 3 position with C₁-C₄ alkyl, such as methyl or C₁-C₄ haloalkyl such as trifluoromethyl and/or substituted in the 5 position with a saturated heterocycle such as piperidine, piperazine or morpholine, which is optionally substituted with C₁-C₃ alkyl and/or spaced from the benzofuryl by oxy, methyloxy or ethyloxy. Particularly favoured benzofuryl R⁶ groups thus include:

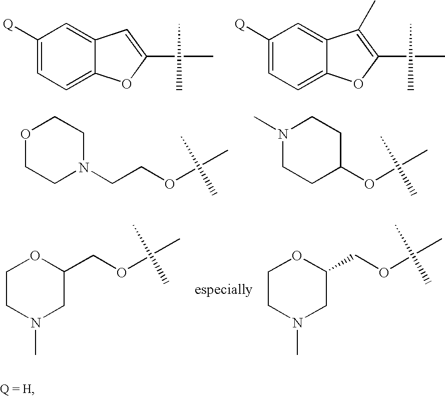

Returning to formula II in general:

X is typically methylene or especially a bond.

C₁-Cₙ alkyl, where n is 4, on its own or within compound expressions such as C₁-C₄ alkoxy, includes methyl, ethyl, n-propyl, iso-propyl, n-butyl, iso-butyl, sec-butyl, t-butyl, cyclopropyl, methylcyclopropyl and the like, extended in a likewise fashion for other values of n. For example C₅ alkyl includes homo-t-butyl (—CH₂C(CH₃)₃).

Halogen or halo includes bromo, chloro and especially fluoro.

Haloalkyl means an alkyl group as defined above where at least one carbon atom bears 1 to 3 halogen atoms, preferably fluorine atoms. Representative haloalkyl groups include fluoromethyl, difluoromethyl, trifluoromethyl, 2, fluoroethyl, 2,2difluorethyl, 2,2,2 trifluorethyl and the like.

Favoured compounds of the invention include those permutations formed by independent selection of a P3, P2 and P1 member from each of Tables A, B and C:

TABLE A
P1 groups

| | | |
|---|---|---|
| 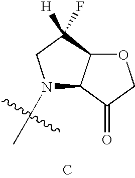 | 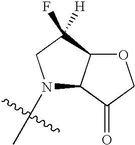 | 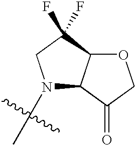 |

TABLE B
P2 groups

| | |
|---|---|
| 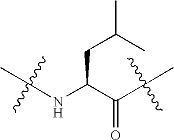 | 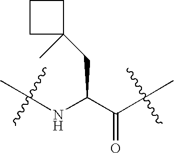 |

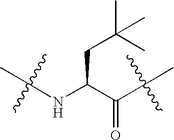

TABLE C
P3 groups

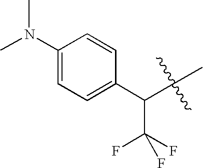

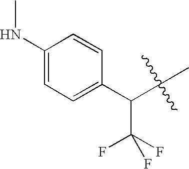

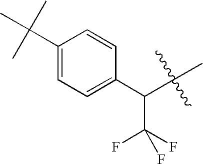

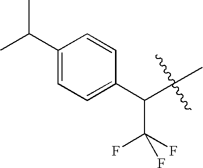

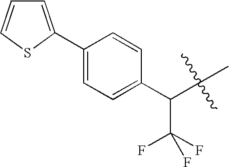

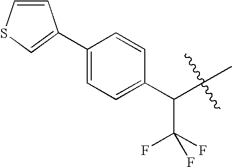

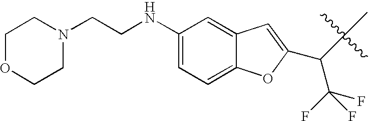

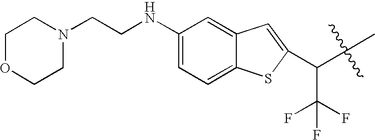

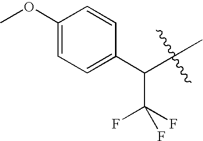

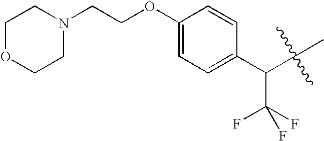

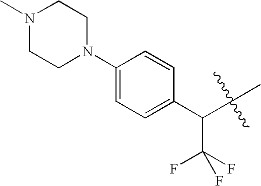

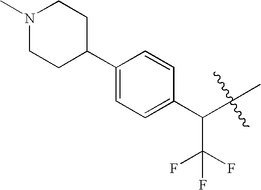

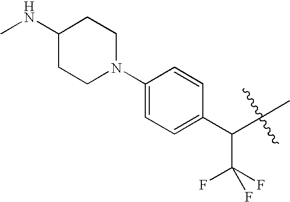

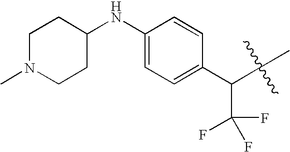

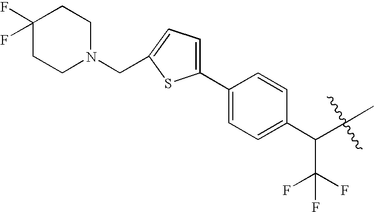

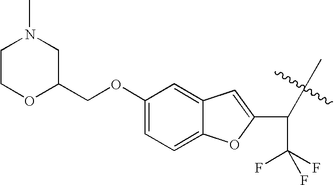

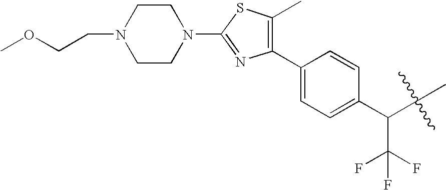

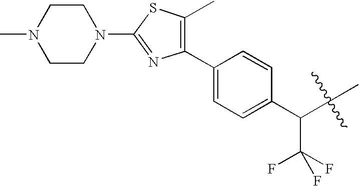

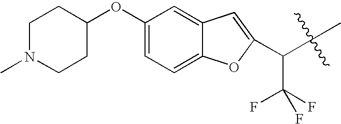

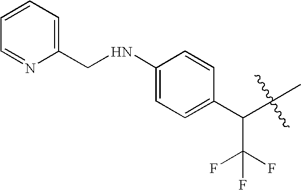

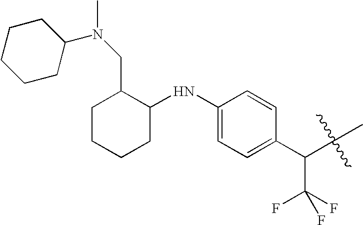

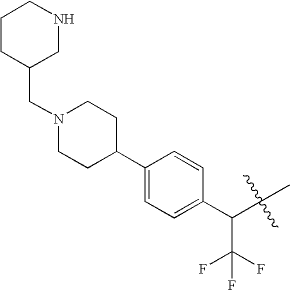

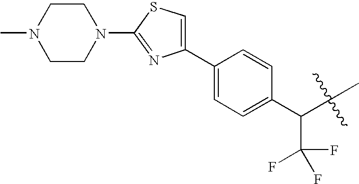

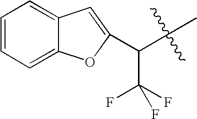

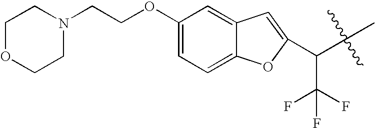

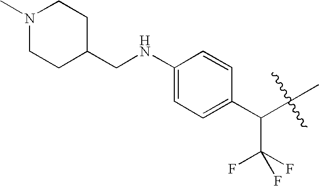

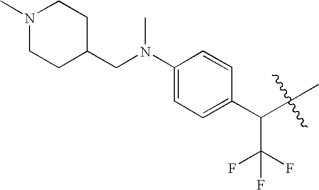

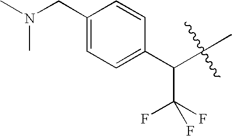

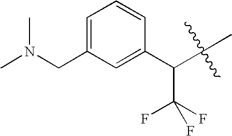

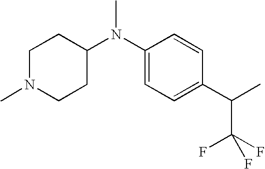

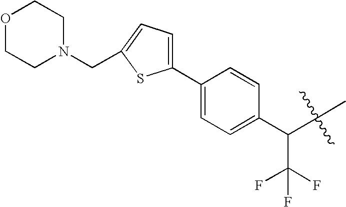

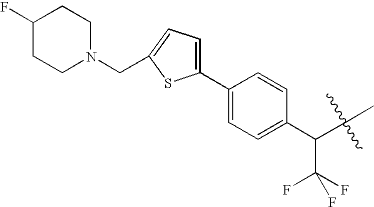

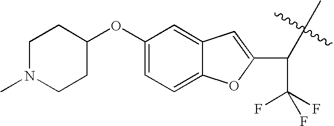

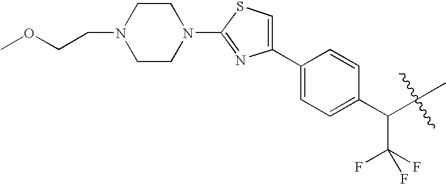

Additional aspects of the invention include a pharmaceutical composition comprising a compound as defined above and a pharmaceutically acceptable carrier or diluent therefor.

A further aspect of the invention is the use of a compound as defined above in the manufacture of a medicament for the treatment of disorders mediated by cathepsin K, such as:
osteoporosis,
gingival diseases such as gingivitis and periodontitis,
Paget's disease,
hypercalcaemia of malignancy
metabolic bone disease
diseases characterised by excessive cartilage or matrix degradation,
such as osteoarthritis and rheumatoid arthritis,
bone cancers including neoplasia,
pain.

The invention is believed to be of particular utility against osteoporosis, osteoarthritis, rheumatoid arthritis and/or bone metastases.

The compounds of the invention can form salts which form an additional aspect of the invention. Appropriate pharmaceutically acceptable salts of the compounds of Formula II include salts of organic acids, especially carboxylic acids, including but not limited to acetate, trifluoroacetate, lactate, gluconate, citrate, tartrate, maleate, malate, pantothenate, isethionate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-napthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids. The compounds of Formula II may in some cases be isolated as the hydrate. Hydrates are typically prepared by recrystallisation from an aqueous/organic solvent mixture using organic solvents such as dioxin, tetrahydrofuran or methanol.

The N-oxides of compounds of Formula (I) can be prepared by methods known to those of ordinary skill in the art. For example, N-oxides can be prepared by treating an unoxidized form of the compound of Formula (I) with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds of Formula (I) can be prepared from the N-oxide of an appropriate starting material.

Compounds of Formula (I) in unoxidized form can be prepared from N-oxides of compounds of Formula (I) by treating with a reducing agent (e.g., sulfur, sulfur dioxide, triphenyl phosphine, lithium borohydride, sodium borohydride, phosphorus bichloride, tribromide, or the like) in an suitable inert organic solvent (e.g., acetonitrile, ethanol, aqueous dioxane, or the like) at 0 to 80° C.

Compounds of Formula (II) can be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diasteromeric derivatives of compounds of Formula (I), dissociable complexes are preferred (e.g., crystalline; diastereoisomeric salts). Diastereomers have distinct physical properties (e.g., melting points, boiling points, solubilities, reactivity, etc.) and can be readily separated by taking advantage of these dissimilarities. The diastereomers can be separated by chromatography, for example HPLC or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet, Samuel H. Wilen, Enantiomers, Racemates and Resolutions, John Wiley & Sons, Inc. (1981).

It will be appreciated that the invention extends to prodrugs, solvates, complexes and other forms releasing a compound of formula II in vivo.

While it is possible for the active agent to be administered alone, it is preferable to present it as part of a pharmaceutical formulation. Such a formulation will comprise the above defined active agent together with one or more acceptable carriers/excipients and optionally other therapeutic ingredients. The carrier(s) must be acceptable in the sense of being compatible with the other ingredients of the formulation and not deleterious to the recipient.

The formulations include those suitable for rectal, nasal, topical (including buccal and sublingual), vaginal or parenteral (including subcutaneous, intramuscular, intravenous and intradermal) administration, but preferably the formulation is an orally administered formulation. The formulations may conveniently be presented in unit dosage form, e.g. tablets and sustained release capsules, and may be prepared by any methods well known in the art of pharmacy.

Such methods include the step of bringing into association the above defined active agent with the carrier. In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound of Formula II or its pharmaceutically acceptable salt in conjunction or association with a pharmaceutically acceptable carrier or vehicle. If the manufacture of pharmaceutical formulations involves intimate mixing of pharmaceutical excipients and the active ingredient in salt form, then it is often preferred to use excipients which are non-basic in nature, i.e. either acidic or neutral.

Formulations for oral administration in the present invention may be presented as discrete units such as capsules, cachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion and as a bolus etc.

With regard to compositions for oral administration (e.g. tablets and capsules), the term suitable carrier includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring or the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

The appropriate dosage for the compounds or formulations of the invention will depend upon the indication and the patient and is readily determined by conventional animal trials. Dosages providing intracellular (for inhibition of physiological proteases of the papain superamily) concentrations of the order 0.01-100 μM, more preferably 0.01-10 μM, such as 0.1-25 μM are typically desirable and achievable.

Compounds of the invention are prepared by a variety of solution and solid phase chemistries.

The compounds are typically prepared as building blocks reflecting the P1, P2 and P3 moieties of the end product inhibitor. Without in any way wishing to be bound by theory, or the ascription of tentative binding modes for specific variables, the notional concepts P1, P2 and P3 as used herein are provided for convenience only and have substantially their conventional Schlecter & Berger meanings and denote those portions of the inhibitor believed to fill the S1, S2, and S3 subsites respectively of the enzyme, where S1 is adjacent the cleavage site and S3 remote from the cleavage site. Compounds defined by Formula I are intended to be within the scope of the invention, regardless of binding mode.

Broadly speaking the P1 building block will be an N-protected-6-fluoro-3-oxo-hexahydro-furo[3,2-b]pyrrole, P2 will be an N-protected amino acid, whereas P3 typically comprises a capping group such as a substituted, heteroaroyl or aroyl moiety linked to P2 via the Rb-haloalkyl substituted carbon linkage.

The suitably protected individual building blocks can first be prepared and subsequently coupled together i.e. P2+P1→P2-P1. Alternatively, precursors of the building blocks can be coupled together and modified at a later stage of the synthesis of the inhibitor sequence. Further building blocks, precursors of building blocks or prefabricated bigger fragments of the desired structure, can then be coupled to the growing chain, e.g. R³-E-P2*+P1→R³-E-P2-P¹ or R³-E*+P2-P1→R³-E-P2-P1, where * denotes an activated form.

Formation of a peptide bond, i.e. coupling can be carried out using standard coupling procedures such as the azide method, mixed carbonic-carboxylic acid anhydride (isobutyl chloroformate) method, carbodiimide (dicyclohexylcarbodiimide, diisopropylcarbodiimide, or water-soluble carbodiimide) method, active ester (p-nitrophenyl ester, N-hydroxysuccinic imido ester) method, Woodward reagent K-method, carbonyldiimidazole method, phosphorus reagents or oxidation-reduction methods. Some of these methods (especially the carbodiimide method) can be enhanced by adding 1-hydroxybenzotriazole or 4-DMAP. These coupling reactions can be performed in either solution (liquid phase) or solid phase.

More explicitly, the coupling step involves the dehydrative coupling of a free carboxyl of one reactant with the free amino group of the other reactant in the present of a coupling agent to form a linking amide bond. Descriptions of such coupling agents are found in general textbooks on peptide chemistry, for example, M. Bodanszky, “Peptide Chemistry”, 2nd rev ed., Springer-Verlag, Berlin, Germany, (1993) hereafter simply referred to as Bodanszky, the contents of which are hereby incorporated by reference. Examples of suitable coupling agents are N,N′-dicyclohexylcarbodiimide, 1-hydroxybenzotriazole in the presence of N,N′-dicyclohexylcarbodiimide or N-ethyl-N′-[(3-dimethylamino) propyl]carbodiimide. A practical and useful coupling agent is the commercially available (benzotriazol-1-yloxy)tris-(dimethylamino)phosphonium hexafluorophosphate, either by itself or in the present of 1-hydroxybenzotriazole or 4-DMAP. Another practical and useful coupling agent is commercially available 2-(1H-benzotriazol-1-yl)-N,N,N′,N′-tetramethyluronium tetrafluoroborate. Still another practical and useful coupling agent is commercially available 0-(7-azabenzotrizol-1-yl)-N,N,N′,N′-tetramethyluronium hexafluorophosphate.

The coupling reaction is conducted in an inert solvent, e.g. dichloromethane, acetonitrile or dimethylformamide. An excess of a tertiary amine, e.g. diisopropylethylamine, N-methylmorpholine, N-methylpyrrolidine or 4-DMAP is added to maintain the reaction mixture at a pH of about 8. The reaction temperature usually ranges between 0° C. and 50° C. and the reaction time usually ranges between 15 min and 24 h.

The functional groups of the constituent non-natural amino acids generally must be protected during the coupling reactions to avoid formation of undesired bonds. The protecting groups that can be used are listed in Greene, “Protective Groups in Organic Chemistry”, John Wiley & Sons, New York (1981) and “The Peptides: Analysis, Synthesis, Biology”, Vol. 3, Academic Press, New York (1981), hereafter referred to simply as Greene, the disclosures of which are hereby incorporated by reference.

The alpha-carboxyl group of the C-terminal residue is usually protected as an ester that can be cleaved to give the carboxylic acid. Protecting groups that can be used include 1) alkyl esters such as methyl, trimethylsilyl and t.butyl, 2) aralkyl esters such as benzyl and substituted benzyl, or 3) esters that can be cleaved by mild base or mild reductive means such as trichloroethyl and phenacyl esters.

The alpha-amino group of each amino acid to be coupled is typically N-protected. Any protecting group known in the art can be used. Examples of such groups include: 1) acyl groups such as formyl, trifluoroacetyl, phthalyl, and p-toluenesulfonyl; 2) aromatic carbamate groups such as benzyloxycarbonyl (Cbz or Z) and substituted bensyloxycarbonyls, and 9-fluorenylmethyloxycarbonyl (Fmoc); 3) aliphatic carbamate groups such as tertbutyloxycarbonyl (Boc), ethoxycarbonyl, diisopropylmethoxycarbonyl, and allyloxycarbonyl; 4) cyclic alkyl carbamate groups such as cyclopentyloxycarbonyl and adamantyloxycarbonyl; 5) alkyl groups such as triphenylmethyl and benzyl; 6) trialkylsilyl such as trimethylsilyl; and 7) thiol containing groups such asphenylthiocarbonyl anddithiasuccinoyl. The preferred alpha-amino protecting group is either Boc or Fmoc. Many amino acid derivatives suitably protected for peptide synthesis are commercially available.

The alpha-amino protecting group is typically cleaved prior to the next coupling step. When the Boc group is used, the methods of choice are trifluoroacetic acid, neat or in dichloromethane, or HCl in dioxane or in ethyl acetate. The resulting ammonium salt is then neutralized either prior to the coupling or in situ with basic solutions such as aqueous buffers, or tertiary amines in dichloromethane or acetonitrile or dimethylformamide. When the Fmoc group is used, the reagents of choice are piperidine or substituted piperidine in dimethylformamide, but any secondary amine can be used. The deprotection is carried out at a temperature between 0° C. and room temperature usually 20-22° C.

Any of the natural or non-natural amino acids having side chain functionalities will typically be protected during the preparation of the peptide using any of the above described groups. Those skilled in the art will appreciate that the selection and use of appropriate protecting groups for these side chain functionalities depend upon the amino acid and presence of other protecting groups in the peptide. In the selection of such protecting groups it is desirable that the group is not removed during the deprotection and coupling of the alpha-amino group.

For example, when Boc is used as the alpha-amino protecting group, the following side chain protecting groups are suitable: p-toluenesulfonyl (tosyl) moieties can be used to protect the amino side chain of amino acids such as Lys and Arg; acetamidomethyl, benzyl (Bn), or tert-butylsulfonyl moities can be used to protect the sulfide containing side chain of cysteine; benzyl (Bn) ethers can be used to protect the hydroxy containing side chains of serine, threonine or hydroxyproline; and benzyl esters can be used to protect the carboxy containing side chains of aspartic acid and glutamic acid.

When Fmoc is chosen for the alpha-amine protection, usually tert. butyl based protecting groups are acceptable. For instance, Boc can be used for lysine and arginine, tert.butyl ether for serine, threonine and hydroxyproline, and tert-butyl ester for aspartic acid and glutamic acid. Triphenylmethyl (Trityl) moiety can be used to protect the sulfide containing side chain of cysteine.

Once the inhibitor sequence is completed any protecting groups are removed in whatever manner is dictated by the choice of protecting groups. These procedures are well known to those skilled in the art.

The first stage in a synthesis of compounds of the general formula II is typically the preparation in solution of a functionalized P1 building block. Different nomenclature of compounds according to the present invention can be used. For convenience the carbohydrate nomenclature will generally be used herein. A typical scheme towards a bicyclic P1 group starts with the ring closure of a suitably protected intermediate which is available in 4 steps from 1,2:5,6-di-O-isopropylidene-D-allofuranose, described by Mayer zum Reckendorf, Chem. Ber. 101 (1968), 3802-3807, giving a precursor of 3S, 4R stereochemistry.

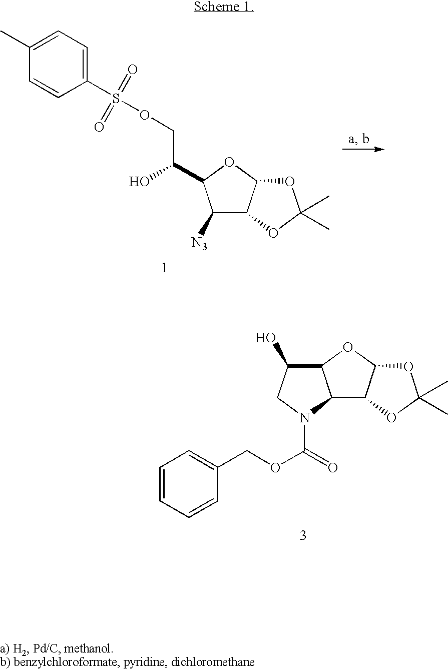

In Scheme 1 the azide group of derivative 1 is reduced for example by catalytic hydrogenation using palladium on charcoal or other catalysts suitable, in a suitable solvent such as an alcohol, like ethanol or methanol into the free amine. The obtained nucleophilic nitrogen reacts spontaneously, or optionally in the presence of a suitable base like such as triethyl amine or sodium acetate, with the C-6 position forming a 5,5-bicycle. The leaving group at C-6 is not limited to sulfonate esters, but also other leaving groups such as halogen could be used throughout the synthesis of compounds according to the present invention. The reduction of the azide residue into an amine could also be performed by other methods known from literature, such as treating the azide derivative with a trialkyl- or triarylphosphine followed by hydrolysis of the formed imine derivative. After the ring closure the amine may be N-protected with a suitable protecting group such as a carbamate, like benzyl carbamate of compound 3 or any other similar protecting group which is normally not cleaved with acid. Suitable protecting groups which can be found in: Protective groups in organic chemistry, 3^{rd} edition, 1999, Theodora W. Greene and Peter G. M. Wuts (Wiley&sons).

For a 3R, 4S bicycle a similar approach could be used starting from 3-azido-3-deoxy-1,2:5,6-di-O-isopropylidene-D-gulofuranose which can be prepared as described in Tetrahedron Asymmetry, 10 (1999) 1855-1859. This intermediate can then be treated as described in Scheme 2.

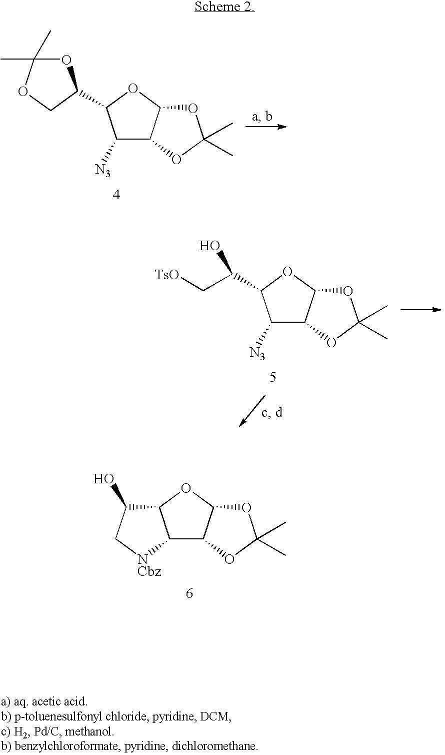

Compound 4 can be treated with a mild acid, such as diluted acetic acid or similar, which can selectively hydrolyze the 5,6-acetal of compound 4, to obtain a diol. The primary alcohol can be selectively reacted with an alkyl- or arylsulfonyl chloride like p-toluenesulfonyl chloride to give compound 5. The azide group of derivative 5 is reduced for example by catalytic hydrogenation using palladium on charcoal or other catalysts suitable, in a suitable solvent such as an alcohol, like ethanol or methanol into the free amine. The obtained nucleophilic nitrogen reacts spontaneously, or optionally in the presence of a suitable base like such as triethyl amine or sodium acetate, with the C-6 position forming a 5,5-bicycle which can be N-protected with a suitable protecting group such as its benzyl carbamate (Cbz) to give compound 6. Alternatively 3-azido-3-deoxy-1,2:5,6-di-O-isopropylidene-D-idofuranose (Bull. Chem. Soc. Japan, 57, 1 (1984), 237-241) could be a suitable starting material for the 3R, 4S bicycle according to Scheme 3.

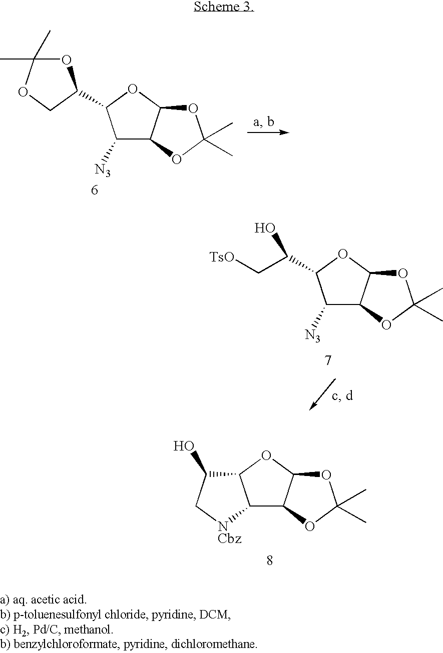

Compound 6 can be treated with a mild acid, such as diluted acetic acid or similar, which can selectively hydrolyze the 5,6-acetal of compound 6, to obtain a diol. The primary alcohol can be selectively reacted with an alkyl- or arylsulfonyl chloride like p-toluenesulfonyl chloride to give compound 7. The azide group of derivative 7 is reduced for example by catalytic hydrogenation using palladium on charcoal or other catalysts suitable, in a suitable solvent such as an alcohol, like ethanol or methanol into the free amine. The obtained nucleophilic nitrogen reacts spontaneously, or optionally in the presence of a suitable base like such as triethyl amine or sodium acetate, with the C-6 position forming a 5,5-bicycle which can be N-protected with a suitable protecting group such as its benzyl carbamate (Cbz) to give compound 8.

The ring closure is not limited to the substrates shown above but could also be applied to derivatives as depicted in Scheme 4.

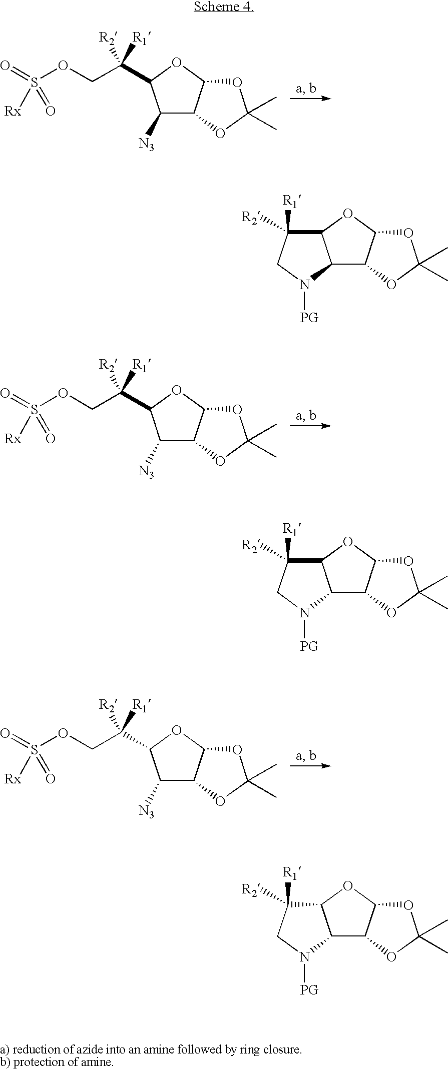

Rx in Scheme 4 may be chosen from methyl, trifluoromethyl, p-methylphenyl or similar residues present in readily available alkylsulfonylhalides, preferably a bulky Rx suitable for regioselective reaction on the primary alcohol of a diol as described in Chem. Ber. 101 (1968), 3802-3807. R^{1′} and R^{2′} are R¹ and R² as defined. Pg could be a suitable protecting group such as a carbamate, like benzyl carbamate or any similar protecting group which is not normally cleaved with acid.

Further substrates for the ring closure reaction could be compounds depicted in Scheme 5.

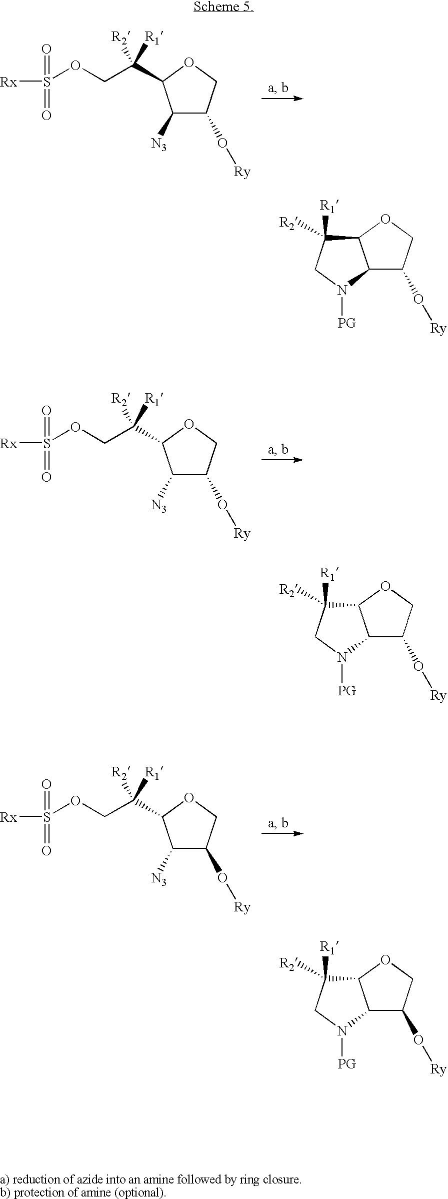

Rx in Scheme 5 can be chosen from methyl, trifluoromethyl, p-methylphenyl or similar residues present in readily available alkylsulfonylhalides, preferably a bulky Rx suitable for regioselective reaction on the primary alcohol of a diol as described in Chem. Ber. 101 (1968), 3802-3807. R^{1′} and R^{2′} are R¹ and R² as defined above. Ry can be hydrogen or a hydroxylprotective group, preferably an ether type protective group. Preferably Ry is hydrogen. PG could be a suitable N-protecting group such as a carbamate, for derivatives in Scheme 5, Ry is typically hydrogen.

Other methodologies to obtain a 5,5-bicycle is disclosed by G. Lin and Z. Shi, Tetrahedron, 53, 4, 1369-1382, 1997.

Further modification of the 5,5-bicyclic compound obtained in scheme 1 is outlined in Scheme 6.

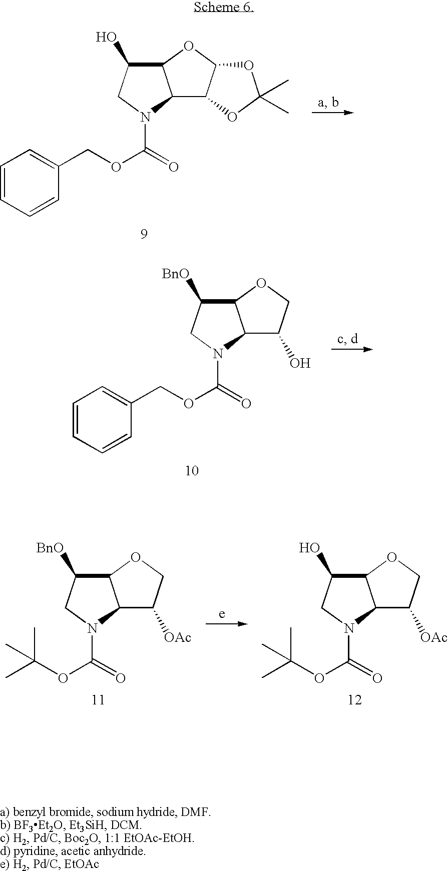

Compound 9 is protected with a suitable acid stable protecting group such as substituted methyl ether, in particular a benzyl ether, by treating the mono-ol 9 with a base such as sodium hydride or sodium hydroxide in an aprotic solvent such as N,N-dimethylformamide (DMF) in the presence of the desired alkylating agent such as the benzyl halide, in particular benzyl bromide. The obtained material can then be reduced into compound 10 according to methods described by G. J. Ewing and M. J. Robins, Org. Lett. 1, 4, 1999, 635-636, or by references therein. Preferably the reduction is performed with excess boron trifluoride etherate in the presence of a reducing agent such as trialkylsilane, in particular with excess triethylsilane in a suitable non-protic solvent such as dichloromethane. Catalytic hydrogenation of compound 10 using for example palladium-on-charcoal in a suitable solvent or solvent mixture such as ethyl acetate-ethanol in a hydrogen atmosphere, in the presence of di-tert-butyl dicarbonate followed by treatment of the product with acetic anhydride in pyridine gives intermediate 11. By repeated catalytic hydrogenation, as described above, the mono-ol 12 is obtained.

A fluorine can be introduced on compound 12, and the bicyclic compound then N-deprotected according to Scheme 7.

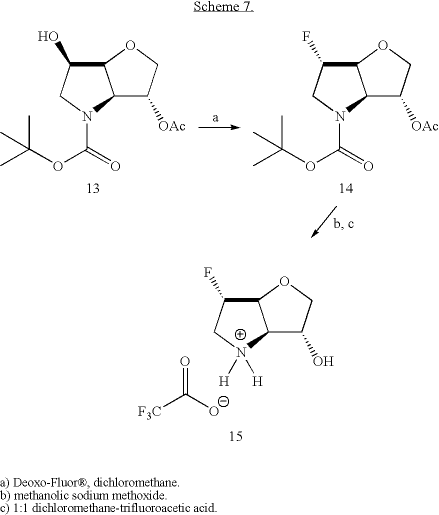

Compound 13 can be treated with a fluorinating agent such as [bis-(2-methoxyethyl)aminosulfur trifluoride] (Deoxo-Fluor®) or with similar fluorinating agents such as diethylaminosulfur trifluoride (DAST) which gives the product 14 with inversion of configuration at C-5. Compound 14 is then deacetylated by treatment for example with methanolic sodium methoxide, or any similar alkaline solutions with an inorganic base such as sodium hydroxide or sodium carbonate, followed by N-deprotection using acidic conditions such as dichloromethane-trifluoroacetic acid solutions or other methods which could be found in: Protective Groups in Organic Chemistry, 3^{rd} edition, 1999, Theodora W. Greene and Peter G. M. Wuts (Wiley & Sons).

Alternatively the epimeric fluorine can be obtained by treating derivative 9 above according to Scheme 8.

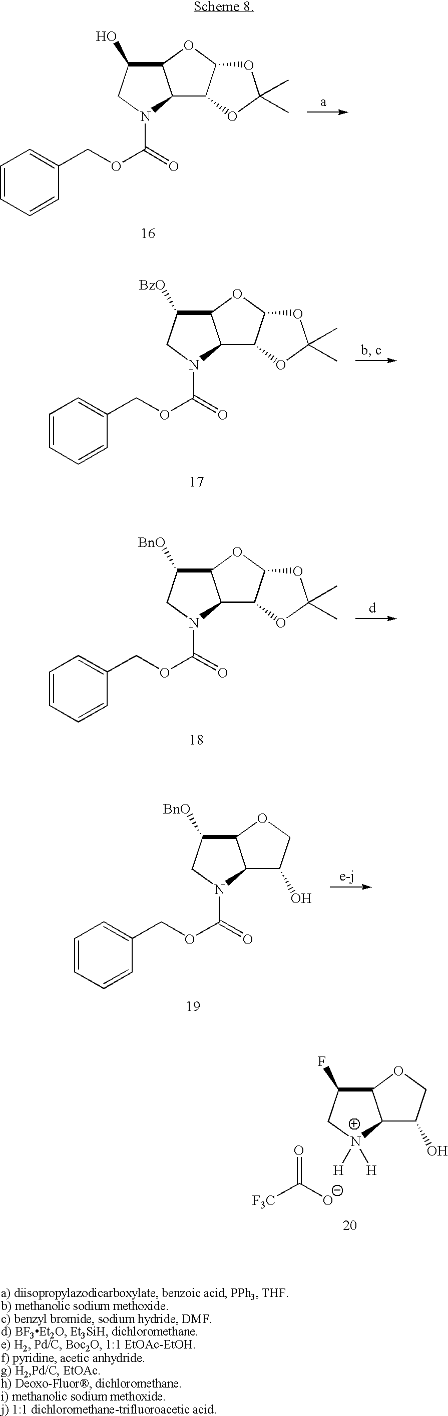

Inversion of configuration at C-5 can be accomplished by reacting compound 16 under Mitsunobo conditions which gives a benzoate ester. Ester hydrolysis with methanolic sodium methoxide followed by treatment of the mono-ol with benzyl bromide provides benzyl protected epimer 17. Reaction steps d-j in Scheme 8 are as described for Schemes 6 and 7.

A further route to a “difluoro derivative” wherein R¹ and R² are fluoro is shown in Scheme 9.

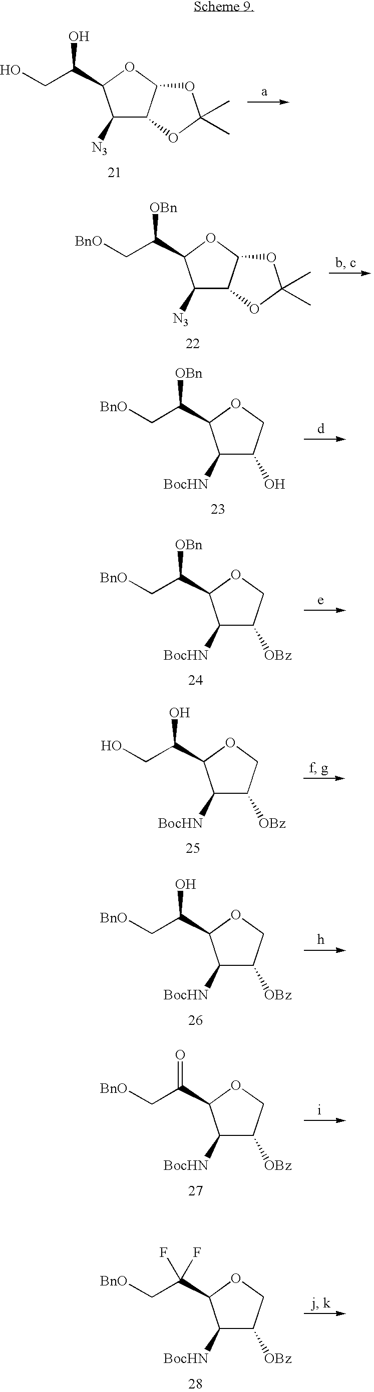

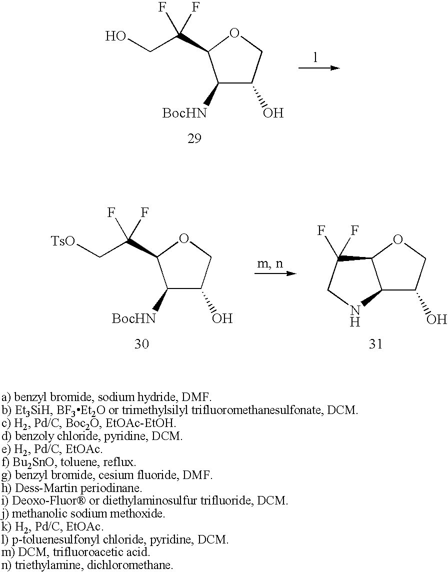

The synthesis of the P1 building block can be started from compound 21 (3-azido-3-deoxy-1,2-O-isopropylidene-D-allofuranose) which is described by Mayer zum Reckendorf, Chem. Ber. 101 (1968), 3802-3807. Treatment of compound 21 with a benzylating agent like benzyl bromide or benzyl chloride in the presence of a base, such as sodium hydride or sodium hydroxide in a aprotic polar solvent, such as N,N-dimethylformamide gives derivative 22. Compound 22 is then treated with a trialkyl silane, such as triethyl silane, with an excess of a Lewis acid such as boron trifluoride etherate or trimethylsilyl trifluoromethanesulfonate, in a aprotic solvent such as dichloromethane. The resulting azide can then be selectively reduced by catalytic hydrogenation using for example Palladium on charcoal in the presence of di-tert-butyl carbonate to obtain compound 23. Alternatively the azide could be reduced with other methods known from literature such as triphenylphosphine-water, followed by protection giving a suitable carbamate. In order to avoid problems with regioselectivity in the following steps, compound 23 could be treated with an acylating agent such as an acyl chloride or acid anhydride, such as benzoyl chloride, in neat organic base such as pyridine or triethyl amine, or in a mixture of an aprotic solvent such as dichloromethane and a base to give compound 24. Catalytic hydrogenation of compound 24 as described above gives diol 25. Selective benzylation at the primary alcohol of compound 25 can be accomplished by several methods known from the literature. In Scheme 9 the diol is refluxed with dibutyl tin oxide in a suitable solvent such as toluene to form a tin acetal. The tin acetal can then be reacted with a small excess of benzyl bromide and cesium fluoride in DMF giving the desired compound 26. Oxidation of 26 with a suitable oxidizing agent such as Dess-Martin periodinane in dichloromethane converts the secondary alcohol into the keto compound 27 suitable to convert into the difluoride 28. This can be accomplished by treating compound 27 with an excess fluorinating agent such as Deoxo-Fluor®, or with diethylaminosulfur trifluoride (DAST), in an aprotic solvent such as dichloromethane or 1,2-dichloroethane. The benzoate ester of compound 28 can be cleaved with alkali such as methanolic sodium methoxide, followed by debenzylation using catalytic hydrogenation to obtain diol 29. Selective introduction of a sulfonate ester at the primary alcohol can be accomplished by treating the compound 29 with a small excess of alkyl- or arylsulfonyl chloride in the presence of a base such as pyridine in suitable solvent such as dichloromethane, adding the sulfonylating agent at reduced temperature and slowly increase up to room temperature, which gives mono-ol 30. Treatment of compound 30 under acidic conditions such as mixtures of dichlormethane-trifluoroacetic acid liberates the amine, and treating the product with a base such as triethyl amine promotes the internal ring closure which gives building block 31.

Alternative routes to 5,5-bicycles are shown in Schemes 10 and 11.

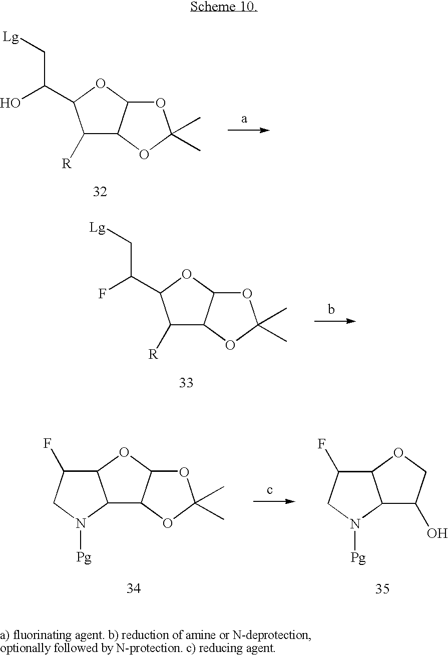

In Scheme 10 a derivative such as compound 32 (available as described above or with methods well known in the art) with the substituents at C-3 and C-4 in cis relationship, Lg being a leaving group such as halogen or a sulfonate ester, and with R equal to an azide or a nitrogen protected with a suitable N-protecting group, can be treated with a fluorinating agent such as mentioned above, producing compound 33. Upon liberating the masked amine with either reduction of the azide or by a suitable deprotection method, the amine could perform an intramolecular attack at C-6 producing a 5,5-bicycle with structure 34, which could optionally be N-protected (Pg=protecting group or hydrogen). Reduction of C-1 with a suitable reducing agent such as described above or with a similar reducing agent would give building block 35.

In Scheme 11 an alternative route to a difluoro-5,5-bicycle is depicted.

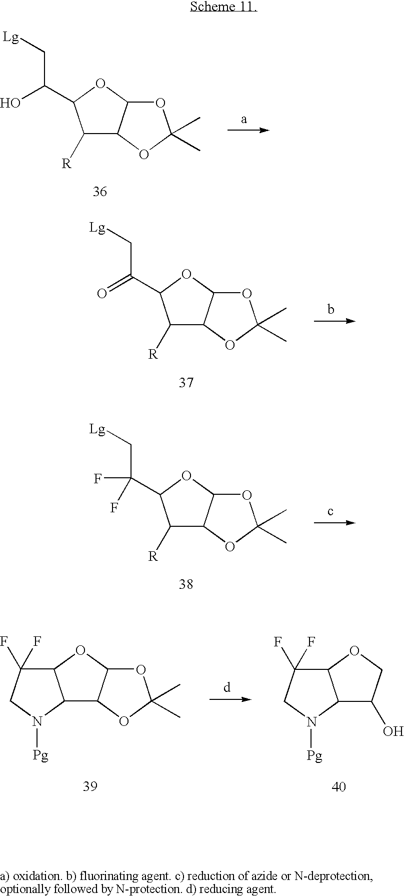

In Scheme 11 compound 36 (available as described above or with methods well known in the art) with the substituents at C-3 and C-4 in cis relationship, Lg being a leaving group such as halogen or a sulfonate ester, and with R equal to an azide or a nitrogen protected with a suitable protecting group, can be oxidized with a Swern-type reaction or other suitable methods which can give compound 37. Treatment of compound 37 according to Scheme 11 with an excess of fluorinating agent such as mentioned above, gives compound 38. Upon liberating the masked amine of 38 with either reduction of the azide or by a suitable deprotection method, the amine could perform an intramolecular attack at C-6 producing a 5,5-bicycle with structure 39, which could optionally be N-protected (Pg=protecting group or hydrogen). Reduction of C-1 with a suitable reducing agent such as described above or with a similar reducing agent gives building block 40.

A convenient route to compounds wherein R¹ or R² is a halogen such as chloro is depicted in Scheme 12

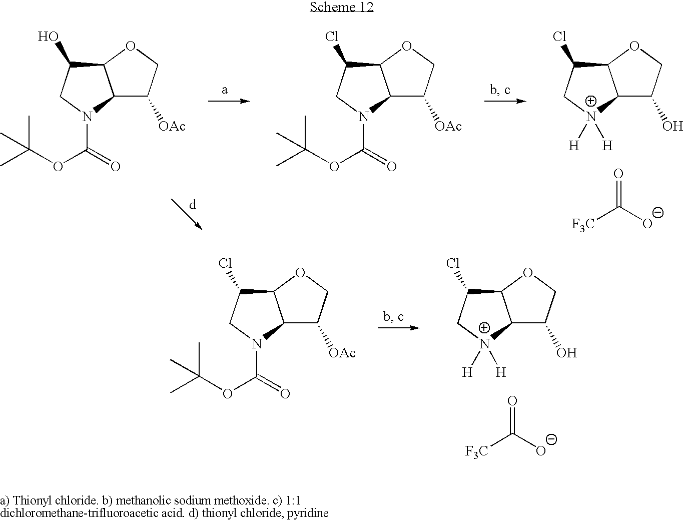

The P1 building block is typically elongated with the natural or non natural P2 amino acid (or the P3+P2 building block) by conventional solution or solid phase chemistries, such as those outlined or exemplified below, or disclosed in WO00/69855 or WO02/057270. P2 and P3 groups are either commercially available as enantiomers or resolvable from the racemate or obtainable using simple chemical transformations known to one skilled in the art. For example, 4-(methyl-piperazine-1-yl)-benzoic acid can be obtained using Buchwald chemistry (S. L. Buchwald & J. P. Wolfe, Journal of Organic Chemistry, 2000, 65, 1144) and subsequently elaborated. Other P3 cores such as 4-(1-piperidin-4-yl)-benzoic acid are prepared from 1-(4-phenyl-piperidine-1-yl)-ethanone using a Friedel-Crafts acylation reaction and subsequently elaborated using standard chemical transformations known to one skilled in the art. Alternatively, other P3 moieties, such as 5-[2-(4-morpholinyl)ethoxy]-2-benzofuran-2-carboxylic acid, are prepared using Mitsunobu reactions on solid phase as detailed by L. S. Richter & T. R. Gadek in Tetrahedron Lett., 1994, 35, 4705.

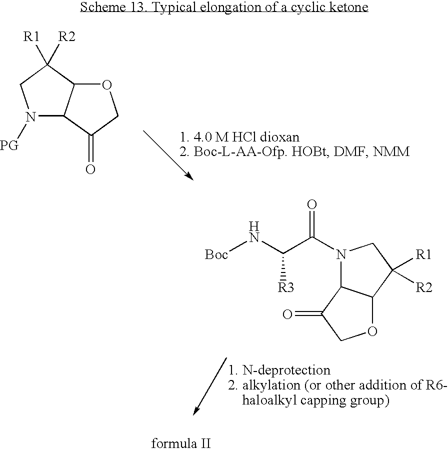

Alternatively the P1 building block as the hydroxyl may be elongated and subsequently oxidised as shown in Scheme 14.

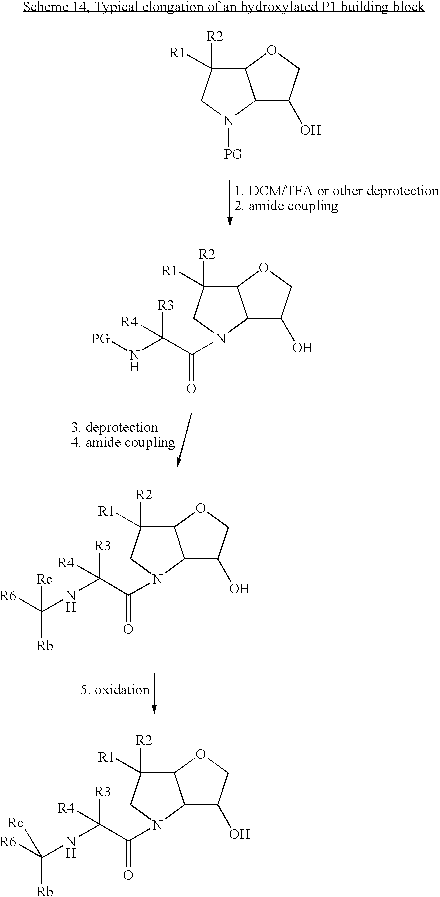

The P3 cap is typically elongated by reaction of an intermediate compound of the formula

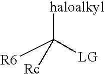

where R⁶ and Rc are as defined above and LG is a conventional leaving group such as trifluoromethansulfonate, and the like, with the N-deprotected P1/P2 building block shown above. The reaction is carried out in a suitable organic solvent, including but not limited to, halogenated organic solvents such as methylene chloride, 1,2-dibromoethane, and the like, ethereal solvents such as diethyl ether, tetrahydrofuran, acetonitrile, or aromatic solvents such as benzene, toluene, xylene, and the like, or mixtures thereof and optionally in the presence of an organic or inorganic base. Preferably, the organic base is triethylamine, pyridine, N-methylmorpholine, collidine, diisopropylethylamine, and the like. Preferably, the inorganic base is cesium carbonate, sodium carbonate, sodium bicarbonate, and the like. The reaction is optionally carried out in the presence of a drying agent such as molecular sieves. Preferably, the reaction is carried out at room temperature. The intermediate can be prepared by methods well known in the art. For example, a compound where R⁶ is phenyl or 4-fluorophenyl, Rb is trifluoromethyl, and Rc is hydrogen can be readily prepared from commercially available 2,2,2 trifluoroacetophenone or 2,2,2,4′-tetrafluoroacetophone respectively, by reducing the keto group to an alcoholic group by suitable reducing agent such as sodium borohydride, lithium aluminum hydride, and the like. The solvent used depends on the type of reducing agent. For example, when sodium borohydride is used the reaction is carried out in an alcoholic organic solvent such as methanol, ethanol, and the like. When lithium aluminum hydride is used the reaction is carried out in an ethereal solvent such as tetrahydrofuran, and the like. Reaction of 2,2,2 trifluoro-1-phenylethanol or 222-trifluoro-1-(4-fluorophenyl)ethanol with triflic anhydride provides the desired compound. Chirally enriched intermediate can be obtained by reduction of the corresponding halogenated acetophenone with a suitable reducing agent such as catecholborane or BH₃-DMS complex in the presence of a suitable catalyst such as (A or (R) CBS catalyst or (A or (R)-, a-diphenyl-2-pyrrolidine-methanol in the presence of BBN.

In a corresponding fashion, the intermediate of the formula:

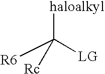

can be reacted with the carboxy-protected P2 building block, which is subsequently deprotected and elongated with the P1 building block as described herein.

Alternatively the above described intermediate is reacted:

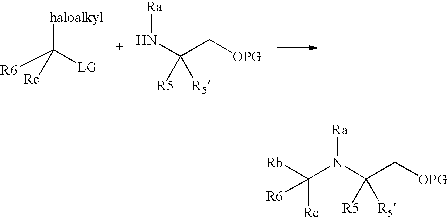

LG is a suitable leaving group such as trifluoromethansulfonate, and PG a suitable hydroxyl protecting group such as trialkylsilyl, and the like, under the reaction conditions described above. The resulting O-protected hydroxyethylamide is oxidised to the corresponding carboxylic acid and couple to the P1 building block as described below. Suitable hydroxyl protecting groups and reaction conditions for putting them on and removing them can be found in Greene, T. W.; and Wuts, P. G. M. Protecting Groups in Organic Synthesis; John Wiley & Sons, Inc. 1999. The P2 hydroxyethylamine can be prepared from the corresponding natural and unnatural amino acids by methods well known in the art. Some such procedures are described in PCT Application Publication No. WO 03/075836, the disclosure of which is incorporated herein by reference in its entirety.

Alternatively compounds wherein E is —CRbRc— can be prepared by reaction of a compound of the formula

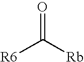

where R⁶ is a cyclic group as defined above and Rb is halomethyl, preferably trifluoromethyl with the N-deprotected, carboxy-protected P2 building block or the P1/P2 building block outlined above under reductive amination reaction conditions. The reaction is carried out in the presence of a suitable dehydrating agent such as TiCl₄, magnesium sulfate, isopropyl trifluoroacetate, in the presence of a base such as diisopropylethylamine, pyridine, and the like and in a suitable organic solvent such as methylene chloride to give an imine. The imine is reduced with a suitable reducing agent such as sodium borohydride, sodium cyanoborohydride, and the like in a suitable organic solvent such as methanol, ethanol, and the like.

Alternatively compounds wherein E is —CRbRc— can be prepared by reaction of the haloalkylaldehyde with an amine as shown below:

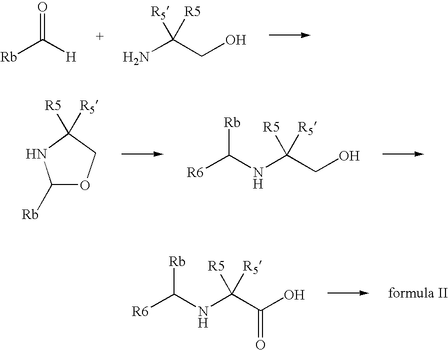

R⁵, R^{5′}, R6 and Rb are as defined above. Condensation of the haloalkylaldehyde with an aminoethanol (prepared by reducing the corresponding 5/5′ alpha amino acid with a suitable reducing agent such as lithium aluminum hydride, and the like under conditions well known in the art), utilizing Dean Stark apparatus provides the depicted cyclic aminal which upon reaction with a Grignard reagent of formula R⁶MgX (where X is halo) or an organolithium reagent of formula R⁶Li I provides the depicted hydroxyethylamide. Oxidation of the hydroxyethylamide with a suitable oxidizing agent such as Jones oxidizing reagent or H₅IO₆/CrO₃, and the like, then provides the P3/P2 building block which is C-terminal elongated with the P1 building block and oxidised as necessary.

As described above elongation is typically carried out in the presence of a suitable coupling agent e.g., benzotriazole-1-yloxytrispyrrolidinophosphonium hexafluorophosphate (PyBOP), O-benzotriazol-1-yl-N,N,N′,N′-tetramethyl-uronium hexafluorophosphate (HBTU), 0-(7-azabenzotriazol-1-yl)-1,1,3,3-tetramethyl-uronium hexafluorophosphate (HATU), 1-(3-dimethylaminopropyl)-3-ethylcarbodiimide hydrochloride (EDC), or 1,3-dicyclohexyl carbodiimide (DCC), optionally in the presence of 1-hydroxybenzotriazole (HOBT), and a base such as N,N-diisopropylethylamine, triethylamine, N-methylmorpholine, and the like. The reaction is typically carried out at 20 to 30° C., preferably at about 25° C., and requires 2 to 24 h to complete. Suitable reaction solvents are inert organic solvents such as halogenated organic solvents (e.g., methylene chloride, chloroform, and the like), acetonitrile, N,N dimethylformamide, ethereal solvents such as tetrahydrofuran, dioxane, and the like.

Alternatively, the above elongation coupling step can be carried out by first converting the P3/P2 building block into an active acid derivative such as succinimide ester and then reacting it with the P1 amine. The reaction typically requires 2 to 3 h to complete. The conditions utilized in this reaction depend on the nature of the active acid derivative. For example, if it is an acid chloride derivative of 4, the reaction is carried out in the presence of a suitable base (e.g. triethylamine, diisopropylethylamine, pyridine, and the like). Suitable reaction solvents are polar organic solvents such as acetonitrile, N,N-dimethylformamide, dichloromethane, or any suitable mixtures thereof.

The above method can also be used to prepared compounds where Rc is other than hydrogen utilizing the procedure described above, but substituting R⁶COH with a ketone of formula R⁶RbCO and then treating the resulting cyclic aminal with RcLi/RcMgX, followed by oxidation to give the free acid. The free acid is then condensed with under conditions described above.

It will be apparent to a person skilled in the art, that compounds wherein E is CRbRc can also be prepared as follows:

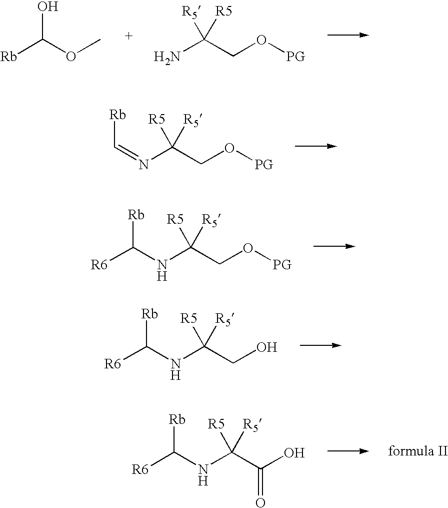

In particular conventional O-protection of the above described aminoethanol followed by reaction with the haloalkylhemiacetal provides the depicted haloalkylimine compound, which is treated with an organic lithium compound of formula R⁶Li where R⁶ is as defined above. Removal of the oxygen protecting group provides the hydroxyethyamide described in the immediately preceding scheme above which in the corresponding fashion is oxidised to the carboxylic acid and elongated with the P1 building block. Suitable oxygen protecting groups and reaction conditions for putting them on and removing them can be found in Greene, T. W.; and Wuts, P. G. M.; Protecting Groups in Organic Synthesis; John Wiley & Sons, Inc. 1999.

Alternatively, a compound wherein E is CRbRc and R⁶ is aryl or heteroaryl can be prepared as illustrated below:

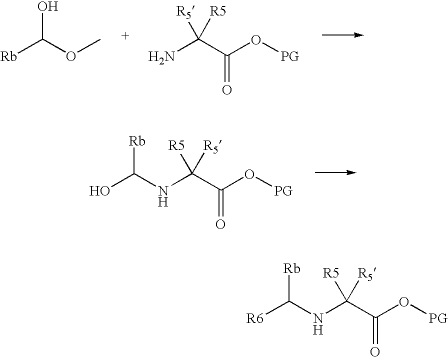

In particular the above described haloalkyl hemiacetal is reacted with the protected P2 building block to yield the depicted 2-(1-hydroxymethylamino)acetate intermediate. The reaction is carried out in the presence of a catalytic amount of an acid such as p-toluenesulfonic acid and in an aromatic hydrocarbon solvent such as toluene, benzene, and the like.

Treatment of the 2-(1-hydroxymethylamino)acetate intermediate with R⁶H under Friedel-Crafts reaction conditions/BF₃.EtT₂O provides the carboxy protected P3/P2 building block which is elongated as described above. Similarly, the haloalkyhemiacetal can be reacted with the P2/P1 building block, and oxidised to the ketone, as necessary.

The term “N-protecting group” or “N-protected” as used herein refers to those groups intended to protect the N-terminus of an amino acid or peptide or to protect an amino group against undesirable reactions during synthetic procedures. Commonly used N-protecting groups are disclosed in Greene, “Protective Groups in Organic Synthesis” (John Wiley & Sons, New York, 1981), which is hereby incorporated by reference. N-protecting groups include acyl groups such as formyl, acetyl, propionyl, pivaloyl, t-butylacetyl, 2-chloroacetyl, 2-bromoacetyl, trifluoracetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl, and the like, carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butoxycarbonyl, diisopropylmethoxycarbonyl, isopropyloxycarbonyl, ethoxycarbonyl, methoxycarbonyl, allyloxycarbonyl, 2,2,2-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl, and the like; alkyl groups such as benzyl, triphenylmethyl, benzyloxymethyl and the like; and silyl groups such as trimethylsilyl and the like. Favoured N-protecting groups include formyl, acetyl, benzoyl, pivaloyl, t-butylacetyl, phenylsulfonyl, benzyl, t-butoxycarbonyl (BOC) and benzyloxycarbonyl (Cbz).

Hydroxy and/or carboxy protecting groups are also extensively reviewed in Greene ibid and include ethers such as methyl, substituted methyl ethers such as methoxymethyl, methylthiomethyl, benzyloxymethyl, t-butoxymethyl, 2-methoxyethoxymethyl and the like, silyl ethers such as trimethylsilyl (TMS), t-butyldimethylsilyl (TBDMS) tribenzylsilyl, triphenylsilyl, t-butyldiphenylsilyl triisopropyl silyl and the like, substituted ethyl ethers such as 1-ethoxymethyl, 1-methyl-1-methoxyethyl, t-butyl, allyl, benzyl, p-methoxybenzyl, dipehenylmethyl, triphenylmethyl and the like, aralkyl groups such as trityl, and pixyl (9-hydroxy-9-phenylxanthene derivatives, especially the chloride). Ester hydroxy protecting groups include esters such as formate, benzylformate, chloroacetate, methoxyacetate, phenoxyacetate, pivaloate, adamantoate, mesitoate, benzoate and the like. Carbonate hydroxy protecting groups include methyl vinyl, allyl, cinnamyl, benzyl and the like.

### DETAILED DESCRIPTION OF THE EMBODIMENTS

Various embodiments of the invention will now be described by way of illustration only with reference to the following Examples.

### Example 1

### Construction of P1 Building Block

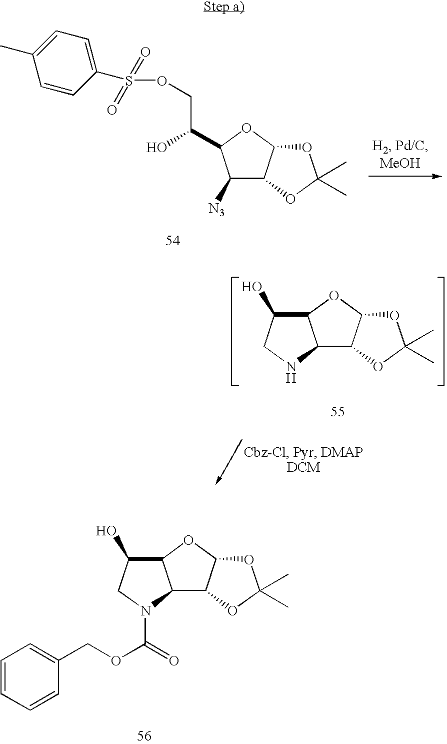

A mixture of 54 (5.2 g, 13.0 mmol), palladium-on-carbon (10%, Acros, 0.66 g) in methanol was hydrogenated at slight positive pressure. The hydrogen was changed 3 times over a period of 1 h, after TLC (petroleum ether-ethyl acetate 7:3 and dichloromethane-methanol 9:1, staining with ammonium molybdate-cerium sulfate) indicated complete conversion of the starting material into a major non-UV active spot which colours AMC, and some weaker higher moving spots (dichloromethane-methanol 9:1). The reaction mixture was then filtered through Celite and concentrated which gave crude compound 55.

To a suspension of the residue in dichloromethane (60 ml) and pyridine (3.2 ml, 40 mmol) at 0° C. was added benzylchloroformate (0.93 ml, 6.5 mmol). The reaction mixture was stirred at room temperature for 2 h after which additional pyridine (3 ml) and benzylchloroformate (0.8 ml) was added at 0° C. The reaction mixture was then stirred at room temperature overnight, then diluted with dichloromethane (100 ml), washed successively with 1M aq. sulfuric acid (2×50 ml) and 1M aq. sodium hydrogen carbonate (1×50 ml), then dried (sodium sulfate), filtered and concentrated onto silica. Flash chromatography (diameter: 4 cm, YMC-gel: 50 g, packing eluent: ethyl acetate in petroleum ether 1:4) of the residue using ethyl acetate in petroleum ether 1:4 (350 ml), 2:3 (250 ml), 1:1 (250 ml), 3:2 (250 ml) and 3:1 (150 ml) gave compound 56 as a foamy syrup (2.71 g, 8.1 mmol, 62% over 2 steps) after drying in vacuum overnight.

NMR data (400 MHz, CDCl₃): ¹H, 1.33, 1.52 (2 s, 6H, C(CH₃)₂), 2.34 (2 d, 1H, —OH), 3.04 (m, 1H, H-6a), 3.97 (m, 1H, H-6b), 4.19 (m, 1H, H-5), 4.33 (m, 1H, H-3), 4.68, 4.84 (2 d, 1H, H-2), 4.79 (t, 1H, H-4), 5.08-5.24 (m, 2H, CH₂Ph), 5.86 (br s, 1H, H-1), 7.30-7.42 (m, 5H, Ar—H).

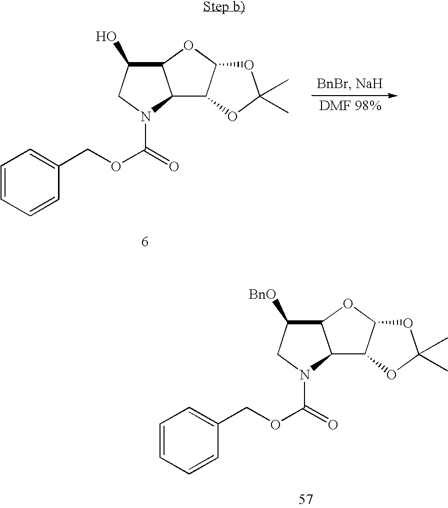

To a stirred suspension of sodium hydride (60% in mineral oil, Aldrich, 0.34 g, 8.4 mmol) and compound 56 (2.17 g, 6.47 mmol) in dimethylformamide (30 ml) was added benzyl bromide (0.81 mmol, 6.8 mmol) during 5 minutes. After stirring 1 h (TLC: ethyl acetate in petroleum ether 2:3), methanol (approx 2 ml) was added to destroy excess reagent, then immediately partitioned between ethyl acetate (180 ml) and water (150 ml). The organic layer was washed with water (3×100 ml), then dried (sodium sulfate), filtered and concentrated onto silica. Flash chromatography (diameter: 4 cm, YMC-gel: 40 g, packing eluent: ethyl acetate in petroleum ether 1:4) of the residue using ethyl acetate in petroleum ether 1:4 (100 ml), 3:7 (250 ml) and 2:3 (250 ml) gave a colourless syrup (2.7 g, 6.35 mmol, 98%) after drying in vacuum overnight.

NMR data (400 MHz, CDCl₃): ¹H, 1.31 (s, 3H, C(CH₃)(CH₃)), 1.51 (d, 3H, C(CH₃)(CH₃)), 3.29 (m, 1H, H-6a), 3.78-3.96 (m, 2H, H-5 and H-6b), 4.22 (dd, 1H, H-3), 4.64, 4.84 (2 M, 4H, H-2, H-4 and CH₂Ph), 5.07-5.22 (m, 1H, CH₂Ph), 5.94 (m, 1H, H-1), 7.28-7.39 (m, 10H, Ar—H).

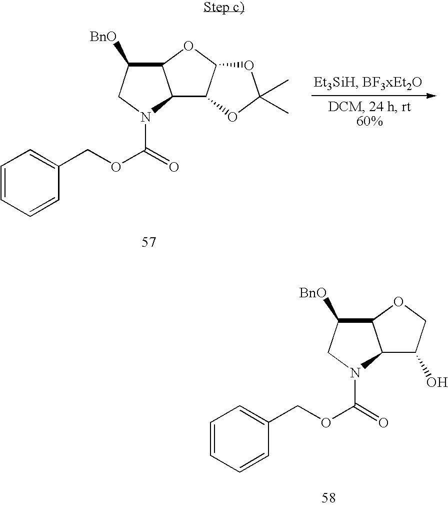

To a stirred solution of compound 7 (2.635 g, 6.19 mmol) in dichloromethane (28 ml) and triethyl silane (9.9 ml, 61.9 mmol) at 0° C. was added borontrifluoride etherate (7.9 ml, 61.9 mmol) in one portion. The reaction mixture was then stirred at rt for 24 h (TLC: petroleum ether-ethyl acetate 4:1 and ethyl acetate-toluene 3:2), then 1M aq. sodium hydrogen carbonate (40 ml) and some solid sodium hydrogen carbonate was carefully added until bubbling stopped. The resulting mixture was partitioned between dichloromethane (100 ml) and water (100 ml). The organic layer was washed with 1M aq. sodium hydrogen carbonate (1×100 ml) and brine (1×100 ml), then dried (sodium sulfate), filtered and concentrated onto silica. Flash chromatography (diameter: 4 cm, YMC-gel: 48 g, packing eluent: ethyl acetate-toluene 3:2) of the residue using ethyl acetate in toluene 3:2 (750 ml) gave a colorless hard syrup (1.38 g, 3.74 mmol, 60%) of about 85-90% purity according to TLC. LR-MS: Calcd for C₂₁H₂₄NO₅: 370.2. Found: 370.0 [M+H].

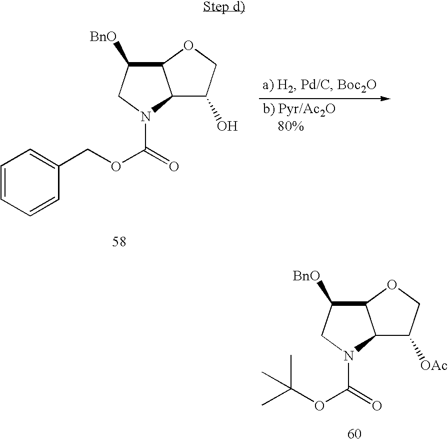

A mixture of compound 58 (1.38 g, 3.74 mmol), palladium-on-carbon (Acros, 10%, 0.12 g) and di-tert-butyl-dicarbonate (0.82 g, 3.7 mmol) in ethyl acetate (50 ml) was hydrogenated at slight overpressure. The hydrogen was changed 2 times over a period of 1 h and the reaction was monitored by LC-MS. After 1 h, additional palladium-on-carbon (0.1 g) was added and the reaction mixture was treated with hydrogen for 1 more hour. The reaction mixture was then filtered through Celite and concentrated. The residue was treated with 2:1 pyridine-acetic anhydride (18 ml) overnight, and then concentrated. The residue was redissolved in dichloromethane (60 ml) and was washed successively with 1M aq. sulfuric acid (2×40 ml) and 1M aq. sodium hydrogen carbonate (1×40 ml), and then dried (sodium sulfate) filtered and concentrated. Flash chromatography (diameter: 3 cm, YMC-gel: 20 g, packing eluent: ethyl acetate in toluene 1:4) of the residue (dissolved in toluene-ethyl acetate 4:1) using ethyl acetate in toluene 1:4 (200 ml) and 1:3 (150 ml) gave a colourless syrup (1.13 g, 3.0 mmol, 80%) after drying in vacuum overnight.

NMR data (400 MHz, CDCl₃): ¹H, 1.45 (s, 9H, C(CH₃)₃), 2.08 (s, 3H, COCH₃), 3.10 (m, 1H, H-6a), 3.74-3.99 (m, 3H, H-1a, H-5 and H-6b), 4.11 (m, 1H, H-1b), 4.16-4.74 (m, 4H H-3, H-4 and CH₂Ph), 5.31 (m, 1H, H-2), 7.28-7.40 (m, 5H, Ar—H).

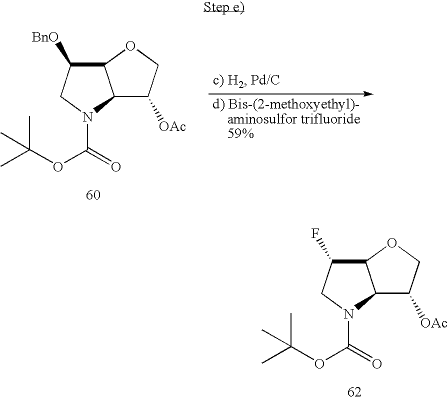

A mixture of compound 60 (1.08 g, 2.86 mmol) and palladium-on-carbon (10%, 0.15 g) in ethyl acetate (30 ml) was hydrogenated at slight over pressure for 2 h (TLC: toluene-ethyl acetate 4:1 and 1:1), then filtered through Celite and concentrated. The mixture was concentrated from dichloromethane (3×10 ml), then dissolved in dichloromethane and to the solution was added bis-(2-methoxyethyl)aminosulphurtrifluoride (50% in THF, 2.12 ml, 2 eq.) at 0° C. After stirring at rt overnight additional bis(2-methoxyethyl)aminosulphurtrifluoride (50% in THF, 2 ml) was added and the reaction mixture was stirred at rt for another night (TLC: toluene-ethyl acetate 1:1, ninhydrine staining), then 1M aq. sodium hydrogen carbonate was added carefully until bubbling stopped. The resulting mixture was diluted with dichloromethane (50 ml), and the organic layer was washed once with 1 M aq. sodium hydrogen carbonate (40 ml), then dried (sodium sulfate), filtered and concentrated. Flash chromatography (diameter: 3 cm, Silica: 25 g, packing eluent: toluene-ethyl acetate 4:1) of the residue (dissolved in toluene-ethyl acetate 4:1) using toluene-ethyl acetate 4:1 gave compound 62 (0.49 g, 1.7 mmol, 59%) as a colourless syrup after drying in vacuum overnight. Some starting material and sulphur intermediate could be recovered from the reaction mixture.

LR-MS: Calcd for C₉H₁₃FNO₅: 234.1. Found: 234.0 [M+2H-t-Butyl].

### Example 2

### Elongation with a Typical P2

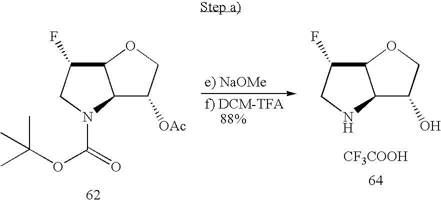

To a solution of compound 62 (0.49 g, 1.7 mmol) in methanol (9.5 ml) was added 0.5 M methanolic sodium methoxide (1 ml), then stirred at rt for 30 min (TLC: Toluene-ethyl acetate 3:2, ninhydrine staining). Methanol washed Dowex W X 8 (50-100 mesh, H⁺-form) was carefully added (pH was monitored by pH-paper) was added until neutral, then the mixture was filtered and concentrated. The residue was dissolved in dichloromethane and trifluoroacetic acid was added at 0° C. The reaction mixture was then stirred at rt for 55 min (TLC: dichloromethane-methanol 9:1, ninhydrine staining), then concentrated. Column chromatography (diameter: 2 cm, silica: 15 g, packing eluent: dichloromethane-methanol 95:5) of the residue (dissolved in dichloromethane-methanol 95:5) using methanol in dichloromethane 5:95 (150 ml), 7:93 (100 ml) and 1:9 (200 ml) gave a hard syrup which crystallized upon standing (0.39 g, 1.50 mmol, 88%).

NMR data (400 MHz, DMSO-d6): ¹H, 3.34, 3.44 (2 dd, 1H, H-6a), 3.60-3.70 (m, 2H, H-1a and H-6b), 3.89 (dd, 1H, H-1b), 4.15 (d, 1H, H-3), 4.51 (brs, 1H, H-2), 4.76 (dd, 1H, H-4), 5.26 (dd, ²J_{H,F}=48.3 Hz, H-5).

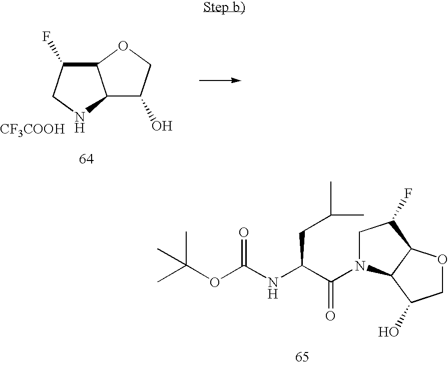

To a stirred solution of compound 64 (0.34 g, 1.30 mmol), N-ethyl-N′-(3-dimethylaminopropyl)carbodiimide hydrochloride (0.28 g, 1.43 mmol), 1-hydroxybenzotriazole hydrate (0.22 g) and N-(tert-Butoxycarbonyl)-L-leucine monohydrate (0.34 g, 1.37 mmol) in DMF (10 ml) was added triethylamine (0.54 ml, 3.9 mmol), then stirred at rt for 24 h. The reaction mixture was the partitioned between 10% aq. citric acid (30 ml) and ethyl acetate (10 ml). The water layer was extracted with ethyl acetate (3×10 ml), then the organic layers were combined, and washed successively with water (1×20 ml) and 1 M aq. sodium hydrogen carbonate (3×20 ml), then dried (sodium sulfate), filtered and concentrated onto silica. Flash chromatography with ethyl acetate in petroleum ether (40-60%, stepwise gradient elution) of the residue gave 15 (0.35 g, 0.98 mmol, 75%) as a colourless amorphous solid.

LR-MS: Calcd for C₁₃H₂₂FN₂O₅: 305.1. Found: 305.1 [M+2H-t-Butyl].

### Example 3

### Oxidation to P1 Ketone

This example shows oxidation of a model P3+P2+hydroxylated P1 compound.

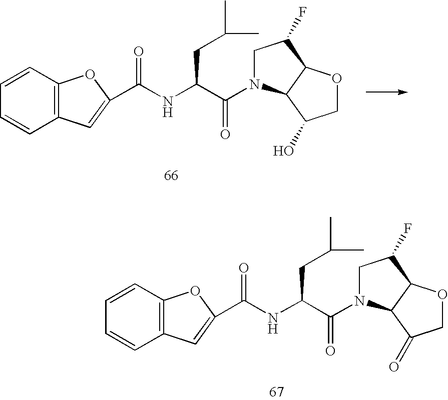

To a stirred solution of compound 66 (0.10 g, 0.25 mmol) in dichloromethane (4 ml) at rt was added Dess-Martin periodinane (0.12 g, 0.28 mmol). After stirring for 90 minutes the reaction mixture was diluted with dichloromethane (10 ml), washed with 1:1 μM aq. sodium hydrogen carbonate-10% aq. sodiumthiosulfate (4×10 ml), then dried (sodium sulfate), filtered and concentrated onto silica. Flash chromatography with ethyl acetate in petroleum ether (50-60%, stepwise gradient elution) of the residue gave 67 (0.072 g, 0.18 mmol, 71%) as a colourless foam. Compound 67 is obtained as a mixture of geometrical isomers (rotamers) and their hydrates.

LR-MS: Calcd for C₂₁H₂₄FN₂O₅: 403.2. Found: 403.0 [M+H]. A NMR sample of the ketoforms of 67 was obtained as follows; 5 mg of compound 67 (mixture of geometrical isomers and hydrate forms with the ratio: hydrate/keto 6:4) was dissolved in DMSO-d₆, then heated up to 100° C. in the NMR apparatus and then allowed to reach 50° C. upon which NMR indicated only trace amounts of the hydrate forms and the ratio of the rotamers were 2:1.

NMR data (500 MHz, DMSO-d6, 50° C.): ¹H, 0.90-1.04 (m, 4×CH₃, major and minor forms), 1.39-1.82 (m, 2×CH₂CH(CH₃)₂ and 2×CH₂CH(CH₃)₂, major and minor forms), 3.56 (m, H-6a, minor), 3.82 (m, H-6A, major), 3.97-4.25 (m, 4×H-1, major and minor forms and H-6b, minor), 4.37 (dd, H-6b, major), 4.62 (d, H-3, minor), 4.79 (m, H, major), 4.84 (d, H-3, major), 4.94 (m, H-4, major), 5.12 (m, H-4, minor), 5.15-5.34 (m, H-5 major and H-5 minor, H minor, J_{H,F major}=49.1 Hz, J_{H,F minor}=49.4 Hz), 7.35 (t, 1H, Ar—H), 7.47 (t, 1H, Ar—H), 7.57-7.70 (m, 2H, Ar—H), 7.78 (d, 1H, Ar—H), 8.18 (d, —NH, minor), 8.70 (d, —NH, major).

### Example 4

### An Alternative P1 Epimer

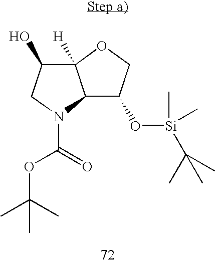

To a stirred solution of compound (60) (1.58 g, 4.19 mmol) in methanol (20 mL) was added a solution of 0.5 M sodium methoxide in methanol (5 mL) at room temperature, then stirred for 40 min. The reaction mixture was then neutralized with Dowex 50 WX 8 (H⁺-form), filtered, added triethylamine until slight alkaline, then concentrated and concentrated from toluene (2×20 mL). To a stirred solution of the residue and imidazole (0.43 g, 6.28 mmol) in DMF (10 mL) at 0° C. was added tert-Butyldimethylchlorosilane (0.76 g, 5.02 mmol), then stirred at room temperature overnight. The reaction mixture was then diluted with ethyl acetate (100 mL), washed successively with 10% aq. citric acid (3×50 mL) and 1M aq. sodium hydrogen carbonate (3×50 mL), dried (sodium sulphate), filtered and concentrated onto silica. Column chromatography (stepwise gradient elution, ethyl acetate in toluene, 5-20%) of the residue afforded the fully protected intermediate as a syrup (1.86 g). A mixture of palladium on charcoal (Aldrich 10%, 0.28 g) and the intermediate obtained above (1.80 g, 4.00 mmol) in ethyl acetate (40 mL) was hydrogenated at slight overpressure for 1 h, then filtered through celite and concentrated.

The material crystallized upon drying in vacuum to afford 72 as needles (1.34 g, 90%).

NMR data (400 MHz, CDCl₃): ¹H, delta 0.14 (m, 6H, Si(CH₃)₂), 0.90 (m, 9H, SiC(CH₃)₃), 1.48 (m, 9H, C(CH₃)₃), 2.53 (m, 1H, OH), 2.78 (dd, 1H, —H-6A), 3.67-4.05 (m, 3H, H-1A, H-1B and H-6B), 4.05-4.21 (m, 2H, H-3 and H-5), 4.35-4.50 (2 brs, 1H, H-2), 4.57 (m, 1H, H-4).

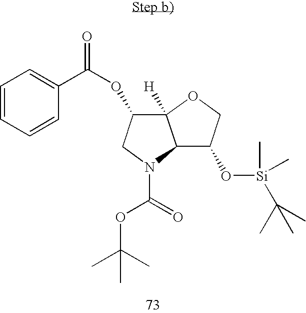

To a stirred solution of (72) (1.068 g, 2.97 mmol), benzoic acid (0.50 g, 4.46 mmol) and triphenylphosphine (1.17 g, 4.46 mmol) in THF (15 mL) at 0° C. was added dropwise a solution of diisopropyl azodicarboxylate (0.88 mL, 4.46 mmol) in THF (5 mL) during 20 minutes. The reaction mixture was then stirred at room temperature overnight, then concentrated onto silica. Flash chromatography of the residue using petroleum ether-ethyl acetate 9:1 as eluent, gave a colorless syrup (1.34 g, 97%).

NMR data (400 MHz, CDCl3): 1H, delta 0.08-0.21 (m, 6H, Si(CH₃)₂), 0.90 (s, 9H, SiC(CH₃)₃), 1.42-1.56 (m, 9H, C(CH₃)₃), 3.48 (m, 1H, H-6A), 3.70-4.01 (m, 3H, H-1A, H-1B, H-6B minor and major), 4.21, 4.30 (2d, 1H, H-3), 4.44, 4.56 (2 brs, 1H, H-2), 4.72 (m, 1H, H-4), 5.34 (d, 1H, H-5), 7.45 (t, 2H, Ar—H), 7.58 (t, 1H, Ar—H), 8.00 (d, 2H, Ar—H).

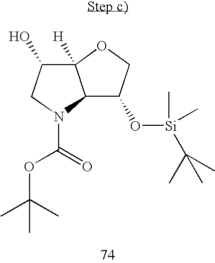

To a stirred solution of (73) (1.34 g, 2.89 mmol) in methanol (6 mL) was added a solution of 0.5 M sodium methoxide in methanol (6 mL) at room temperature, then stirred for 15 min. The reaction mixture was then neutralized with Dowex 50 WX 8 (H⁺-form) and filtered. The obtained solution was added a solution obtained similarly as above starting from (II) (0.187 g, 0.40 mmol), then concentrated. Flash chromatography of the residue using toluene-ethyl acetate 3:2 as eluent gave 74 as a colorless syrup which crystallized upon drying in vacuum (1.091 g, 92%).

NMR data (400 MHz, CDCl3): 1H, delta 0.06-0.20 (m, 6H, Si(CH₃)₂), 0.89 (s, 9H, SiC(CH₃)₃), 1.42-1.54 (m, 9H, C(CH₃)₃), 2.03 (brs, 1H, OH), 3.28 (dd, 1H, H-6A), 3.53-3.79 (m, 3H, H-1A, H-1B, H-6B), 4.19 and 4.34-4.56 (2 m, 4H, H-2, H-3, H-4 and H-5).

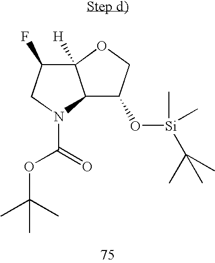

To a stirred solution of (74) (0.428 g, 1.19 mmol) in dichloromethane (10 mL) in a Teflon coated flask was added Deoxofluor (50% in THF, 0.53 mL) at room temperature resulting in a slight temperature increase. The reaction mixture was stirred at room temperature for 72 h, then diluted with dichloromethane (20 mL), washed with 1M aq. sodium hydrogen carbonate (2×20 mL), dried (sodium sulphate), filtered and concentrated onto silica. Flash chromatography of the residue using petroleum ether-ethyl acetate 9:1 as eluent gave (IV) as a colorless oil (0.118 g, 27%).

NMR data (400 MHz, CDCl3): 1H, delta 0.08-0.20 (m, 6H, Si(CH₃)₂), 0.89 (s, 9H, SiC(CH₃)₃), 1.42-1.53 (m, 9H, C(CH₃)₃), 3.26 and 3.36 (2 dd, 1H, H-6A), 3.64 (m, 1H, H-1A), 3.73-4.04 (m, 3H, H-1B, H-6B), 4.20 (dd, 1H, H-3*), 4.40, 4.51 (2 s, 1H, H-2), 4.69 (m, 1H, H-4*) 4.86, 4.98 (2 brs, 1H, H-5). * Could be interchanged.

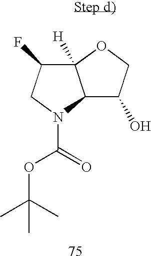

To a stirred solution of (75) (0.229 g, 0.63 mmol) in THF (8 mL) was added 1 M tetrabutylammonium fluoride in THF (0.70 mL), then stirred at room temperature for 40 min. The reaction mixture was then concentrated onto silica. Column chromatography of the residue using toluene-ethyl acetate 1:1 as eluent gave 75 as a colorless hard syrup (0.150 g, 96%).

NMR data (400 MHz, CDCl3): 1H, delta 1.46-1.53 (m, 9H, C(CH₃)₃), 2.70 (d, 0.3H, OH-minor), 3.26-3.46 (m, 1.7H, H-6A and OH-major), 3.75-4.04 (m, 3H, H-1A, H-1B and H-6B), 4.29, 4.34 (2d, 1H, H-3* minor and major), 4.43, 4.50 (2 brs, 1H, H-2 minor and major), 4.74 (m, 1H, H-4*), 4.89, 5.02 (2 brs, 1H, H-5).

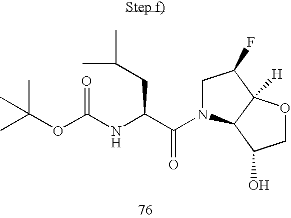

To a solution of (75) (0.099 g, 0.40 mmol) in dichloromethane (2 mL) at 0° C., was added trifluoroacetic acid (2 mL), then stirred at room temperature for 35 min, then concentrated and concentrated from toluene (3×5 mL). To a suspension of the residue, 1-hydroxybenzotrazole hydrate (0.067 g, 0.44 mmol), N-ethyl-N′-(3-dimethylaminopropyl)carbodiimide x HCl (0.084 g, 0.44 mmol) and N-(tert-Butoxycarbonyl)-L-leucine monohydrate (0.105 g, 0.42 mmol) in DMF (4 mL) was added triethylamine (0.17 mL, 1.2 mmol), then stirred at room temperature overnight. The reaction was then concentrated into half the volume, diluted with ethyl acetate (25 mL), washed successively with 10% aq. citric acid (3×15 mL), and 1M aq. sodium hydrogen carbonate (3×15 mL), dried (sodium sulphate), filtered and concentrated. Column chromatography of the residue using ethyl acetate-toluene 3:2 afforded (76) as a colorless hard syrup (0.137 g, 95%).

NMR data (400 MHz, CDCl₃, selected signals): 1H, delta 0.89-1.01 (m, 6H, C(CH)₂), 4.98, 5.07 (2 dd, 1H, H-5major and H-5 minor).

LR-MS: Calcd for C₁₇H₃₀FN₂O₅: 361.2. Found: 361.1 [M+H].

LR-MS: Calcd for C₂₁H₂₆FN₂O₅: 405.2. Found: 405.1 [M+H].

The P2-P1 building block is coupled with a suitable haloalkylylated capping group and the P1 hydroxy group oxidised to the ketone as shown in the model compound above.

### Example 5

### Novel P2 Building Block

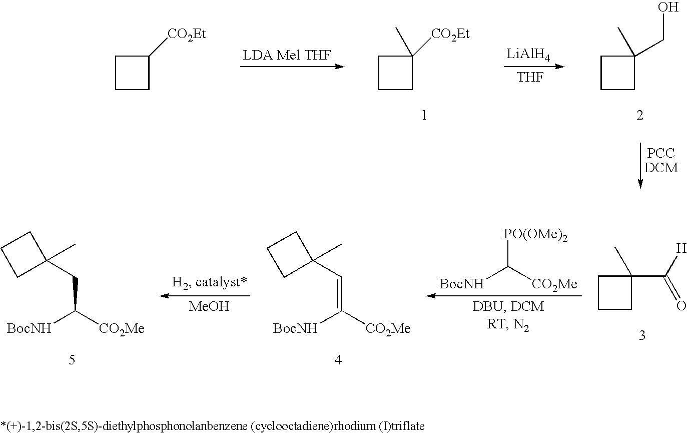

1-Methyl-cyclobutanecarboxylic acid ethyl ester 1 was prepared from ethyl cyclobutanecarboxylate by the method described in J. Am. Chem. Soc., Vol. 103 No. 2 1981 436-442.

1-Methyl-cyclobutanecarboxylic acid ethyl ester 1 (1 eq) was stirred under a nitrogen atmosphere at 0° C. in anhydrous THF. To this solution was added portionwise lithium aluminium hydride (1.5 eq) and the suspension was stirred at room temperature for 3 hours. The reaction mixture was cooled on ice, treated with 1M HCl (aq) and stirred at 0° C. 20 minutes. The solution was passed through a pad of celite and the filtrate extracted into diethyl ether. The organic phases were dried over MgSO₄, filtered and concentrated in vacuo to give (1-methyl-cyclobutyl)-methanol, 2.

Pyridinium chlorochromate (1.25 eq) and the same weight of celite were taken up as a suspension in anhydrous dichloromethane. To this was added dropwise a solution of compound 2 (1 eq) in anhydrous dichloromethane and the resulting heterogeneous mixture was stirred at room temperature for 3 hours. The reaction mixture was passed through a pad of silica, eluting with 19:1 isohexanes:ethyl acetate to give 1-methylcyclobutanecarboxaldehyde, 3.

Compound 3 (1 eq) was dissolved with stirring in anhydrous dichloromethane, and to this was added Boc-phosphoglycine trimethyl ester (0.5 eq) and 1,8-diazabicyclo[5.4.0]undec-7-ene (1.2 eq). The resulting solution was stirred at ambient temperature under nitrogen overnight. The reaction mixture was partitioned between dichloromethane and successively 1M HCl (aq), sat. NaHCO₃ (aq) and sat. NaCl (aq). The organic layer was dried over MgSO₄, filtered and concentrated in vacuo. The resulting oil was purified by flash column chromatography, eluting with 1% methanol in dichloromethane to give 2-tert-butoxycarbonylamino-3-(1-methyl-cyclobutyl)-acrylic acid methyl ester, 4.

Compound 4 was dissolved in anhydrous methanol and degassed with nitrogen. (+)-1,2-bis(2S,5S)-diethylphosphonolanbenzene (cyclooctadiene)rhodium (I) triflate was added and degassing was continued for a further 10 minutes. The reaction was shaken under a hydrogen atmosphere (4 bar) for 48 hours. The solution was concentrated. in vacuo and purified by flash chromatography, eluting with dichloromethane, to give 2S-tert-butoxycarbonylamino-3-(1-methyl-cyclobutyl)-propionic acid methyl ester, 5.

HPLC retention time 5.88 min (monitored at 215 and 254 nm)

HPLC using Synergy Max RP 80 μm 50×4.6 mm column, 10→90% 6 min gradient of solution B (solution A=0.1% TFA in water and solution B=10% A in acetonitrile) at flow rate of 2 ml/min.

MS [M+H]⁺ 272.08 (20%) [M-Boc+H]⁺ 172.06 (100%)

Electrospray ionisation, eluting with acetonitrile/ammonium formate buffer.

¹H NMR (400 MHz, CDCl₃. 4.89-4.79 (1H, m) 4.33-4.27 (1H, m) 3.71 (3H, s) 1.98-1.62 (8H, m) 1.42 (9H, s) 1.22 (3H, s)

### Example 6

### (S)-2-[(S)-1-(4-Bromo-phenyl)-2,2,2-trifluoro-ethylamino]-4-methyl-pentanoic acid

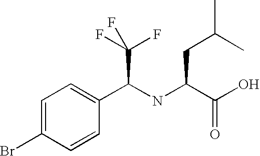

The title compound is prepared as shown in Li, C. S. et al Bioorg. Med. Chem. Lett. 2006, 16, 1985

### Example 7

### (S)-4-Isobutyl-2-trifluoromethyl-oxazolidine

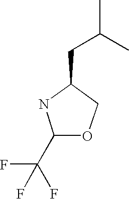

The title compound is prepared as shown in Ishii, A. et al Synlett 1997, 1381.

### Example 8

### [(S)-1-(tert-Butyl-dimethyl-silanyloxymethyl)-3-methyl-butyl]-[2,2,2-trifluoro-eth-(E)-ylidene]-amine

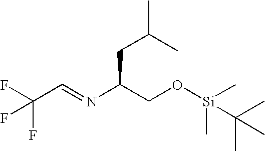

The title compound is prepared as shown in Li, C. S. et al Bioorg. Med. Chem. Lett. 2006, 16, 1985

### Example 9

### (3R,3aR,6S,6aS)-6-Fluoro-3-hydroxy-hexahydro-furo[3,2-b]pyrrol-4-ium; chloride

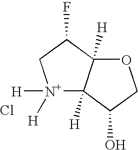

The title compound is prepared by deprotection of the building block of Example 1 and treatment with hydrochloric acid.

### Example 10

### (S)-2-[1-(4-Bromo-phenyl)-2,2,2-trifluoro-ethylamino]-4-methyl-pentan-1-ol

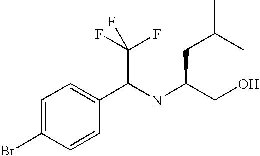

To a stirred solution of 1,4 dibromobenzene (1.79 g, 7.61 mmol) in dry THF (5 mls) under N2 at −78° C. was added dropwise 1.6M butyl lithium in hexanes (4.5 mls, 7.61 mmol). The resulting solution was stirred for a further 20 minutes at −78° C. after which a solution of (S)-4-isobutyl-2-trifluoromethyl-oxazolidine (0.5 g, 2.54 mmol) in dry THF (5 mls) was added dropwise to the solution of the aryl lithium. Stirring was continued for a further 1 hour at −78° C. The reaction mixture was quenched with 5 mls of 2M hydrochloric acid solution and the mixture allowed to warm to room temperature. The solution was basified with 10 mls of sodium hydroxide and the resulting solution was extracted with Ethyl Acetate (2×30 mls) and the combined organic fractions were dried (MgSO₄) and concentrated in vacuo. The product was purified by flash column chromatography (ethyl acetate:iso-hexane 1:4) to yield the title product as a yellow oil (0.620 g, 53%) which is a 2:1 mixture of diasterisomers. MS M+H 355, Retention Time 4.9 & 5.1 mins 10-90 MeCN:0.05% TFA 6 min Gradient C12 Reverse Phase 50 mm*4.6 mm i.d. column

### Example 11

### (S)-4-Methyl-2-[2,2,2-trifluoro-1-(4′-methanesulfonyl-biphenyl-4-yl)-ethylamino]-pentan-1-ol

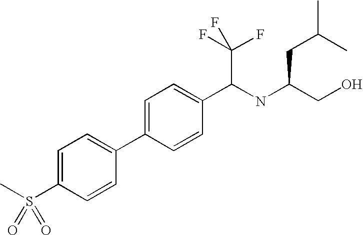

To a stirred solution of (S)-2-[1-(4-bromo-phenyl)-2,2,2-trifluoro-ethylamino]-4-methyl-pentan-1-ol (0.1 g, 0.28 mmol) in DMF (5 mls) was added (4-methane sulphonyl phenyl) boronic Acid (0.067 g, 0.34 mmol), sodium carbonate solution (0.090 g, 0.85 mmol in 5 mls of water) and [1,1′-bis(diphenylphosphino)ferrocene]palladium(II) chloride 1:1 complex with CH₂Cl₂ (0.023 g, 0.03 mmol). The resulting solution was warmed to 80° C. for 60 minutes. The reaction mixture was allowed to cool to room temperature and diluted with CH₂Cl₂ (20 mls). The organics were separated, dried (MgSO₄)) and concentrated in vacuo. The product was purified by flash column chromatography (iso-hexane:Ethyl Acetate, 5-66% Gradient) to yield the title product as a yellow solid (0.087 g, 53%) which is a 2:1 mixture of diasterisomers. Retention Time 4.2 & 4.3 mins 10-90 MeCN:0.05% TFA 6 min Gradient C12 Reverse Phase 50 mm*4.6 mm i.d. column

### Example 12

### (S)-4-Methyl-2-[(S)-2,2,2-trifluoro-1-(4′-methanesulfonyl-biphenyl-4-yl)-ethylamino]-pentanoic acid & (S)-4-Methyl-2-[(R)-2,2,2-trifluoro-1-(4′-methanesulfonyl-biphenyl-4-yl)-ethylamino]-pentanoic acid

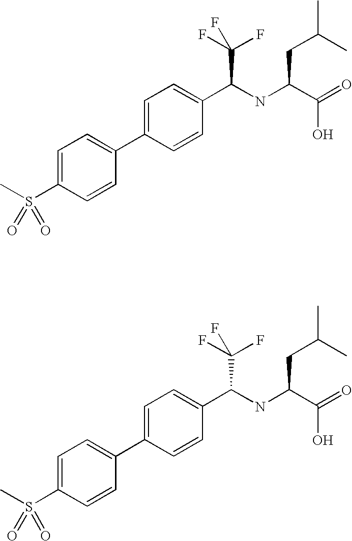

To a stirred suspension of periodic acid (5.7 g, 4.19 mmol) in acetonitrile (55 mls) was added chromium(VI) oxide (11 mgs, 0.011 mmol) and water (0.25 mls). The resulting suspension was stirred several hours at room temperature and then cooled to 0° C. on an ice bath and (S)-4-methyl-2-[2,2,2-trifluoro-1-(4′-methanesulfonyl-biphenyl-4-yl)-ethylamino]-pentan-1-ol (1.8 g, 4.19 mmol) was added dropwise in 10 mls of acetonitrile, stirring was continued at 0° C. for a further 30 minutes. The reaction was poured into acidified Na₂HPO₄ solution (12.0 g in 200 mls water adjusted to pH 3 with conc. HCl) and then extracted with diethyl ether (3×100 mls), the combined organic extracts were washed consecutively with Brine (1×100 mls), sodium hydrogen sulphite solution (1×100 mls) and brine (1×100 mlls). The organic extract was dried (Na₂SO₄) and concentrated to give the as title product as a yellow oil (1.3 g, 53%) which is a 2:1 mixture of diasterisomers. Retention Time 4.2 & 4.4 mins 10-90 MeCN:0.05% TFA 6 min Gradient C12 Reverse Phase 50 mm*4.6 mm i.d. column. The diastereomeric mixture of products was further purified by prep-HPLC [Phenomenex Synergi C₁₂ 10 μm 10×150 mm column, 30→90% 15 min gradient of solution B (solution A=0.1% TFA in water and solution B=10% A in acetonitrile) at flow rate of 18 ml/min] to yield the two separate diasteroisomers of the title compound as white solids in a 2:1 ratio, (S)-4-Methyl-2-[(S)-2,2,2-trifluoro-1-(4′-methanesulfonyl-biphenyl-4-yl)-ethylamino]-pentanoic acid (0.192 g) M+H 444, (S)-4-Methyl-2-[(R)-2,2,2-trifluoro-1-(4′-methanesulfonyl-biphenyl-4-yl)-ethylamino]-pentanoic acid (0.120 g) M+H 444.

### Example 13

### (S)-1-((3R,3aR,6S,6aS)-6-Fluoro-3-hydroxy-hexahydro-furo[3,2-b]pyrrol-4-yl)-4-methyl-2-[(R)-2,2,2-trifluoro-1-(4′-methanesulfonyl-biphenyl-4-yl)-ethylamino]-pentan-1-one

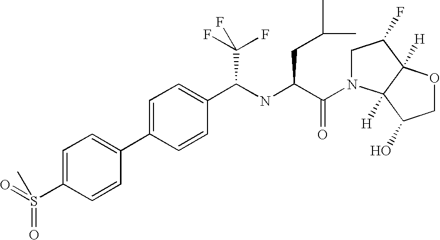

To a stirred solution of (3R,3aR,6S,6aS)-6-fluoro-3-hydroxy-hexahydro-furo[3,2-b]pyrrol-4-ium; chloride (0.05 g, 0.30 mmol) in CH₂Cl₂ (2 mls) and (S)-4-methyl-2-[(R)-2,2,2-trifluoro-1-(4′-methanesulfonyl-biphenyl-4-yl)-ethylamino]-pentanoic acid (0.120 g, 0.27 mmol) was added dicyclohexyl carbodiimide (0.12 g, 0.54 mmol) and diisopropyl ethyl amine (0.047 mls, 0.27 mmol). The resulting solution was stirred at room temperature for 60 minutes. The reaction was then filtered through Celite and then the organics washed with 2M HCl (1×2 mls), saturated NaHCO₃ (1×2 mls), dried (MgSO₄) and concentrated in vacuo. The product was purified by flash column chromatography (iso-hexane:Ethyl Acetate, 25-100% gradient) to yield the title product as a white foam (0.061 g, 39%), M+H 573, Retention Time 5.8 mins 10-90 MeCN:0.05% TFA 6 min Gradient C12 Reverse Phase 50 mm*4.6 mm i.d. column.

### Example 13A

### (S)-1-((3R,3aR,6S,6aS)-6-Fluoro-3-hydroxy-hexahydro-furo[3,2-b]pyrrol-4-yl)-4-methyl-2-[(S)-2,2,2-trifluoro-1-(4′-methanesulfonyl-biphenyl-4-yl)-ethylamino]-pentan-1-one

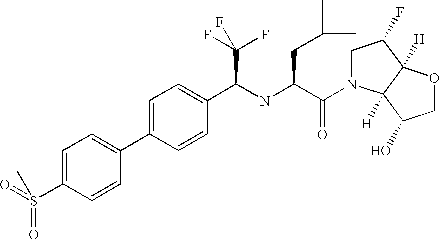

To a stirred solution of (3R,3aR,6S,6aS)-6-fluoro-3-hydroxy-hexahydro-furo[3,2-b]pyrrol-4-ium; chloride (0.08 g, 0.47 mmol) in CH₂Cl₂ (2 mls) and (S)-4-methyl-2-[(S)-2,2,2-trifluoro-1-(4′-methanesulfonyl-biphenyl-4-yl)-ethylamino]-pentanoic acid (0.19 g, 0.43 mmol) was added dicyclohexyl carbodiimide (0.18 g, 0.86 mmol) and diisopropyl ethyl amine (0.074 mls, 0.43 mmol). The resulting solution was stirred at room temperature for 60 minutes. The reaction was then filtered through Celite and then the organics washed with 2M HCl (1×2 mls), saturated NaHCO₃ (1×2 mls), dried (MgSO₄) and concentrated in vacuo. The product was purified by flash column chromatography (iso-hexane:Ethyl Acetate, 25-100% gradient) to yield the title product as a white foam (0.072 g, 29%), M+H 573, Retention Time 5.8 mins 10-90 MeCN:0.05% TFA 6 min Gradient C12 Reverse Phase 50 mm*4.6 mm i.d. column.

### Example 14

### (3aS,6S,6aS)-6-Fluoro-4-[(S)-4-methyl-2-{(S)-2,2,2-trifluoro-1-(4′-methanesulfonyl-biphenyl-4-yl)-ethylamino]-pentanoyl}-tetrahydro-furo[3,2-b]pyrrol-3-one

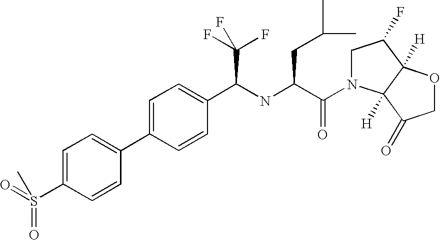

To a stirred solution of (S)-1-((3R,3aR,6S,6aS)-6-fluoro-3-hydroxy-hexahydro-furo[3,2-b]pyrrol-4-yl)-4-methyl-2-[(S)-2,2,2-trifluoro-1-(4′-methanesulfonyl-biphenyl-4-yl)-ethylamino]-pentan-1-one (0.072 g, 0.13 mmol) in CH₂Cl₂ (5 mls) was added Dess Martin periodinane (0.11 g, 0.25 mmol). The resulting solution was stirred at room temperature for 2 hours. The reaction mixture was diluted with CH₂Cl₂ (20 mls) washed with saturated NaHCO₃ (2×10 mls), dried (MgSO₄) and concentrated in vacuo. The product was purified by prep-HPLC [Phenomenex Synergi C₁₂ 10 μm 10×150 mm column, 30→90% 15 min gradient of solution B (solution A=0.1% TFA in water and solution B=10% A in acetonitrile) at flow rate of 18 ml/min] to yield the of the title compound as a white solid (0.038 g, 52%) as a mixture with its ketone hydrate. MS M+H 571, M+H₂O+H 589. Retention Time 5.5 & 5.9 mins 10-90 MeCN:0.05% TFA 6 min Gradient C12 Reverse Phase 50 mm*4.6 mm i.d. column

### Example 15

### (3aS,6S,6aS)-6-Fluoro-4-{(S)-4-methyl-2-[(R)-2,2,2-trifluoro-1-(4′-methanesulfonyl-biphenyl-4-yl)-ethylamino]-pentanoyl}-tetrahydro-furo[3,2-b]pyrrol-3-one

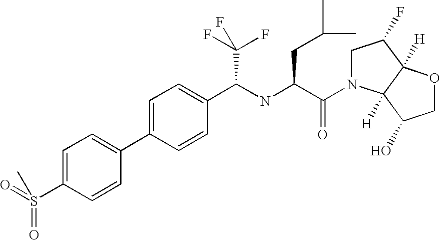

The technique described in Example 14 was applied to (S)-1-((3R,3aR,6S,6aS)-6-fluoro-3-hydroxy-hexahydro-furo[3,2-b]pyrrol-4-yl)-4-methyl-2-[(R)-2,2,2-trifluoro-1-(4′-methanesulfonyl-biphenyl-4-yl)-ethylamino]-pentan-1-one (0.061 g, 0.11 mmol). The product was purified by prep-HPLC [Phenomenex Synergi C₁₂ 10 μm 10×150 mm column, 30→90% 15 min gradient of solution B (solution A=0.1% TFA in water and solution B=10% A in acetonitrile) at flow rate of 18 ml/min] to obtain the title compound (0.021 g, 34%) as a mixture with its ketone hydrate. MS M+H 571, M+H₂O+H 589. Retention Time 5.3 & 5.8 mins 10-90 MeCN:0.05% TFA, 6 min Gradient C12 Reverse Phase 50 mm*4.6 mm i.d. column.

### Example 16

### (S)-2-[(S)-1-(4-Bromo-phenyl)-2,2,2-trifluoro-ethylamino]-1-((3R,3aR,6S,6aS)-6-fluoro-3-hydroxy-hexahydro-furo[3,2-b]pyrrol-4-yl)-4-methyl-pentan-1-one

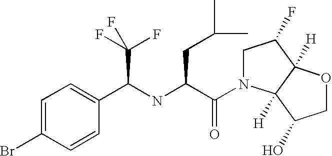

To a stirred solution of (3R,3aR,6S,6aS)-6-fluoro-3-hydroxy-hexahydro-furo[3,2-b]pyrrol-4-ium; chloride (2.250 g, 12.25 mmol) in DMF (40 mls) and (S)-2-[(S)-1-(4-bromo-phenyl)-2,2,2-trifluoro-ethylamino]-4-methyl-pentanoic acid (4.1 g, 11.14 mmol) was added HATU (5.08 g, 13.36 mmol) and diisopropyl ethyl amine (5.8 mls, 33.41 mmol). The resulting solution was stirred at room temperature overnight then concentrated in vacuo. The residue was then dispersed in water (50 mls) and extracted with ethyl acetate (2×150 mls). The combined organic fractions were washed with saturated sodium bicarbonate solution (1×100 mls), and dried over magnesium sulphate and concentrated. The product was purified by flash column chromatography (iso-hexane:ethyl acetate, 5-66% gradient) to yield the title product as a yellow oil (2.95 g, 53%) MS M+H 497, Retention Time 5.8 mins 10-90 MeCN:0.05% TFA 6 min Gradient C12 Reverse Phase 50 mm*4.6 mm i.d. column.

### Example 17

### (S)-1-((3R,3aR,6S,6aS)-6-Fluoro-3-hydroxy-hexahydro-furo[3,2-b]pyrrol-4-yl)-4-methyl-2-[(S)-2,2,2-trifluoro-1-(3′-methanesulfonyl-biphenyl-4-yl)-ethylamino]-pentan-1-one

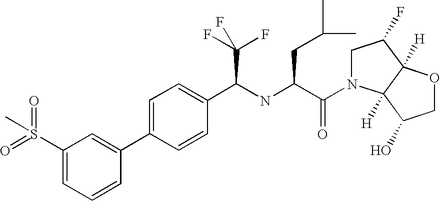

To a stirred solution of (S)-2-[(S)-1-(4-bromo-phenyl)-2,2,2-trifluoro-ethylamino]-1-((3R,3aR,6S,6aS)-6-fluoro-3-hydroxy-hexahydro-furo[3,2-b]pyrrol-4-yl)-4-methyl-pentan-1-one (0.1 g, 0.20 mmol) in DMF (1 ml) was added (3-methanesulfonyl phenyl)boronic acid (0.044 g, 0.22 mmol), 2M sodium carbonate solution (1 ml) and [1,1′-bis(diphenylphosphino)ferrocene]palladium(II) chloride 1:1 complex with CH₂Cl₂ (0.016 g, 0.02 mmol). The resulting solution was sealed in a tube and heated in a microwave to 160° C. for 5 minutes. The reaction mixture was allowed to cool to room temperature and diluted with CH₂Cl₂:H₂O (1:1, 10 mls). The organics were separated, dried (MgSO₄)) and concentrated in vacuo. The product was purified by flash column chromatography (iso-hexane:ethyl acetate, 5-66% gradient) to yield the title product as a yellow oil (0.048 g, 42%). MS M+H 573. Retention Time 5.4 mins 10-90 MeCN:0.05% TFA 6 min Gradient C12 Reverse Phase 50 mm*4.6 mm i.d. column.

### Example 18

### (3aS,6S,6aS)-6-Fluoro-4-{(S)-4-methyl-2-[(S)-2,2,2-trifluoro-1-(3′-methanesulfonyl-biphenyl-4-yl)-ethylamino]-pentanoyl}-tetrahydro-furo[3,2-b]pyrrol-3-one

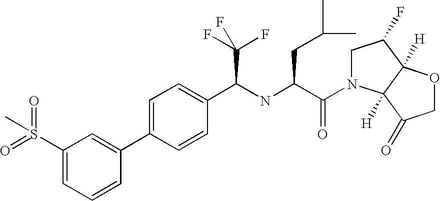

The technique described in Example 14 was applied to (S)-1-((3R,3aR,6S,6aS)-6-fluoro-3-hydroxy-hexahydro-furo[3,2-b]pyrrol-4-yl)-4-methyl-2-[(S)-2,2,2-trifluoro-1-(3′-methanesulfonyl-biphenyl-4-yl)-ethylamino]-pentan-1-one (0.048 g, 0.11 mmol). The product was purified by flash column chromatography (iso-hexane:ethyl acetate, 5-66% gradient) to give the title compound (0.013 g, 29%) as a mixture with its ketone hydrate. MS M+H 571, M+H₂O+H 589. Retention Time 4.2 & 4.7 mins 10-90 MeCN:0.05% TFA, 6 min Gradient C12 Reverse Phase 50 mm*4.6 mm i.d. column.

### Example 19

### (S)-1-((3R,3aR,6S,6aS)-6-Fluoro-3-hydroxy-hexahydro-furo[3,2-b]pyrrol-4-yl)-4-methyl-2-[(S)-2,2,2-trifluoro-1-(4′-fluoro-biphenyl-4-yl)-ethylamino]-pentan-1-one

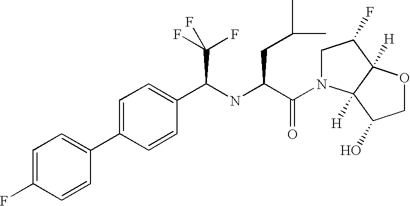

The technique described in Example 17 was applied to (S)-2-[(S)-1-(4-bromo-phenyl)-2,2,2-trifluoro-ethylamino]-1-((3R,3aR,6S,6aS)-6-fluoro-3-hydroxy-hexahydro-furo[3,2-b]pyrrol-4-yl)-4-methyl-pentan-1-one (0.15 g, 0.30 mmol) and 4-fluorophenyl boronic acid. The product was purified by flash column chromatography (iso-hexane:ethyl acetate, 5-66% gradient) to yield the title product as an oil (0.069 g, 45%). MS M+H 513. Retention Time 5.6 mins 30-90 MeCN:0.05% TFA 6 min Gradient C12 Reverse Phase 50 mm*4.6 mm i.d. column.

### Example 20

### (3aS,6S,6aS)-6-Fluoro-4-{(S)-4-methyl-2-[(S)-2,2,2-trifluoro-1-(4′-fluoro-biphenyl-4-yl)-ethylamino]-pentanoyl}-tetrahydro-furo[3,2-b]pyrrol-3-one

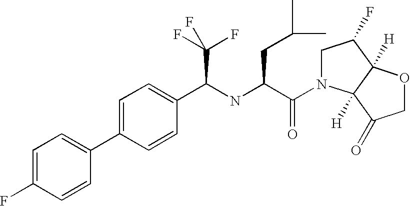

The technique described in Example 14 was applied to (S)-1-((3R,3aR,6S,6aS)-6-fluoro-3-hydroxy-hexahydro-furo[3,2-b]pyrrol-4-yl)-4-methyl-2-[(S)-2,2,2-trifluoro-1-(4′-fluoro-biphenyl-4-yl)-ethylamino]-pentan-1-one (0.069 g, 0.13 mmol). The product was purified by prep-HPLC [Phenomenex Synergi C₁₂ 10 μm 10×150 mm column, 30→90% 15 min gradient of solution B (solution A=0.1% TFA in water and solution B=10% A in acetonitrile) at flow rate of 18 ml/min] to obtain the title compound, white solid (0.036 g, 34%) as a mixture with its ketone hydrate. MS M+H 511, M+H₂O+H 529. Retention Time 5.5 & 6.0 mins 30-90 MeCN:0.05% TFA, 6 min Gradient C12 Reverse Phase 50 mm*4.6 mm i.d. column.

### Example 21

### 4′-{(S)-2,2,2-Trifluoro-1-[(S)-1-((3R,3aR,6S 6aS)-6-fluoro-3-hydroxy-hexahydro-furo[3,2-b]pyrrole-4-carbonyl)-3-methyl-butylamino]-ethyl}-biphenyl-4-carbonitrile

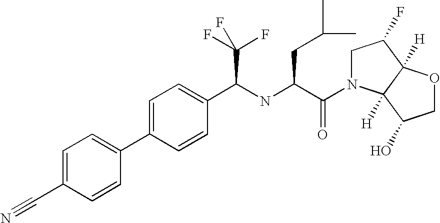

The technique described in Example 17 was applied to (S)-2-[(S)-1-(4-bromo-phenyl)-2,2,2-trifluoro-ethylamino]-1-((3R,3aR,6S,6aS)-6-fluoro-3-hydroxy-hexahydro-furo[3,2-b]pyrrol-4-yl)-4-methyl-pentan-1-one (0.15 g, 0.30 mmol) and 4-cyano phenyl boronic acid. The product was purified by flash column chromatography (iso-hexane:ethyl acetate, 5-66% gradient) to yield the title product as an oil (0.054 g, 35%). MS M+H 513. Retention Time 5.2 mins 30-90 MeCN:0.05% TFA 6 min Gradient C12 Reverse Phase 50 mm*4.6 mm i.d. column.

### Example 22

### 4′-{(S)-2,2,2-Trifluoro-1-[(S)-1-((3aS,6S,6aS)-6-fluoro-3-oxo-hexahydro-furo[3,2-b]pyrrole-4-carbonyl)-3-methyl-butylamino]-ethyl}-biphenyl-4-carbonitrile

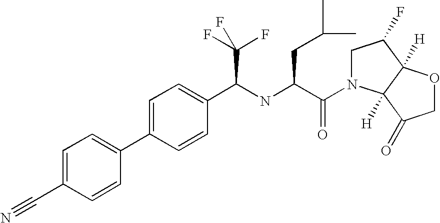

The technique described in Example 14 was applied to 4′-{(S)-2,2,2-trifluoro-1-[(S)-1-((3aS,6S,6aS)-6-fluoro-3-oxo-hexahydro-furo[3,2-b]pyrrole-4-carbonyl)-3-methyl-butylamino]-ethyl}-biphenyl-4-carbonitrile (0.054 g, 0.13 mmol). The product was purified by prep-HPLC [Phenomenex Synergi C₁₂ 10 μm 10×150 mm column, 30→90% 15 min gradient of solution B (solution A=0.1% TFA in water and solution B=10% A in acetonitrile) at flow rate of 18 ml/min] to obtain the title compound, white solid (0.026 g, 34%) as a mixture with its ketone hydrate. MS M+H 518, M+H₂O+H 536. Retention Time 5.1 & 5.6 mins 30-90 MeCN:0.05% TFA, 6 min Gradient C12 Reverse Phase 50 mm*4.6 mm i.d. column.

### Example 23

### (S)-1-((3R,3aR,6S,6aS)-6-Fluoro-3-hydroxy-hexahydro-furo[3,2-b]pyrrol-4-yl)-4-methyl-2-[(S)-2,2,2-trifluoro-1-(4′-trifluoromethyl-biphenyl-4-yl)-ethylamino]-pentan-1-one

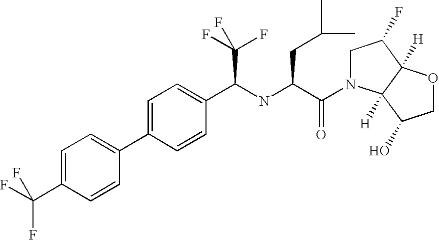

The technique of Example 17 was applied to (S)-2-[(S)-1-(4-bromo-phenyl)-2,2,2-trifluoro-ethylamino]-1-((3R,3aR,6S,6aS)-6-fluoro-3-hydroxy-hexahydro-furo[3,2-b]pyrrol-4-yl)-4-methyl-pentan-1-one (0.15 g, 0.30 mmol) and 4-trifluorophenyl boronic acid. The product was purified by flash column chromatography (iso-hexane:ethyl acetate, 5-66% gradient) to yield the title product as an oil (0.086 g, 51%). MS M+H 563. Retention Time 5.9 mins 30-90 MeCN:0.05% TFA 6 min Gradient C12 Reverse Phase 50 mm*4.6 mm i.d. column.

### Example 24

### (3aS,6S,6aS)-6-Fluoro-4-{(S)-4-methyl-2-[(S)-2,2,2-trifluoro-1-(4′-trifluoromethyl-biphenyl-4-yl)-ethylamino]-pentanoyl}-tetrahydro-furo[3,2-b]pyrrol-3-one

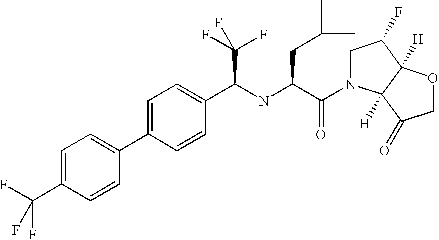

The technique described in Example 14 was applied to (S)-1-((3R,3aR,6S,6aS)-6-fluoro-3-hydroxy-hexahydro-furo[3,2-b]pyrrol-4-yl)-4-methyl-2-[(S)-2,2,2-trifluoro-1-(4′-trifluoromethyl-biphenyl-4-yl)-ethylamino]-pentan-1-one (0.086 g, 0.15 mmol). The product was purified by prep-HPLC [Phenomenex Synergi C₁₂ 10 μm 10×150 mm column, 30→90% 15 min gradient of solution B (solution A=0.1% TFA in water and solution B=10% A in acetonitrile) at flow rate of 18 ml/min] to obtain the title compound, white solid (0.056 g, 66%) as a mixture with its ketone hydrate. MS M+H 561, M+H₂O+H 579. Retention Time 6.0 & 6.5 mins 30-90 MeCN:0.05% TFA, 6 min Gradient C12 Reverse Phase 50 mm*4.6 mm i.d. column.

### Example 25

### (S)-1-((3R,3aR,6S,6aS)-6-Fluoro-3-hydroxy-hexahydro-furo[3,2-b]pyrrol-4-yl)-4-methyl-2-[(S)-2,2,2-trifluoro-1-(2′-fluoro-biphenyl-4-yl)-ethylamino]-pentan-1-one

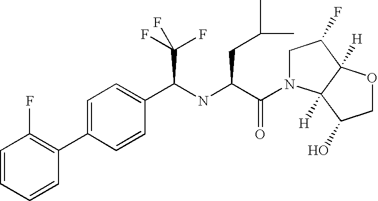

The technique described in Example 17 was applied to (S)-2-[(S)-1-(4-bromo-phenyl)-2,2,2-trifluoro-ethylamino]-1-((3R,3aR,6S,6aS)-6-fluoro-3-hydroxy-hexahydro-furo[3,2-b]pyrrol-4-yl)-4-methyl-pentan-1-one (0.13 g, 0.26 mmol) in dimethyl ether:ethanol (1:1, 1 ml) and 2-fluorophenyl boronic acid. The product was purified by flash column chromatography (iso-hexane:ethyl acetate, 5-66% gradient) to yield the title product as an oil (0.05 g, 38%). MS M+H 513. Retention Time 5.4 mins 30-90 MeCN:0.05% TFA 6 min Gradient C12 Reverse Phase 50 mm*4.6 mm i.d. column.

### Example 26

### (3aS,6S,6aS)-6-Fluoro-4-{(S)-4-methyl-2-[(S)-2,2,2-trifluoro-1-(2′-fluoro-biphenyl-4-yl)-ethylamino]-pentanoyl}-tetrahydro-furo[3,2-b]pyrrol-3-one

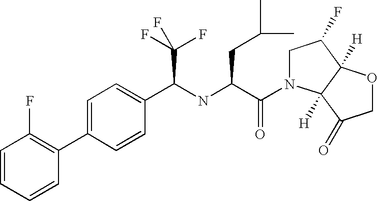

The technique described in Example 14 was applied to (S)-1-((3R,3aR,6S,6aS)-6-fluoro-3-hydroxy-hexahydro-furo[3,2-b]pyrrol-4-yl)-4-methyl-2-[(S)-2,2,2-trifluoro-1-(2′-fluoro-biphenyl-4-yl)-ethylamino]-pentan-1-one (0.05 g, 0.10 mmol). The product was purified by prep-HPLC [Phenomenex Synergi C₁₂ 10 μm 10×150 mm column, 30→90% 15 min gradient of solution B (solution A=0.1% TFA in water and solution B=10% A in acetonitrile) at flow rate of 18 ml/min] to obtain the title compound, white solid (0.036 g, 72%) as a mixture with its ketone hydrate. MS M+H 511, M+H₂O+H 529. Retention Time 5.5 & 6.0 mins 30-90 MeCN:0.05% TFA, 6 min Gradient C12 Reverse Phase 50 mm*4.6 mm i.d. column.

### Example 27

### (S)-1-((3R,3aR,6S,6aS)-6-Fluoro-3-hydroxy-hexahydro-furo[3,2-b]pyrrol-4-yl)-4-methyl-2-[(S)-2,2,2-trifluoro-1-(4′-methylsulfanyl-biphenyl-4-yl)-ethylamino]-pentan-1-one

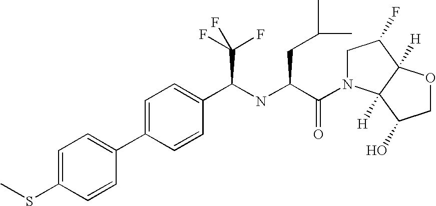

The technique described in Example 17 was applied to (S)-2-[(S)-1-(4-bromo-phenyl)-2,2,2-trifluoro-ethylamino]-1-((3R,3aR,6S,6aS)-6-fluoro-3-hydroxy-hexahydro-furo[3,2-b]pyrrol-4-yl)-4-methyl-pentan-1-one (0.13 g, 0.26 mmol) in dimethyl ether:ethanol (1:1, 1 ml) and 4 methylthiophenyl boronic acid. The product was purified by flash column chromatography (iso-hexane:ethyl acetate, 5-66% gradient) to yield the title product as an oil (0.07 g, 50%). MS M+H 541. Retention Time 5.8 mins 30-90 MeCN:0.05% TFA 6 min Gradient C12 Reverse Phase 50 mm*4.6 mm i.d. column.

### Example 28

### (3aS,6S,6aS)-6-Fluoro-4-{(S)-4-methyl-2-[(S)-2,2,2-trifluoro-1-(4′-methylsulfanyl-biphenyl-4-yl)-ethylamino]-pentanoyl}-tetrahydro-furo[3,2-b]pyrrol-3-one

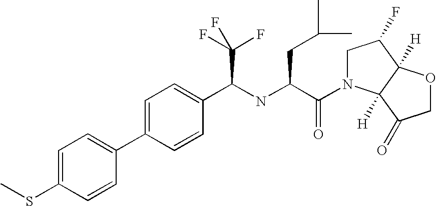

The technique described in Example 14 was applied to (S)-1-((3R,3aR,6S,6aS)-6-fluoro-3-hydroxy-hexahydro-furo[3,2-b]pyrrol-4-yl)-4-methyl-2-[(S)-2,2,2-trifluoro-1-(4′-methylsulfanyl-biphenyl-4-yl)-ethylamino]-pentan-1-one (0.07 g, 0.13 mmol). The product was purified by prep-HPLC [Phenomenex Synergi C₁₂ 10 μm 10×150 mm column, 30→90% 15 min gradient of solution B (solution A=0.1% TFA in water and solution B=10% A in acetonitrile) at flow rate of 18 ml/min] to obtain the title compound, white solid (0.007 g, 10%) as a mixture with its ketone hydrate. MS M+H 539, M+H₂O+H 557. Retention Time 5.7 & 6.3 mins 30-90 MeCN:0.05% TFA, 6 min Gradient C12 Reverse Phase 50 mm*4.6 mm i.d. column.

### Example 29

### (3aS,6S,6aS)-6-Fluoro-4-{(S)-4-methyl-2-[(S)-2,2,2-trifluoro-1-(4′-methylsulfoxide-biphenyl-4-yl)-ethylamino]-pentanoyl}-tetrahydro-furo[3,2-b]pyrrol-3-one

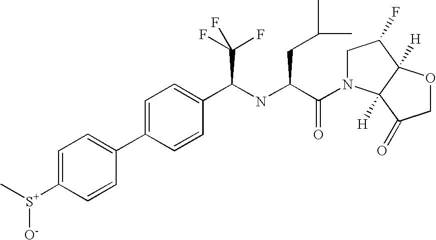

The technique described in Example 14 was applied to (S)-1-((3R,3aR,6S,6aS)-6-fluoro-3-hydroxy-hexahydro-furo[3,2-b]pyrrol-4-yl)-4-methyl-2-[(S)-2,2,2-trifluoro-1-(4′-methylsulfanyl-biphenyl-4-yl)-ethylamino]-pentan-1-one (0.07 g, 0.13 mmol). The product was purified by prep-HPLC [Phenomenex Synergi C₁₂ 10 μm 10×150 mm column, 30→90% 15 min gradient of solution B (solution A=0.1% TFA in water and solution B=10% A in acetonitrile) at flow rate of 18 ml/min] to obtain the title compound, white solid (0.027 g, 39%) as a mixture with its ketone hydrate. MS M+H 555, M+H₂O+H 573. Retention Time 3.9 & 4.5 mins 30-90 MeCN:0.05% TFA, 6 min Gradient C12 Reverse Phase 50 mm*4.6 mm i.d. column.

### Example 30

### (S)-1-((3R,3aR,6S,6aS)-6-Fluoro-3-hydroxy-hexahydro-furo[3,2-b]pyrrol-4-yl)-4-methyl-2-[(S)-2,2,2-trifluoro-1-(4′-methoxy-biphenyl-4-yl)-ethylamino]-pentan-1-one

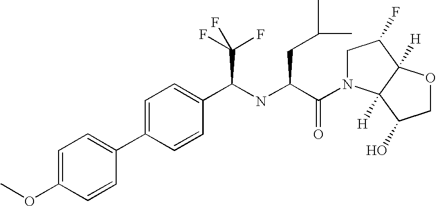

The technique described in Example 17 was applied to (S)-2-[(S)-1-(4-bromo-phenyl)-2,2,2-trifluoro-ethylamino]-1-((3R,3aR,6S,6aS)-6-fluoro-3-hydroxy-hexahydro-furo[3,2-b]pyrrol-4-yl)-4-methyl-pentan-1-one (0.13 g, 0.26 mmol) in dimethyl ether:ethanol (1:1, 1 ml) and 4 methoxyphenyl boronic acid. The product was purified by flash column chromatography (iso-hexane:ethyl acetate, 5-66% gradient) to yield the title product as an oil (0.049 g, 36%). MS M+H 525. Retention Time 5.6 mins 30-90 MeCN:0.05% TFA 6 min Gradient C12 Reverse Phase 50 mm*4.6 mm i.d. column.

### Example 31

### (3aS,6S,6aS)-6-Fluoro-4-{(S)-4-methyl-2-[(S)-2,2,2-trifluoro-1-(4′-methoxy-biphenyl-4-yl)-ethylamino]-pentanoyl}-tetrahydro-furo[3,2-b]pyrrol-3-one

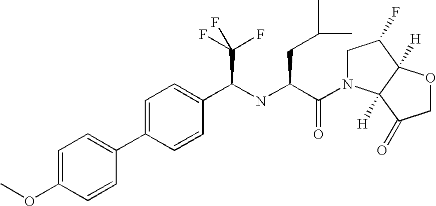

The technique described in Example 14 was applied to (S)-1-((3R,3aR,6S,6aS)-6-fluoro-3-hydroxy-hexahydro-furo[3,2-b]pyrrol-4-yl)-4-methyl-2-[(S)-2,2,2-trifluoro-1-(4′-methoxy-biphenyl-4-yl)-ethylamino]-pentan-1-one (0.049 g, 0.09 mmol). The product was purified by prep-HPLC [Phenomenex Synergi C₁₂ 10 μm 10×150 mm column, 30→90% 15 min gradient of solution B (solution A=0.1% TFA in water and solution B=10% A in acetonitrile) at flow rate of 18 ml/min] to obtain the title compound, white solid (0.033 g, 68%) as a mixture with its ketone hydrate. MS M+H 523, M+H₂O+H 541. Retention Time 5.3 & 5.9 mins 30-90 MeCN:0.05% TFA, 6 min Gradient C12 Reverse Phase 50 mm*4.6 mm i.d. column.

### Example 32

### (S)-1-((3R,3aR,6S,6aS)-6-Fluoro-3-hydroxy-hexahydro-furo[3,2-b]pyrrol-4-yl)-4-methyl-2-[(S)-2,2,2-trifluoro-1-(4′-methyl-biphenyl-4-yl)-ethylamino]-pentan-1-one

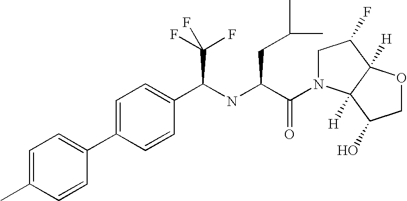

The technique described in Example 17 was applied to (S)-2-[(S)-1-(4-bromo-phenyl)-2,2,2-trifluoro-ethylamino]-1-((3R,3aR,6S,6aS)-6-fluoro-3-hydroxy-hexahydro-furo[3,2-b]pyrrol-4-yl)-4-methyl-pentan-1-one (0.13 g, 0.26 mmol) in dimethyl ether:ethanol (1:1, 1 ml) and 4 methylphenyl boronic acid. The product was purified by flash column chromatography (iso-hexane:ethyl acetate, 5-66% gradient) to yield the title product as an oil (0.051 g, 38%). MS M+H 509. Retention Time 5.8 mins 30-90 MeCN:0.05% TFA 6 min Gradient C12 Reverse Phase 50 mm*4.6 mm i.d. column.

### Example 33

### (3aS,6S,6aS)-6-Fluoro-4-{(S)-4-methyl-2-[(S)-2,2,2-trifluoro-1-(4′-methyl-biphenyl-4-yl)-ethylamino]-pentanoyl}-tetrahydro-furo[3,2-b]pyrrol-3-one

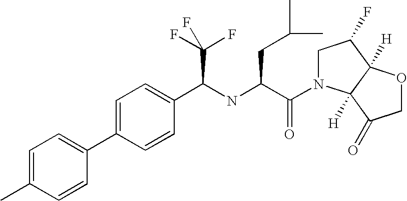

The technique described in Example 14 was applied to (S)-1-((3R,3aR,6S,6aS)-6-fluoro-3-hydroxy-hexahydro-furo[3,2-b]pyrrol-4-yl)-4-methyl-2-[(S)-2,2,2-trifluoro-1-(4′-methyl-biphenyl-4-yl)-ethylamino]-pentan-1-one (0.05 g, 0.10 mmol). The product was purified by prep-HPLC [Phenomenex Synergi C₁₂ 10 μm 10×150 mm column, 30→90% 15 min gradient of solution B (solution A=0.1% TFA in water and solution B=10% A in acetonitrile) at flow rate of 18 ml/min] to obtain the title compound, white solid (0.033 g, 66%) as a mixture with its ketone hydrate. MS M+H 507, M+H₂O+H 525. Retention Time 5.8 & 6.4 mins 30-90 MeCN:0.05% TFA, 6 min Gradient C12 Reverse Phase 50 mm*4.6 mm i.d. column.

### Example 34

### [(S)-1-(3-Bromo-phenyl)-2,2,2-trifluoro-ethyl]-r(S)-1-(tert-butyl-dimethyl-silanyloxymethyl)-3-methyl-butyl]-amine

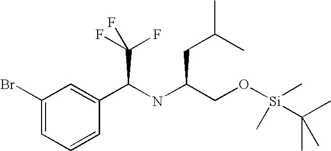

To a stirred solution of 1,3 dibromobenzene (9.1 g, 38.53 mmol) in dry THF (100 mls) under N2 at −78° C. was added dropwise 1.6M butyl lithium in hexanes (24.1 mls, 38.53 mmol). The resulting solution was stirred for a further 20 minutes at −78° C. after which a solution of [(S)-1-(tert-butyl-dimethyl-silanyloxymethyl)-3-methyl-butyl]-[2,2,2-trifluoro-eth-(E)-ylidene]-amine (4.0 g, 12.84 mmol) in dry THF (10 mls) was added dropwise to the solution of the aryl lithium. Stirring was continued for a further 1 hour at −78° C. The reaction mixture was quenched with 50 mls of 2M Hydrochloric acid solution and the mixture allowed to warm to room temperature. The solution was basified with 100 mls of Sodium Hydroxide and the resulting solution was extracted with ethyl acetate (2×100 mls) and the combined organic fractions were dried (MgSO₄) and concentrated in vacuo. The product was purified by reverse phase C18 column chromatography (H₂O:MeCN, 50-100% Gradient) to yield the title product as a yellow oil (2.55 g, 42%) MS M+H 468, Retention Time 8.3 mins 50-97 MeCN:0.05% TFA 6 min Gradient C12 Reverse Phase 50 mm*4.6 mm i.d. column.

### Example 35

### (S)-2-[(S)-1-(3-Bromo-phenyl)-2,2,2-trifluoro-ethylamino]-4-methyl-pentan-1-ol

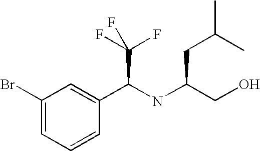

To a stirred solution of [(S)-1-(3-bromo-phenyl)-2,2,2-trifluoro-ethyl]-[(S)-1-(tert-butyl-dimethyl-silanyloxymethyl)-3-methyl-butyl]-amine (2.55 g, 5.4 mmol) in methanol (60 mls) was added concentrated hydrochloric acid (1 ml) and heated for 18 hours at 0° C. The reaction was allowed to cool and concentrated in vacuo. The product was purified by flash column chromatography (iso-hexane: ethyl acetate, 1-33% Gradient) to yield the title product as a yellow oil (1.57 g, 82%). MS M+H 354. Retention Time 4.1 mins 30-97 MeCN:0.05% TFA 6 min Gradient C12 Reverse Phase 50 mm*4.6 mm i.d. column.

### Example 36

### (S)-2-[(S)-1-(3-Bromo-phenyl)-2,2,2-trifluoro-ethylamino]-4-methyl-pentanoic acid

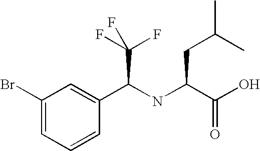

To a stirred suspension of periodic acid (11.5 g, 50.82 mmol) in acetonitrile (100 mls) was added chromium(VI) Oxide (23 mgs, 0.023 mmol) and water (0.25 mls). The resulting suspension was stirred for several hours at room temperature and then cooled to 0° C. on an ice bath and (S)-2-[(S)-1-(3-bromo-phenyl)-2,2,2-trifluoro-ethylamino]-4-methyl-pentan-1-ol (1.5 g, 4.19 mmol) was added dropwise in 10 mls of acetonitrile, stirring was continued at 0° C. for a further 30 minutes. The reaction was poured into acidified Na₂HPO₄ solution (12.0 g in 200 mls water adjusted to pH 3 with conc. HCl) and then extracted with diethyl ether (3×100 mls), the combined organic extracts were washed consecutively with brine (1×100 mls), sodium hydrogen sulphite solution (1×100 mls) and brine (1×100 mls). The organic extract was dried (Na₂SO₄) and concentrated to give the as title product as a yellow oil (1.3 g, 85%) MS M+H 368, Retention Time 5.0 mins 30-97 MeCN:0.05% TFA 6 min Gradient C12 Reverse Phase 50 mm*4.6 mm i.d. column.

### Example 37

### (S)-2-[(S)-1-(3-Bromo-phenyl)-2,2,2-trifluoro-ethylamino]-1-((3R,3aR,6S,6aS)-6-fluoro-3-hydroxy-hexahydro-furo[3,2-b]pyrrol-4-yl)-4-methyl-pentan-1-one

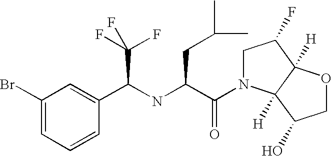

To a stirred solution of (3R,3aR,6S,6aS)-6-fluoro-3-hydroxy-hexahydro-furo[3,2-b]pyrrol-4-ium; chloride (0.71 g, 3.88 mmol) in DMF (10 mls) and (S)-2-[(S)-1-(3-bromo-phenyl)-2,2,2-trifluoro-ethylamino]-4-methyl-pentanoic acid (1.3 g, 3.53 mmol) was added HATU (1.6 g, 4.24 mmol) and diisopropyl ethyl amine (1.8 mls, 10.59 mmol). The resulting solution was stirred at room temperature overnight then concentrated in vacuo. The residue was then dispersed in water (50 mls) and extracted with ethyl acetate (2×150 mls). The combined organic fractions were washed with saturated sodium bicarbonate solution (1×100 mls), and dried over magnesium sulphate and concentrated. The product was purified by reverse phase C18 column chromatography (H₂O:Acetonitrile, 30-90% gradient) to yield the title product as a yellow oil (0.96 g, 55%) MS M+H 497, Retention Time 4.7 mins 30-90 MeCN:0.05% TFA 6 min Gradient C12 Reverse Phase 50 mm*4.6 mm i.d. column.

### Example 38

### (S)-1-((3R,3aR,6S,6aS)-6-Fluoro-3-hydroxy-hexahydro-furo[3,2-b]pyrrol-4-yl)-4-methyl-2-[(S)-2,2,2-trifluoro-1-(4′-methyl-biphenyl-3-yl)-ethylamino]-pentan-1-one

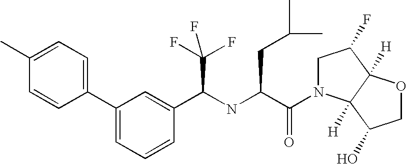

To a stirred solution of (S)-2-[(S)-1-(3-bromo-phenyl)-2,2,2-trifluoro-ethylamino]-1-((3R,3aR,6S,6aS)-6-fluoro-3-hydroxy-hexahydro-furo[3,2-b]pyrrol-4-yl)-4-methyl-pentan-1-one (0.15 g, 0.30 mmol) in dimethyl ether:ethanol (1:1, 1 ml) was added 4-methylsulfonyl phenyl boronic acid (0.044 g, 0.22 mmol), 2M sodium carbonate solution (1 ml) and polymer-supported tetrakis(triphenylphosphine)palladium(0) (0.15 g, 0.015 mmol). The resulting solution was sealed in a tube and heated in a microwave to 160° C. for 5 minutes. The reaction mixture was allowed to cool to room temperature and diluted with CH₂Cl₂:H₂O (1:1, 10 mls) and filtered. The organics were separated, dried (MgSO₄)) and concentrated in vacuo. The product was purified by flash column chromatography (iso-hexane:ethyl acetate, 5-66% gradient) to yield the title product as a clear oil (0.071 g, 48%). MS M+H 509. Retention Time 5.5 mins 10-90 MeCN:0.05% TFA 6 min Gradient C12 Reverse Phase 50 mm*4.6 mm i.d. column.

### Example 39

### (3aS,6S,6aS)-6-Fluoro-4-{(S)-4-methyl-2-[(S)-2,2,2-trifluoro-1-(4′-methyl-biphenyl-3-yl)-ethylamino]-pentanoyl}-tetrahydro-furo[3,2-b]pyrrol-3-one

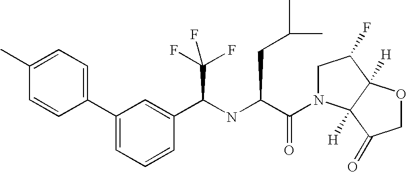

The technique described in Example 14 was applied to (S)-1-((3R,3aR,6S,6aS)-6-fluoro-3-hydroxy-hexahydro-furo[3,2-b]pyrrol-4-yl)-4-methyl-2-[(S)-2,2,2-trifluoro-1-(4′-methyl-biphenyl-3-yl)-ethylamino]-pentan-1-one (0.072 g, 0.14 mmol). The product was purified by prep-HPLC [Phenomenex Synergi C₁₂ 10 μm 10×150 mm column, 30→90% 15 min gradient of solution B (solution A=0.1% TFA in water and solution B=10% A in acetonitrile) at flow rate of 18 ml/min] to obtain the title compound, white solid (0.033 g, 46%) as a mixture with its ketone hydrate. MS M+H 507, M+H₂O+H 525. Retention Time 5.9 & 6.6 mins 30-90 MeCN:0.05% TFA, 6 min Gradient C12 Reverse Phase 50 mm*4.6 mm i.d. column.

### Example 40

### (S)-1-((3R,3aR,6S,6aS)-6-Fluoro-3-hydroxy-hexahydro-furo-[3,2-b]pyrrol-4-yl)-4-methyl-2-[(S)-2,2,2-trifluoro-1-(4′-methanesulfonyl-biphenyl-3-yl)-ethylamino]-pentan-1-one

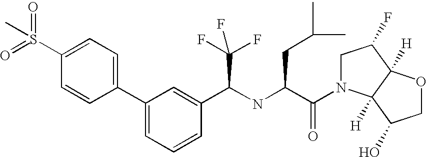

The technique described in Example 38 was applied to (S)-2-[(S)-1-(3-Bromo-phenyl)-2,2,2-trifluoro-ethylamino]-1-((3R,3aR,6S,6aS)-6-fluoro-3-hydroxy-hexahydro-furo[3,2-b]pyrrol-4-yl)-4-methyl-pentan-1-one (0.15 g, 0.30 mmol) and 4 methylsulfonylphenyl boronic acid. The product was purified by flash column chromatography (iso-hexane:ethyl acetate, 5-100% Gradient) to yield the title product as an oil (0.07 g, 41%). MS M+H 573. Retention Time 4.3 mins 30-90 MeCN:0.05% TFA 6 min Gradient C12 Reverse Phase 50 mm*4.6 mm i.d. column.

### Example 41

### (3aS,6S,6aS)-6-Fluoro-4-{(S)-4-methyl-2-[(S)-2,2,2-trifluoro-1-(4′-methanesulfonyl-biphenyl-3-yl)-ethylamino]-pentanoyl}-tetrahydro-furo[3,2-b]pyrrol-3-one

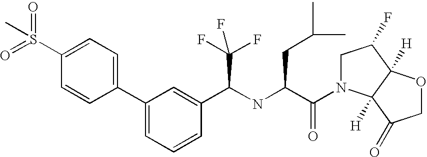

The technique described in Example 14 was applied to (S)-1-((3R,3aR,6S,6aS)-6-fluoro-3-hydroxy-hexahydro-furo[3,2-b]pyrrol-4-yl)-4-methyl-2-[(S)-2,2,2-trifluoro-1-(4′-methanesulfonyl-biphenyl-3-yl)-ethylamino]-pentan-1-one (0.07 g, 0.12 mmol). The product was purified by prep-HPLC [Phenomenex Synergi C₁₂ 10 μm 10×150 mm column, 30→90% 15 min gradient of solution B (solution A=0.1% TFA in water and solution B=10% A in acetonitrile) at flow rate of 18 ml/min] to obtain the title compound, white solid (0.027 g, 39%) as a mixture with its ketone hydrate. MS M+H 571, M+H₂O+H 589. Retention Time 5.2 & 5.4 mins 30-90 MeCN:0.05% TFA, 6 min Gradient C12 Reverse Phase 50 mm*4.6 mm i.d. column.

### Example 42

### (S)-1-((3R,3aR,6S,6aS)-6-Fluoro-3-hydroxy-hexahydro-furo[3,2-b]pyrrol-4-yl)-4-methyl-2-[(S)-2,2,2-trifluoro-1-(3′-methanesulfonyl-biphenyl-3-yl)-ethylamino]-pentan-1-one

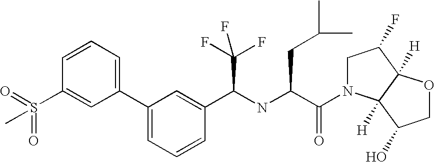

The technique described in Example 38 was applied to (S)-2-[(S)-1-(3-bromo-phenyl)-2,2,2-trifluoro-ethylamino]-1-((3R,3aR,6S,6aS)-6-fluoro-3-hydroxy-hexahydro-furo[3,2-b]pyrrol-4-yl)-4-methyl-pentan-1-one (0.15 g, 0.30 mmol) and 3 methylsulfonyl phenyl boronic acid. The product was purified by flash column chromatography (iso-hexane:ethyl acetate, 5-100% gradient) to yield the title product as an oil (0.1 g, 60%). MS M+H 573. Retention Time 4.4 mins 30-90 MeCN:0.05% TFA 6 min Gradient C12 Reverse Phase 50 mm*4.6 mm i.d. column.

### Example 43

### (3aS,6S,6aS)-6-Fluoro-4-{(S)-4-methyl-2-[(S)-2,2,2-trifluoro-1-(3′-methanesulfonyl-biphenyl-3-yl)-ethylamino]-pentanoyl}-tetrahydro-furo[3,2-b]pyrrol-3-one

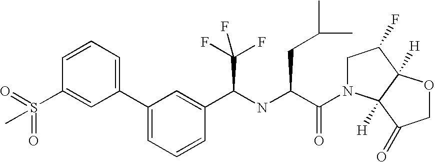

The technique described in Example 14 was applied to (S)-1-((3R,3aR,6S,6aS)-6-fluoro-3-hydroxy-hexahydro-furo[3,2-b]pyrrol-4-yl)-4-methyl-2-[(S)-2,2,2-trifluoro-1-(3′-methanesulfonyl-biphenyl-3-yl)-ethylamino]-pentan-1-one (0.1 g, 0.18 mmol). The product was purified by prep-HPLC [Phenomenex Synergi C₁₂ 10 μm 10×150 mm column, 30→90% 15 min gradient of solution B (solution A=0.1% TFA in water and solution B=10% A in acetonitrile) at flow rate of 18 ml/min] to obtain the title compound, white solid (0.051 g, 51%) as a mixture with its ketone hydrate. MS M+H 571, M+H₂O+H 589. Retention Time 5.3 & 5.5 mins 30-90 MeCN:0.05% TFA, 6 min Gradient C12 Reverse Phase 50 mm*4.6 mm i.d. column.

### Example 44

### (S)-1-((3R,3aR,6S,6aS)-6-Fluoro-3-hydroxy-hexahydro-furo[3,2-b]pyrrol-4-yl)-4-methyl-2-[(S)-2,2,2-trifluoro-1-(2′-fluoro-biphenyl-3-yl)-ethylamino]-pentan-1-one

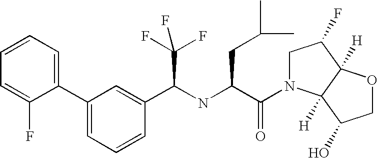

The technique described in Example 38 was applied to (S)-2-[(S)-1-(3-bromo-phenyl)-2,2,2-trifluoro-ethylamino]-1-((3R,3aR,6S,6aS)-6-fluoro-3-hydroxy-hexahydro-furo[3,2-b]pyrrol-4-yl)-4-methyl-pentan-1-one (0.15 g, 0.30 mmol) and 2-fluorophenyl boronic acid. The product was purified by flash column chromatography (iso-hexane:ethyl acetate, 5-66% gradient) to yield the title product as an oil (0.11 g, 69%). MS M+H 513. Retention Time 5.1 mins 30-90 MeCN:0.05% TFA 6 min Gradient C12 Reverse Phase 50 mm*4.6 mm i.d. column.

### Example 45

### (3aS,6S,6aS)-6-Fluoro-4-[(S)-4-methyl-2-{(S)-2,2,2-trifluoro-1-(2′-fluoro-biphenyl-3-yl)-ethylamino]-pentanoyl}-tetrahydro-furo[3,2-b]pyrrol-3-one

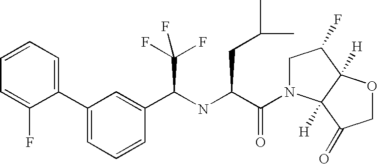

The technique described in Example 14 was applied to (S)-1-((3R,3aR,6S,6aS)-6-fluoro-3-hydroxy-hexahydro-furo[3,2-b]pyrrol-4-yl)-4-methyl-2-[(S)-2,2,2-trifluoro-1-(2′-fluoro-biphenyl-3-yl)-ethylamino]-pentan-1-one (0.11 g, 0.21 mmol). The product was purified by prep-HPLC [Phenomenex Synergi C₁₂ 10 μm 10×150 mm column, 30→90% 15 min gradient of solution B (solution A=0.1% TFA in water and solution B=10% A in acetonitrile) at flow rate of 18 ml/min] to obtain the title compound, white solid (0.054 g, 51%) as a mixture with its ketone hydrate. MS M+H 511, M+H₂O+H 529. Retention Time 5.4 & 6.1 mins 30-90 MeCN:0.05% TFA, 6 min Gradient C12 Reverse Phase 50 mm*4.6 mm i.d. column.

### Example 46

### (S)-1-((3R,3aR,6S,6aS)-6-Fluoro-3-hydroxy-hexahydro-furo[3,2-b]pyrrol-4-yl)-4-methyl-2-[(S)-2,2,2-trifluoro-1-(3-pyridin-4-yl-phenyl)-ethylamino]-pentan-1-one

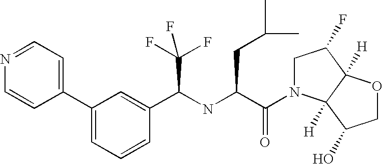

The technique described in Example 38 was applied to (S)-2-[(S)-1-(3-bromo-phenyl)-2,2,2-trifluoro-ethylamino]-1-((3R,3aR,6S,6aS)-6-fluoro-3-hydroxy-hexahydro-furo[3,2-b]pyrrol-4-yl)-4-methyl-pentan-1-one (0.15 g, 0.30 mmol) and 4-pyridyl boronic acid. The product was purified by flash column chromatography (iso-hexane:ethyl acetate, 5-100% gradient) to yield the title product as an oil (0.071 g, 47%). MS M+H 496. Retention Time 3.7 mins 30-90 MeCN:10 mM (NH₃)₂CO₃ 6 min Gradient C12 Reverse Phase 50 mm*4.6 mm i.d. column.

### Example 47

### 3aS,6S,6aS)-6-Fluoro-4-[(S)-4-methyl-2-{(S)-2,2,2-trifluoro-1-(3-pyridin-4-yl-phenyl)-ethylamino]-pentanoyl}-tetrahydro-furo[3,2-b]pyrrol-3-one

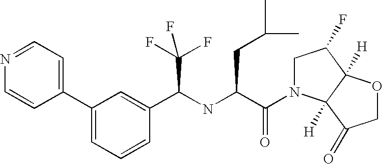

The technique described in Example 14 was applied to (S)-1-((3R,3aR,6S,6aS)-6-fluoro-3-hydroxy-hexahydro-furo[3,2-b]pyrrol-4-yl)-4-methyl-2-[(S)-2,2,2-trifluoro-1-(3-pyridin-4-yl-phenyl)-ethylamino]-pentan-1-one (0.071 g, 0.14 mmol). The product was purified by prep-HPLC [Phenomenex Synergi C₁₂ 10 μm 10×150 mm column, 30→90% 15 min gradient of solution B (solution A=0.1% TFA in water and solution B=10% A in acetonitrile) at flow rate of 18 ml/min] to obtain the title compound, white solid (0.008 g, 11%) as a mixture with its ketone hydrate. MS M+H 494, M+H₂O+H 512. Retention Time 4.0 & 4.6 mins 30-90 MeCN: 10 mM (NH₃)₂CO₃, 6 min Gradient C12 Reverse Phase 50 mm*4.6 mm i.d. column.

### Example 48

### 1-(4-Bromo-thiazol-2-yl)-4-methyl-piperazine

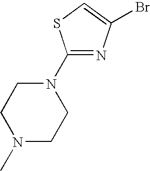

The title compound was prepared as shown in Palmer et al J. Med. Chem. 2005, 48, 7520-7534.

### Example 49

### (3R,3aR,6S,6aS)-6-Fluoro-3-hydroxy-hexahydro-furo[3,2-b]pyrrole-4-carboxylic acid benzyl ester

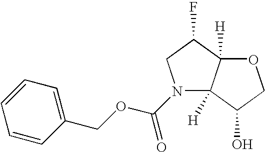

The title compound is prepared by conventional deprotection/protection from the building block of Example 1.

### Example 50

### (S)-2-[(S)-1-(4-Bromo-phenyl)-2,2,2-trifluoro-ethylamino]-4-methyl-pentanoic acid isopropyl ester

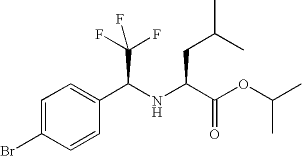

To a stirred solution of (S)-2-[(S)-1-(4-bromo-phenyl)-2,2,2-trifluoro-ethylamino]-4-methyl-pentanoic acid (1.80 g, 4.9 mmol) in isopropyl alcohol (100 mls) was added concentrated sulphuric acid (2 mls). The resulting solution was heated at 80° C. for 4 hours. The reaction mixture was allowed to cool concentrated in vacuo and the resulting oil dispersed in CH₂Cl₂ (100 mls) washed with saturated NaHCO₃ (2×50 mls), dried (MgSO₄) and concentrated in vacuo to yield the title compound as a brown oil (1.77 g, 88%). MS M+H 412. Retention Time 6.6 mins 30-97 MeCN:0.05% TFA 6 min Gradient C12 Reverse Phase 50 mm*4.6 mm i.d. column.

### Example 51

### (S)-4-Methyl-2-{(S)-2,2,2-trifluoro-1-[4-(4,4,5,5-tetramethyl-[1,3,2] dioxaborolan-2-yl)-phenyl]-ethylamino}-pentanoic acid isopropyl ester

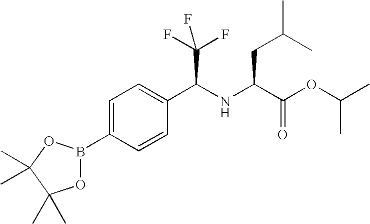

To a stirred solution of (S)-2-[(S)-1-(4-bromo-phenyl)-2,2,2-trifluoro-ethylamino]-4-methyl-pentanoic acid isopropyl ester (2.2 g, 5.36 mmol) in DMF (30 ml) was added bis(pinacolato) borane (2.0 g, 8.04 mmol), potassium acetate (1.6 g 16.1 mmol) and [1,1′-bis(diphenylphosphino)ferrocene]palladium(II) chloride 1:1 complex with CH₂Cl₂ (0.438 g, 0.54 mmol). The resulting solution was sealed in a tube and heated in a microwave to 160° C. for 20 minutes. The reaction mixture was allowed to cool to room temperature and filtered through a short silica column with ethyl acetate (500 mls). The resulting solution was concentrated in vacuo and the crude product was purified by reverse phase C18 column chromatography (H₂O: MeCN, 50-100% Gradient) to yield the title compound as a brown oil (0.920 g, 38%). MS M+H 458. Retention Time 4.6 mins 70-97 MeCN:0.05% TFA 6 min Gradient C12 Reverse Phase 50 mm*4.6 mm i.d. column.

### Example 52

### (S)-4-Methyl-2-((S)-2,2,2-trifluoro-1-{4-[2-(4-methyl-piperazin-1-yl)-thiazol-4-yl]-phenyl}-ethylamino)-pentanoic acid isopropyl ester

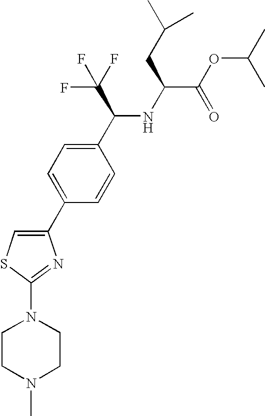

To a stirred solution of (S)-4-methyl-2-{(S)-2,2,2-trifluoro-1-[4-(4,4,5,5-tetramethyl-[1,3,2]dioxaborolan-2-yl)-phenyl]-ethylamino}-pentanoic acid isopropyl ester (0.72 g, 1.57 mmol) in DMF:H₂O (1:1, 20 mls) was added 1-(4-bromo-thiazol-2-yl)-4-methyl-piperazine (0.5 g, 1.89 mmol), sodium carbonate (0.2 g 1.89 mmol) and [1,1′-bis(diphenylphosphino)ferrocene]palladium(II) chloride 1:1 complex with CH₂Cl₂ (0.129 g, 0.16 mmol). The resulting solution was sealed in a tube and heated in a microwave to 160° C. for 20 minutes. The reaction mixture was allowed to cool and diluted with CH₂Cl₂ (100 mls). The organic phase was separated dried (MgSO₄) and concentrated in vacuo. The crude product was purified by flash column chromatography (ethyl acetate:MeOH, 9:1) to yield the title product as a dark red solid (0.150 g, 13%). MS M+H 513. Retention Time 4.0 mins 50-97 10 mM (NH₃)₂CO₃:MeCN 6 min Gradient C12 Reverse Phase 50 mm*4.6 mm i.d. column.

### Example 53

### (S)-4-Methyl-2-((S)-2,2,2-trifluoro-1-{4-[2-(4-methyl-piperazin-1-yl)-thiazol-4-yl]-phenyl}-ethylamino)-pentanoic acid; hydrogen chloride

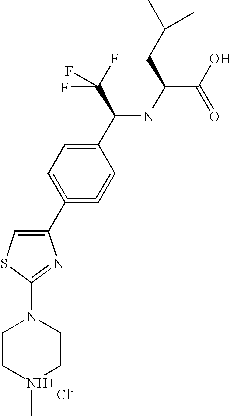

To a stirred mixture of 2M hydrochloric acid and dioxane (1:1, 10 mls) was added (S)-4-methyl-2-((S)-2,2,2-trifluoro-1-{4-[2-(4-methyl-piperazin-1-yl)-thiazol-4-yl]-phenyl}-ethylamino)-pentanoic acid isopropyl ester (0.15 g, 0.29 mmol). The solution was then heated for 20 hours at 100° C. and then concentrated in vacuo to dryness to give the title compound as a dark brown solid (0.14 g, 98%) which was used without any further purification. MS M−H 469.

### Example 54

### (3aS,6S,6aS)-6-Fluoro-3-oxo-hexahydro-furo[3,2-b]pyrrole-4-carboxylic acid benzyl ester

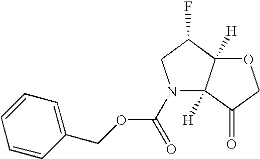

To a stirred solution of (3R,3aR,6S,6aS)-6-fluoro-3-hydroxy-hexahydro-furo[3,2-b]pyrrole-4-carboxylic acid benzyl ester (1.25 g, 4.44 mmol) in CH₂Cl₂ (50 mls) was added Dess-Martin periodinane (2.1 g, 4.44 mmol). The resulting solution was stirred at room temperature for 2 hours. The reaction mixture was quenched with a mixture of saturated NaHCO₃ and 10% Na₂S₂O₃ solution (1:1, 100 mls) an the organic phase dried(MgSO₄) and concentrated in vacuo. The crude product was purified by flash column chromatography (iso-hexane:ethyl acetate 1:1) to give the title compound as a clear oil (1.15 g, 92%). MS M+H 280, TLC R_{f} 0.2 (iso-hexane:ethyl acetate 1:2)

### Example 55

### (3aS,6S,6aS)-6-Fluoro-3,3-dimethoxy-hexahydro-furo[3,2-b]pyrrole-4-carboxylic acid benzyl ester

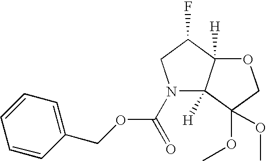

To a stirred solution of (3aS,6S,6aS)-6-fluoro-3-oxo-hexahydro-furo[3,2-b]pyrrole-4-carboxylic acid benzyl ester (1.15 g, 4.1 mmol) in methanol (10 mls) was added trimethyl orthoformate (5 mls) and p-toluene sulphonic acid) (0.02 g). The resulting solution was stirred at 60° C. for 3 hours and then allowed to cool and concentrated in vacuo. The crude product was purified by flash column chromatography (iso-hexane:ethyl acetate, 5-66% gradient) to give the title compound as a clear oil (1.07 g, 80%). MS M+H 326, TLC R_{f} 0.2 (iso-hexane:ethyl acetate 1:4)

### Example 56

### (3aS,6S,6aS)-6-Fluoro-3,3-dimethoxy-hexahydro-furo[3,2-b]pyrrole

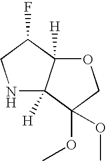

A solution of (3aS,6S,6aS)-6-fluoro-3,3-dimethoxy-hexahydro-furo[3,2-b]pyrrole-4-carboxylic acid benzyl ester (0.07 g, 0.22 mmol) in methanol (10 mls) was passed through a cartridge containing 10% Pd/C (10 mgs) on a H-Cube hydrogenator at a flow rate of 1 ml/min. The resulting solution was concentrated to give the title product as a clear oil (0.042 g, 99%). MS M+H 192.

### Example 57

### (S)-1-((3aS,6S,6aS)-6-Fluoro-3,3-dimethoxy-hexahydro-furo[3,2-b]pyrrol-4-yl)-4-methyl-2-((S)-2,2,2-trifluoro-1-{4-[2-(4-methyl-piperazin-1-yl)-thiazol-4-yl]-phenyl}-ethylamino)-pentan-1-one

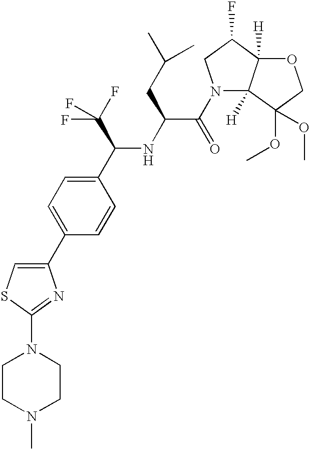

To a stirred solution of (3aS,6S,6aS)-6-Fluoro-3,3-dimethoxy-hexahydro-furo[3,2-b]pyrrole (0.084 g, 0.44 mmol) in DMF (10 mls) and (S)-4-methyl-2-((S)-2,2,2-trifluoro-1-{4-[2-(4-methyl-piperazin-1-yl)-thiazol-4-yl]-phenyl}-ethylamino)-pentanoic acid; hydrogen chloride (0.14 g, 0.29 mmol) was added HATU (0.17 g, 0.44 mmol) and diisopropyl ethyl amine (0.15 mls, 0.88 mmol). The resulting solution was stirred at room temperature overnight then concentrated in vacuo. The residue was then dispersed in CH₂Cl₂ (20 mls) and washed with saturated sodium bicarbonate solution (1×10 mls), and dried over magnesium sulphate and concentrated. The product was purified by reverse phase C18 column chromatography (H₂O:acetonitrile, 30-90% gradient) to yield the title compound as a light brown oil (0.39 g, 55%) MS M+H 644, Retention Time 4.7 mins 30-97 10 mM (NH₃)₂CO₃:MeCN 6 min Gradient C12 Reverse Phase 50 mm*4.6 mm i.d. column.

### Example 58

### (3aS,6S,6aS)-6-Fluoro-4-[(S)-4-methyl-2-((S)-2,2,2-trifluoro-1-{4-[2-(4-methyl-piperazin-1-yl)-thiazol-4-yl]-phenyl}-ethylamino)-pentanoyl]-tetrahydro-furo[3,2-b]pyrrol-3-one

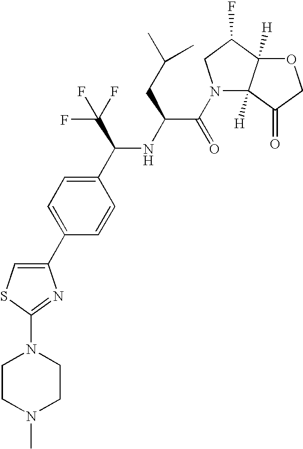

To a stirred solution of (S)-1-((3aS,6S,6aS)-6-fluoro-3,3-dimethoxy-hexahydro-furo[3,2-b]pyrrol-4-yl)-4-methyl-2-((S)-2,2,2-trifluoro-1-{4-[2-(4-methyl-piperazin-1-yl)-thiazol-4-yl]-phenyl}-ethylamino)-pentan-1-one (0.04 g, 0.062 mmol) in trifluoro acetic acid (1.8 mls) was added water (0.2 mls). The resulting solution was stirred for 24 hours at room temperature. The reaction mixture was diluted with CH₂Cl₂ (5 mls) and the solvent mixture evaporated using a flow of N₂ gas. The crude product was then redissolved in CH₂Cl₂ and neutralised with 2M Na₂CO₃ solution (1 ml), the organic layer was separated and concentrated in vacuo. The crude product was purified by prep-HPLC [Phenomenex Synergi C₁₂ 10 μm 10×150 mm column, 30→90% 15 min gradient of solution B (solution A=10 mM (NH₃)₂CO₃ in water and solution B=10% A in acetonitrile) to give the title product as a white solid (0.01 g, 27%) as a mixture with its ketone hydrate. MS M+H 598, M+H₂O+H 616. Retention Time 3.2 & 3.8 mins 30-97 10 mM (NH₃)₂CO₃:MeCN 6 min Gradient C12 Reverse Phase 50 mm*4.6 mm i.d. column.

### Example 59

### (S)-2-[1-(4-Bromo-phenyl)-2,2,2-trifluoro-ethylamino]-4-methyl-pentanoic acid

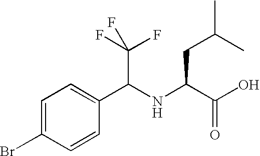

To a stirred suspension of periodic acid (11.6 g, 50.82 mmol) in acetonitrile (100 mls) was added chromium(VI) oxide (23 mgs, 0.023 mmol) and water (0.5 mls). The resulting suspension was stirred several hours at room temperature and then cooled to 0° C. on an ice bath and (S)-2-[1-(4-bromo-phenyl)-2,2,2-trifluoro-ethylamino]-4-methyl-pentan-1-ol (1.5 g, 4.23 mmol) was added dropwise in 10 mls of acetonitrile, stirring was continued at 0° C. for a further 30 minutes. The reaction was poured into acidified Na₂HPO₄ solution (12.0 g in 200 mls water adjusted to pH 3 with conc. HCl) and then extracted with diethyl ether (3×100 mls), the combined organic extracts were washed consecutively with brine (1×100 mls), sodium hydrogen sulphite solution (1×100 mls) and brine (1×100 mls). The organic extract was dried (Na₂SO₄) and concentrated to give the title compound as a yellow oil (1.61 g, 97%) which is a 2:1 mixture of diasterisomers. MS M+H 368. Retention Time 5.2 mins 10-90 MeCN:0.05% TFA 6 min Gradient C12 Reverse Phase 50 mm*4.6 mm i.d. column

### Example 60

### (S)-2-[1-(4-Bromo-phenyl)-2,2,2-trifluoro-ethylamino]-1-((3R,3aR,6S,6aS)-6-fluoro-3-hydroxy-hexahydro-furo[3,2-b]pyrrol-4-yl)-4-methyl-pentan-1-one

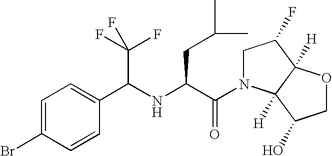

To a stirred solution of (3R,3aR,6S,6aS)-6-fluoro-3-hydroxy-hexahydro-furo[3,2-b]pyrrol-4-ium; chloride (0.09 g, 0.60 mmol) in CH₂Cl₂ (2 mls) and (S)-2-[1-(4-Bromo-phenyl)-2,2,2-trifluoro-ethylamino]-4-methyl-pentanoic acid (0.2 g, 0.54 mmol) was added DCC (0.22 g, 1.09 mmol) and diisopropyl ethyl amine (0.1 mls, 0.54 mmol). The resulting solution was stirred at room temperature overnight then filtered through celite and concentrated in vacuo. The product was purified by flash column chromatography (iso-hexane:Ethyl Acetate, 1:1) to yield the title compound as a yellow solid (0.155, 58%) MS M+H 497, Retention Time 5.8 mins 10-90 MeCN:0.05% TFA 6 min Gradient C12 Reverse Phase 50 mm*4.6 mm i.d. column.

### Example 61

### (3aS,6S,6aS)-6-Fluoro-4-{(S)-4-methyl-2-[2,2,2-trifluoro-1-(3′-methanesulfonyl-biphenyl-4-yl)-ethylamino]-pentanoyl}-tetrahydro-furo[3,2-b]pyrrol-3-one

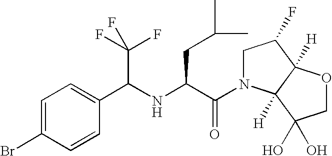

The technique described in Example 14 was applied to (S)-1-((3R,3aR,6S,6aS)-6-fluoro-3-hydroxy-hexahydro-furo[3,2-b]pyrrol-4-yl)-4-methyl-2-[2,2,2-trifluoro-1-(3′-methanesulfonyl-biphenyl-4-yl)-ethylamino]-pentan-1-one (0.120 g, 0.27 mmol). The product was purified by prep-HPLC [Phenomenex Synergi C₁₂ 10 μm 10×150 mm column, 30→90% 15 min gradient of solution B (solution A=0.1% TFA in water and solution B=10% A in acetonitrile) at flow rate of 18 ml/min] to obtain the title compound, white solid (0.036 g, 34%) as a mixture with its ketone hydrate. MS M+H 495, M+H₂O+H 513. Retention Time 5.9 & 6.4 mins 10-90 MeCN:0.05% TFA, 6 min Gradient C12 Reverse Phase 50 mm*4.6 mm i.d. column.

### Biological Examples

### Determination of Cathepsin K Proteolytic Catalytic Activity

Convenient assays for cathepsin K are carried out using human recombinant enzyme, such as that described in PDB.

ID BC016058 standard; mRNA; HUM; 1699 BP.

DE Homo sapiens cathepsin K (pycnodysostosis), mRNA (cDNA clone MGC:23107

RX MEDLINE; RX PUBMED; 12477932.

DR RZPD; IRALp962G1234.

DR SWISS-PROT; P43235;

The recombinant cathepsin K can be expressed in a variety of commercially available expression systems including E coli, Pichia and Baculovirus systems.

The purified enzyme is activated by removal of the prosequence by conventional methods.

Standard assay conditions for the determination of kinetic constants used a fluorogenic peptide substrate, typically H-D-Ala-Leu-Lys-AMC, and were determined in either 100 mM Mes/Tris, pH 7.0 containing 1 mM EDTA and 10 mM 2-mercaptoethanol or 100 mMNa phosphate, imM EDTA, 0.1% PEG4000 pH 6.5 or 100 mM Na acetate, pH 5.5 containing 5 mM EDTA and 20 mM cysteine, in each case optionally with 1M DTT as stabiliser. The enzyme concentration used was 5 nM. The stock substrate solution was prepared at 10 mM in DMSO. Screens were carried out at a fixed substrate concentration of 60 μM and detailed kinetic studies with doubling dilutions of substrate from 250 μM. The total DMSO concentration in the assay was kept below 3%. All assays were conducted at ambient temperature. Product fluorescence (excitation at 390 nm, emission at 460 nm) was monitored with a Labsystems Fluoroskan Ascent fluorescent plate reader. Product progress curves were generated over 15 minutes following generation of AMC product.

### Cathepsin S Ki Determination

The assay uses baculovirus-expressed human cathepsin S and the boc-Val-Leu-Lys-AMC fluorescent substrate available from Bachem in a 384 well plate format, in which 7 test compounds can be tested in parallel with a positive control comprising a known cathepsin S inhibitor comparator.

### Substrate Dilutions

280 μl/well of 12.5% DMSO are added to rows B-H of two columns of a 96 deep well polypropylene plate. 70 μl/well of substrate is added to row A. 2×250 μl/well of assay buffer (100 mM Na phosphate, 100 mM NaCl, pH 6.5) is added to row A, mixed, and double diluted down the plate to row H.

### Inhibitor Dilutions.

100 μl/well of assay buffer is added to columns 2-5 and 7-12 of 4 rows of a 96 well V bottom polypropylene plate. 200 μl/well of assay buffer is added to columns 1 and 6.

The first test compound prepared in DMSO is added to column 1 of the top row, typically at a volume to provide between 10 and 30 times the initially determined rough Kᵢ. The rough Ki is calculated from a preliminary run in which 10 μl/well of 1 mM boc-VLK-AMC (1/10 dilution of 10 mM stock in DMSO diluted into assay buffer) is dispensed to rows B to H and 20 μl/well to row A of a 96 well Microfluor™ plate. 2 μl of each 10 mM test compound is added to a separate well on row A, columns 1-10. Add 90 μl assay buffer containing 1 mM DTT and 2 nM cathepsin S to each well of rows B-H and 180 μl to row A. Mix row A using a multichannel pipette and double dilute to row G. Mix row H and read in the fluorescent spectrophotometer. The readings are Prism data fitted to the competitive inhibition equation, setting S=100 μM and K_{M}=100 μM to obtain an estimate of the Kᵢ, up to a maximum of 100 μM.

The second test compound is added to column 6 of the top row, the third to column 1 of the second row etc. Add 1 μl of comparator to column 6 of the bottom row. Mix column 1 and double dilute to column 5. Mix column 6 and double dilute to column 10.

Using an 8-channel multistepping pipette set to 5×10 μl, distribute 10 μl/well of substrate to the 384 well assay plate. Distribute the first column of the substrate dilution plate to all columns of the assay plate starting at row A. The tip spacing of the multichannel pipette will correctly skip alternate rows. Distribute the second column to all columns starting at row B.

Using a 12-channel multistepping pipette set to 4×10 μl, distribute 10 μl/well of inhibitor to the 384 well assay plate. Distribute the first row of the inhibitor dilution plate to alternate rows of the assay plate starting at A1. The tip spacing of the multichannel pipette will correctly skip alternate columns. Similarly, distribute the second, third and fourth rows to alternate rows and columns starting at A2, B1 and B2 respectively.

Mix 20 ml assay buffer and 20 μl 1M DTT. Add sufficient cathepsin S to give 2 nM final concentration.

Using the a distributor such as a Multidrop 384, add 30 μl/well to all wells of the assay plate and read in fluorescent spectrophotomoter such as an Ascent.

Fluorescent readings, (excitation and emission wavelengths 390 nm and 460 nm respectively, set using bandpass filters) reflecting the extent of enzyme cleavage of the fluorescent substrate, notwithstanding the inhibitor, are linear rate fitted for each well.

Fitted rates for all wells for each inhibitor are fitted to the competitive inhibition equation using SigmaPlot 2000 to determine V, Km and Ki values.

### Cathepsin L Ki

The procedure above with the following amendments is used for the determination of Ki for cathepsin L.

The enzyme is commercially available human cathepsin L (for example Calbiochem). The substrate is H-D-Val-Leu-Lys-AMC available from Bahcem. The assay buffer is 100 mM sodium acetate 1 mM EDTA, pH5.5) The DMSO stock (10 mM in 100% DMSO) is diluted to 10% in assay buffer. Enzyme is prepared at 5 nM concentration in assay buffer plus 1 mM dithiothreitol just before use. 2 ul of 10 mM inhibitor made up in 100% DMSO is dispensed into row A. 10 ul of 50 uM substrate (=1/200 dilution of 10 mM stock in DMSO, diluted in assay buffer)

### Inhibition Studies

Potential inhibitors are screened using the above assay with variable concentrations of test compound. Reactions were initiated by addition of enzyme to buffered solutions of substrate and inhibitor. Kᵢ values were calculated according to equation 1

$${v}_{0} = \frac{VS}{{K}_{M} ( 1 + \frac{I}{{K}_{i}} ) + S} \qquad ( 1 )$$

where v₀ is the velocity of the reaction, V is the maximal velocity, S is the concentration of substrate with Michaelis constant of K_{M}, and I is the concentration of inhibitor.

Reprepresentative compounds of the invention were assayed for cathepsin K potency and selectivity in the assays above. Note that the cathepsin L and S data is in micromolar, whereas the compounds are so potent against cathepsin K that the results are presented in nanomolar.

| | Cathepsin K IC₅₀ | | Cathepsin L IC₅₀ | | Cathepsin S IC₅₀ | |
|---|---|---|---|---|---|---|
| Example 14 | 29 | nM | 100 | uM | 36 | uM |
| Example 18 | 56 | nM | 94 | uM | 19 | uM |
| Example 20 | 180 | nM | 30 | uM | 37 | uM |
| Example 26 | 44 | nM | 25 | uM | 22 | uM |
| Example 28 | 51 | nM | 5.5 | uM | 10 | uM |
| Example 29 | 18 | nM | 180 | uM | 27 | uM |
| Example 31 | 70 | nM | 29 | uM | 19 | uM |
| Example 33 | 190 | nM | 20 | uM | 20 | uM |
| Example 58 | <5 | nM | 139 | uM | 188 | uM |
| Example 61 | 1 | nM | 6 | uM | 25 | uM |

It will be apparent that the compounds of the invention are very potent against cathepsin K but also at least 100 fold selectivity against the closely related cysteine proteases cathespin L and S. Additionally, the compounds typically possess good permeability (as measured below) and other DMPK properties.

### Permeability

This example measures transport of inhibitors through the cells of the human gastroenteric canal. The assay uses the well known Caco-2 cells with a passage number between 40 and 60.

### Apical to Basolateral Transport

Generally every compound will be tested in 2-4 wells. The basolateral and the apical wells will contain 1.5 mL and 0.4 mL transport buffer (TB), respectively, and the standard concentration of the tested substances is 10 μM. Furthermore all test solutions and buffers will contain 1% DMSO. Prior to the experiment the transport plates are pre-coated with culture medium containing 10% serum for 30 minutes to avoid nonspecific binding to plastic material. After 21 to 28 days in culture on filter supports the cells are ready for permeability experiments.

Transport plate no 1 comprises 3 rows of 4 wells each. Row 1 is denoted Wash, row 2 “30 minutes” and row 3 “60 minutes”. Transport plate no 2 comprises 3 rows of 4 wells, one denoted row 4 “90 minutes”, row 5 “120 minutes and the remaining row unassigned.

The culture medium from the apical wells is removed and the inserts are transferred to a wash row (No. 1) in a transport plate (plate no. 1) out of 2 plates without inserts, which have already been prepared with 1.5 mL transport buffer (HBSS, 25 mM HEPES, pH 7.4) in rows 1 to 5. In A→B screening the TB in basolateral well also contains 1% Bovine Serum Albumin.

0.5 mL transport buffer (HBSS, 25 mM MES, pH 6.5) is added to the inserts and the cell monolayers equilibrated in the transport buffer system for 30 minutes at 37° C. in a polymix shaker. After being equilibrated to the buffer system the Transepithelial electrical resistance value (TEER) is measured in each well by an EVOM chop stick instrument. The TEER values are usually between 400 to 1000Ω per well (depends on passage number used).

The transport buffer (TB, pH 6.5) is removed from the apical side and the insert is transferred to the 30 minutes row (No. 2) and fresh 425 μL TB (pH 6.5), including the test substance is added to the apical (donor) well. The plates are incubated in a polymix shaker at 37° C. with a low shaking velocity of approximately 150 to 300 rpm.

After 30 minutes incubation in row 2 the inserts will be moved to new pre-warmed basolateral (receiver) wells every 30 minutes; row 3 (60 minutes), 4 (90 minutes) and 5 (120 minutes).

25 μL samples will be taken from the apical solution after ˜2 minutes and at the end of the experiment. These samples represent donor samples from the start and the end of the experiment.

300 μL will be taken from the basolateral (receiver) wells at each scheduled time point and the post value of TEER is measured at the end the experiment. To all collected samples acetonitrile will be added to a final concentration of 50% in the samples. The collected samples will be stored at −20° C. until analysis by HPLC or LC-MS.

### Basolateral to Apical Transport

Generally every compound will be tested in 2-4 wells. The basolateral and the apical wells will contain 1.55 mL and 0.4 mL TB, respectively, and the standard concentration of the tested substances is 10 μM. Furthermore all test solutions and buffers will contain 1% DMSO. Prior to the experiment the transport plates are precoated with culture medium containing 10% serum for 30 minutes to avoid nonspecific binding to plastic material.

After 21 to 28 days in culture on filter supports the cells are ready for permeability experiments. The culture medium from the apical wells are removed and the inserts are transferred to a wash row (No. 1) in a new plate without inserts (Transport plate).

The transport plate comprises 3 rows of 4 wells. Row 1 is denoted “wash” and row 3 is the “experimental row”. The transport plate has previously been prepared with 1.5 mL TB (pH 7.4) in wash row No. 1 and with 1.55 mL TB (pH 7.4), including the test substance, in experimental row No. 3 (donor side).

0.5 mL transport buffer (HBSS, 25 mM MES, pH 6.5) is added to the inserts in row No. 1 and the cell monolayers are equilibrated in the transport buffer system for 30 minutes, 37° C. in a polymix shaker. After being equilibrated to the buffer system the TEER value is measured in each well by an EVOM chop stick instrument.

The transport buffer (TB, pH 6.5) is removed from the apical side and the insert is transferred to row 3 and 400 μL fresh TB, pH 6.5 is added to the inserts. After 30 minutes 250 μL is withdrawn from the apical (receiver) well and replaced by fresh transport buffer. Thereafter 250 μL samples will be withdrawn and replaced by fresh transport buffer every 30 minutes until the end of the experiment at 120 minutes, and finally a post value of TEER is measured at the end of the experiment. A 25 μL samples will be taken from the basolateral (donor) compartment after ˜2 minutes and at the end of the experiment. These samples represent donor samples from the start and the end of the experiment.

To all collected samples acetonitrile will be added to a final concentration of 50% in the samples. The collected samples will be stored at −20° C. until analysis by HPLC or LC-MS.

### Calculation

Determination of the cumulative fraction absorbed, FA_{cum}, versus time. FA_{cum} is calculated from:

$$F {A}_{cum} = \sum \frac{{C}_{RI}}{{C}_{DI}}$$

Where C_{Ri} is the receiver concentration at the end of the interval i and C_{Di} is the donor concentration at the beginning of interval i. A linear relationship should be obtained.

The determination of permeability coefficients (Pₐₚₚ, cm/s) are calculated from:

$${P}_{app} = \frac{( k \cdot {V}_{R} )}{( A \cdot 60 )}$$

where k is the transport rate (min⁻¹) defined as the slope obtained by linear regression of cumulative fraction absorbed (FA_{cum}) as a function of time (min), V_{R} is the volume in the receiver chamber (mL), and A is the area of the filter (cm²).

### Reference Compounds

| Category of absorption | | % absorption |
|---|---|---|
| in man | Markers | in man |
| PASSIVE TRANSPORT | | |
| Low (0-20%) | Mannitol | 16 |
| | Methotrexate | 20 |
| Moderate (21-75%) | Acyclovir | 30 |
| High (76-100%) | Propranolol | 90 |
| | Caffeine | 100 |
| ACTIVE TRANSPORT | | |
| Amino acid transporter | L-Phenylalanine | 100 |
| ACTIVE EFFLUX | | |
| PGP-MDR1 | Digoxin | 30 |

All references referred to in this application, including patents and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

1. A compound of the formula II 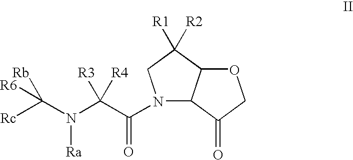
wherein
one of R¹ and R² is halo and the other is H or halo;
R³ is —C₁-C₅ straight or branched chain, optionally fluorinated, alkyl or —CH₂CR⁵C₃-C₄-cycloalkyl;
R⁴ is H;
R⁵ is H, C₁-C₂ alkyl, C₁-C₂ haloalkyl, hydroxyl, OC₁-C₂alkyl, fluoro;
R⁶ is a stable, optionally substituted, monocyclic or bicyclic, carbocycle or hetorocycle wherein the or each ring has 4, 5 or 6 ring atoms and 0 to 3 hetero atoms selected from S, O and N and wherein the optional substituents comprise 1 to 3 members selected from R₇;
R₇ is independently selected from halo, oxo, nitrite, nitro, C₁-C₄ alkyl, —XNRdRe, —XNReR⁸, —NReXR⁸, NH₂CO—, X—R⁸, X—O—R⁸, O—X—R⁸, X—C(═O)R⁸, X(C═O)NRdR⁸, X—NReC(═O)R⁸, X—NHSOmR⁸, X—S(═O)ₙ, R⁸, X—C(═O)OR⁸, XNReC(═O)OR⁸;
R⁸ is independently H, C₁-C₄ alkyl, C₃-C₆ cycloalkyl, pyrrolidinyl, piperidinyl, morpholinyl, thiomorpholinyl, piperazinyl, indolinyl, pyranyl, thiopyranyl, furanyl, thienyl, pyrrolyl, oxazolyl, isoxazolyl, thiazolyl, imidazolyl, pyridinyl, pyrimidinyl, pyrazinyl, indolyl, phenyl, any of which is optionally substituted with up to 3 members selected from R⁹;
R⁹ is independently selected from hydroxy, XR¹⁰, —XRdRe, —XNReR¹⁰, —NReC₁-C₄alkylR¹⁰, —S(═O)ₘRe, cyano, carboxy, oxo, C₁-C₄ alkyl, C₁-C₄haloalkyl, C₁-C₄-alkoxy, C₁-C₄ alkanoyl, carbamoyl;
R¹⁰ is C₃-C₆ cycloalkyl, pyrrolidinyl, piperidinyl, morpholinyl, thiomorpholinyl, piperazinyl, indolinyl, pyranyl, thiopyranyl, furanyl, thienyl, pyrrolyl, oxazolyl, isoxazolyl, thiazolyl, imidazolyl, pyridinyl, pyrimidinyl, pyrazinyl, indolyl, phenyl, any of which is optionally substituted with C₁-C₄ alkyl, halo, hydroxy, C₁-C₄alkoxy, cyano, —S(═O)ₘRe, C₁-C₄-haloalkyl;
X is independently a bond or C₁-C₄ alkylene;
Ra is independently H, C₁-C₄ alkyl or CH₃C(═O);
Rb is C₁-C₄ haloalkyl;
Rc is H, C₁-C₄ alkyl; or Re together with R⁶ and the carbon atom to which they are both attached form a carbocycle or heterocycle as defined for R⁶;
Rd is independently H, C₁-C₄ alkyl or CH₃C(═O);
Re is independently H, C₁-C₄ alkyl; or
Rd and Re together with the N atom to which they are attached form a morpholine, piperidine, piperazine or pyrrolidine ring optionally substituted with R⁹;
m is independently 0, 1 or 2;
or a pharmaceutically acceptable salt, hydrate or N-oxide thereof.

2. The compound according to claim 1, wherein the stereochemistry is as depicted in the partial structure below:

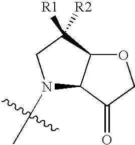

3. The compound according to claim 1, wherein the stereochemistry is as depicted in the partial structure below:

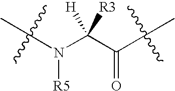

4. The compound according to claim 1, wherein Rb is trifluoromethyl and the stereochemistry is as depicted in the partial structure below:

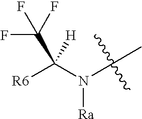

5. The compound according to claim 1, wherein R² is fluoro and R¹ is H.

6. The compound according to claim 1, wherein R³ is C₁-C₄ branched chain alkyl.

7. The compound according to claim 6, wherein R³ is iso-butyl.

8. The compound according to claim 1, wherein the Ra depicted in formula II is H.

9. The compound according to claim 1, wherein R⁶ is substituted phenyl.

10. The compound according to claim 9, wherein the substituent comprises —NRdRe, —CH₂NRdRe, —NReR⁹, —NReXR⁹, C₁-C₄ straight or branched alkyl or —O—R⁹.

11. The compound according to claim 10, wherein the substituent comprises —NH—CH₂ phenyl, —NHCH₂pyridyl or —NH-phenyl, wherein each phenyl or pyridyl ring is substituted with C₁-C₄-alkyl, —NRaRb, —NRbR⁸ or —NRbC₁-C₄alkylR⁸.

12. The compound according to claim 9, wherein the substituent comprises C₃-C₆ cycloalkyl, pyrrolidinyl, piperidinyl, morpholinyl, thiomorpholinyl, piperazinyl, indolinyl, pyranyl, thiopyranyl, furanyl, thienyl, pyrrolyl, oxazolyl, isoxazolyl, thiazolyl, imidazolyl, pyridinyl, pyrimidinyl, pyrazinyl, indolyl, phenyl, any of which is optionally substituted with R⁹.

13. The compound according to claim 12, wherein the substituent is selected from indolinyl, pyranyl, thiopyranyl, pyrrolyl, oxazolyl, isoxazolyl, thiazolyl, imidazolyl, pyridinyl, pyrimidinyl, pyrazinyl, indolyl, any of which is optionally substituted with R⁹.

14. The compound according to claim 13, wherein the substituent is thiazolyl, 5-methyl-thiazolyl or thienyl, any of which is optionally substituted with R⁹.

15. The compound according to claim 14, wherein the substituent is thiazol-4-yl, 5-methylthiazol-4-yl or thien-2-yl, any of which is optionally substituted with R⁹

16. The compound according to claim 15, wherein the thiazolyl, 5-methylthiazolyl or theinyl is substituted with morpholinyl, morpholinylmethyl-, piperidinyl, piperidinylmethyl-, piperazinyl, piperazinylmethyl, any of which is substituted with C₁-C₃ alkyl, fluoro, difluoro or C₁-C₃ alkyl-O—C₁-C₃alkyl.

17. The compound according to claim 16, wherein the substituent to the thiazolyl, 5-methylthiazolyl or thienyl is piperid-4-yl which is substituted with methyl, piperazinyl which is N-substituted with C₁-C₃ alkyl or methyloxyethyl-, - or piperid-1-ylmethyl-which is unsubstituted or 4-substituted with fluoro or difluoro.

18. The compound according to claim 10, wherein the substituent comprises a morpholine, piperidine or piperazine ring, optionally substituted with R⁹.

19. The compound according to claim 18 comprising piperid-4-yl or N-piperazinyl, N-substituted with Ra or piperidin-1-yl which is 4-substituted with —NRdRe.

20. The compound according to claim 1, wherein R⁶ is benzothiazolyl, benzofuranyl, 3-methylbenzofuranyl or benzoxazolyl, any of which is optionally substituted with one or two R⁷.

21. The compound according to claim 20, wherein one such substituent is —OR⁸, —OXR⁸, —NReR⁸ or —NReXR⁸.

22. The compound according to claim 21, wherein R⁸ is piperid-4-yl, piperazin-1-yl or piperidin-1-yl or morpholino, any of which is substituted with C₁-C₃ alkyl.

23. The compound according to claim 22, wherein the optional substituent to R⁶ is N-morpholinylethyloxy, N-morpholinylmethyloxy, N-methylpiperid-4-yloxy, or N-methylmorpholin-3-ylmethyloxy.

24. The compound according to claim 1, wherein the optional substituent R⁹ is selected from hydroxy, XR¹⁰, —XNRdRe, —XNReR¹⁰, —NReC₁-C₄alkylR¹⁰, -cyano, carboxy, oxo, C₁-C₄ alkyl, C₁-C₄-alkoxy, C₁-C₄ alkanoyl or carbamoyl.

25. A pharmaceutical composition comprising a compound as defined in claim 1 and a pharmaceutically acceptable carrier or diluent therefor.

26. (canceled)

27. The method according to claim 30, wherein the disorder is selected from: osteoporosis,
gingival diseases such as gingivitis and periodontitis,
Paget's disease,
hypercalcaemia of malignancy,
metabolic bone disease,
diseases characterised by excessive cartilage or matrix degradation, such as
osteoarthritis and rheumatoid arthritis,
bone cancers including neoplasia pain.

28. (canceled)

29. (canceled)

30. A method for the treatment of a disorder characterised by inappropriate expression or activation of cathepsin K in mammals having or being identified as being at risk of developing said disorder comprising the administration of a safe and effective amount of a compound according to claim 1 to a subject in need thereof.
